(19) United States
(12) Patent Application Publication
(10) Pub. No.: US 2025/0302885 A1
(43) Pub. Date: Oct. 2, 2025

(54) RHODOSPIRILLUM RUBRUM CELLS THAT LOWER PLASMA CHOLESTEROL WHILE LEAVING OTHER SERUM PROTEIN LEVELS UNAFFECTED

(71) Applicant: Curador, Liège (BE)

(72) Inventors: Jogchum Plat, Geulle (NL); Ronald Peter Mensink, Echt (NL); Robertus Christiaan Josephus Suters, Rosmalen (NL)

(73) Assignee: Curador, Liège (BE)

(21) Appl. No.: 19/047,372

(22) Filed: Feb. 6, 2025

Related U.S. Application Data
(62) Division of application No. 17/619,594, filed on Dec. 16, 2021, now abandoned.

(30) Foreign Application Priority Data
Jun. 25, 2019 (NL) 2023371
Nov. 20, 2019 (NL) 2024271

(51) Int. Cl. A61K 35/74; A23L 33/135; A61K 35/00; A61P 3/06

(52) U.S. Cl. CPC A61K 35/74; A61P 3/06; A23L 33/135; A61K 2035/115

(57) ABSTRACT

The present invention relates to a composition comprising Rhodospirillum rubrum cells for use in a method for inhibiting the uptake of cholesterol from the intestine in a human subject, such that the plasma LDL-cholesterol level in said human subject is lowered and concentrations of blood proteins relating to kidney function, liver function and/or heart function are essentially unaltered. In addition, the invention relates to a non-therapeutic method of lowering plasma LDL-cholesterol level in a subject, the method comprising administering to the subject a composition comprising Rhodospirillum rubrum cells, wherein lowering of the plasma LDL-cholesterol level is the inhibition of the uptake of cholesterol from the intestine in said human subject. Furthermore, the invention relates to a food product, a feed product, a food stuff and a food supplement comprising Rhodospirillum rubrum cells and having plasma LDL-cholesterol level lowering properties relating to intestinal cholesterol uptake inhibitory properties.

### CROSS-REFERENCES TO RELATED APPLICATIONS

This application is a divisional of U.S. patent application Ser. No. 17/619,594, filed Dec. 16, 2021, which is a national stage of International Application No. PCT/NL2020/050389, filed Jun. 18, 2020, which claims priority to NL Application No. 2024271, filed Nov. 20, 2019, and NL Application No. 2023371, filed Jun. 25, 2019, the disclosures of which are incorporated by reference herein in their entirety.

### TECHNOLOGICAL FIELD

The present invention relates to a composition comprising Rhodospirillum rubrum (R. rubrum) cells for use in a method for inhibiting the uptake of cholesterol from the intestine in a human subject, such that the plasma LDL-cholesterol level in said human subject is lowered. The present invention also relates to a composition comprising Rhodospirillum rubrum cells for use in a method for lowering LDL-cholesterol in a human subject in need thereof. In addition, the invention relates to a non-therapeutic method of lowering plasma LDL-cholesterol level in a subject, the method comprising administering to the subject a composition comprising Rhodospirillum rubrum cells, wherein lowering of the plasma LDL-cholesterol level is the inhibition of the uptake of cholesterol from the intestine in said human subject. Furthermore, the invention relates to a food product, a feed product, a food stuff and a food supplement comprising Rhodospirillum rubrum cells and having plasma LDL-cholesterol level lowering properties relating to intestinal cholesterol uptake inhibitory properties. The invention also relates to the use of dried Rhodospirillum rubrum cells obtained with refractive drying for the manufacture of a medicament or a food supplement for the inhibition of uptake of cholesterol from the intestine of a human subject in need thereof, or for the lowering of the plasma level of LDL-cholesterol in a human patient in need thereof. Finally, the invention relates to a method for treating a human subject with Rhodospirillum rubrum cells, wherein the human subject optionally is suffering from a cardiovascular disease, the method comprising the steps of: determining the plasma level of LDL-cholesterol in said human subject by: 1) obtaining or having obtained a blood sample from the human subject; 2) performing or having performed a LDL-cholesterol concentration determining assay on the blood sample to determine if the patient has an LDL cholesterol concentration of at least 70 mg/dL (1.8 mmol/L); and 3) if the human subject has an LDL cholesterol concentration of at least 70 mg/dL (1.8 mmol/L) then internally administering Rhodospirillum rubrum cells to the patient.

### BACKGROUND ART

Cholesterol is essential for mammalian life, e.g. human life, as a structural component of cellular membranes, influencing membrane organization and thereby membrane properties. Cholesterol is also the precursor molecule of steroid hormones and therefore, essential for metabolic control. In the liver, cholesterol is converted into bile salts, which represents the major pathway for cholesterol metabolism in quantitative sense. Bile salts are amphipathic molecules that facilitate the absorption of dietary cholesterol, fats and fat-soluble vitamins in the small intestine. Cholesterol is a key component in cellular and whole-body physiology and cholesterol homeostasis is tightly regulated at a variety of levels.

Maintenance of cholesterol homeostasis in the body requires accurate metabolic cross-talk between processes that govern de novo cholesterol synthesis and turnover to adequately cope with (large) fluctuations in dietary cholesterol intake. Imbalance may lead to elevated plasma cholesterol levels such as high low-density lipoprotein-cholesterol levels, and increased risk for cardiovascular diseases (CVD), the main cause of death in Western society. There is a direct link between high plasma low density lipoprotein (LDL) cholesterol and risk for CVD. CVDs are responsible for over 17.3 million deaths per year and are the leading causes of death in the world, according to the World Health Organization. CVDs include diseases of the heart, vascular diseases of the brain and diseases of blood vessels. The different types of CVDs are: CVDs due to atherosclerosis, which are ischaemic heart disease or coronary artery disease (e.g. heart attack); cerebrovascular disease (e.g. stroke); diseases of the aorta and arteries, including hypertension and peripheral vascular disease, and other CVDs, i.e. congenital heart disease; rheumatic heart disease; cardiomyopathies; and cardiac arrhythmias.

CVD is caused by a number of synergistic factors, the most important being a too high blood cholesterol level. As said, cholesterol is an essential building block for animal and human cells, since it is a component of cell membranes. Human cells can synthesize their own cholesterol, but cholesterol is thus also assimilated from food. Both processes play an important part in cholesterol metabolism.

Apart from its essential biological role as a building block for cellular membranes, cholesterol indeed also has negative effects on human health, as a cause of cardiovascular disease (such as, for instance, myocardial infarction, stroke, and peripheral vascular disease), more specifically relating to the occurrence of atherosclerotic lesions in the blood vessel wall. An elevated plasma cholesterol level is the most important predictive risk factor for the occurrence of cardiovascular disease and atherosclerosis.

In blood plasma, cholesterol is transported in lipoproteins, which can be subdivided into a number of different classes, based on their diameter and specific density. The very-low-density lipoproteins (VLDL), the intermediate-density lipoproteins (IDL), the low-density lipoproteins (LDL), and the high-density lipoproteins (HDL) constitute the most important classes of lipoproteins.

Experimental studies and clinical studies have shown that the amount of cholesterol transported in the VLDL, IDL and LDL classes of lipoproteins (the pro-atherogenic cholesterol; ‘bad’ cholesterol) is a risk factor for the occurrence of cardiovascular disease. Cholesterol transported in HDL particles, in contrast, protects against the development of cardiovascular disease (anti-atherogenic cholesterol; ‘good’ cholesterol).

Randomized-controlled, placebo-controlled, prospective clinical studies have demonstrated that lowering plasma cholesterol has a favorable effect on the incidence of cardiovascular disease and on mortality, particularly when LDL-cholesterol plasma levels are reduced. A prerequisite is, though, therefore that the reduction in cholesterol should be predominantly or substantially due to a reduction in the pro-atherogenic cholesterol present in LDL, leaving the level of anti-atherogenic cholesterol (HDL-cholesterol) preferably essentially unaltered.

Research has additionally shown that a high-risk lipoprotein profile, i.e. high LDL-cholesterol plasma levels, is associated with a higher resting heart rate, which in turn is associated with a higher risk of cardiovascular disease. On the other hand, higher (relative) levels of HDL-cholesterol have a positive effect on resting heart rate.

For the treatment and prevention of cardiovascular disease it is therefore imperative to reduce the pro-atherogenic (bad) cholesterol such as the level of LDL-cholesterol, and to increase, in absolute or relative proportion, the anti-atherogenic (good) cholesterol, the HDL-cholesterol.

A number of approaches are available to reduce plasma cholesterol. The most important are:
to inhibit cholesterol biosynthesis;
to increase the removal of cholesterol (and/or its metabolites, specifically bile acids) from tissues into the intestinal lumen;
to reduce the absorption of cholesterol and bile acids from the gastrointestinal tract.

Drugs that are used to inhibit cholesterol synthesis are often inhibitors of the enzyme hydroxymethyl-glutaryl-coenzyme A reductase (HMGCoA reductase), the rate-limiting enzyme in the cholesterol synthesis pathway. These “statins” are molecules that inhibit enzyme action. These statins, or HMG-CoA reductase inhibitors, are a class of lipid-lowering medications that reduce illness and mortality in those who are at high risk of cardiovascular disease. Examples are simvastatin, pravastatin and atorvastatin. Statins are generally chemically-synthetized derivatives of naturally-occurring fungal metabolites. Treatment of high plasma cholesterol has been focused for many years on interference with cholesterol synthesis by application of such statins. However, a relative large number of hypercholesterolaemic patients do not adequately respond to statin therapy or remain at risk for CVD despite substantial reductions in LDL cholesterol. Side effects of statins include muscle pain, increased risk of diabetes mellitus, and abnormal blood levels of liver enzymes. Additionally, statins have rare but severe adverse effects, particularly muscle damage.

Consequently, alternative strategies are currently actively pursued, particularly high density lipoprotein (HDL)-raising approaches. These approaches are considered particularly promising, as data from epidemiological studies indicate that every 1 mg/dL increase in HDL cholesterol reduces CVD risk by 2%-3%.

Extended release niacin has been reported to lower LDL-cholesterol with 17%. Fenofibrate has been reported to lower LDL-cholesterol levels with about 20%. Ezetimibe is an intestinal cholesterol absorption inhibitor which reduces LDL-cholesterol with 18%. Ezetimibe inhibits the absorption of cholesterol from the small intestine and decreases the amount of cholesterol normally available to liver cells, leading the liver cells to absorb more from circulation, thus lowering levels of circulating cholesterol. Ezetimibe blocks the critical mediator of cholesterol absorption, the Niemann-Pick C1-like 1 protein on the gastrointestinal tract epithelial cells, as well as in hepatocytes; it blocks aminopeptidase N and interrupts a caveolin 1-annexin A2 complex involved in trafficking cholesterol. Colesevelam is a bile acid sequestrant which reduces LDL-cholesterol with 18%. Mipomersen is an inhibitor of apolipoprotein B-100 synthesis and was shown to reduce LDL-cholesterol levels with about 25% in patients with homozygous familial hypercholesterolemia (reported adverse events: liver damage). Lomitapide is an inhibitor of microsomal triglyceride transfer protein for example for the treatment of patients with homozygous familial hypercholesterolemia (side-effects: fat accumulation in the liver). The lomitapide reduced LDL-cholesterol levels with 50% in those patients. Proprotein convertase subtilisin/kexin type 9 inhibitor (PCSK9 inhibitor) molecules and gene-silencing approaches are under development. Inhibition of PCSK9 in a subject may enhance the LDL-cholesterol lowering activity of statins. Combined treatment of subjects with an antibody against PCSK9 (REGN727/SAR236553) and statin atorvastatin resulted in a reduction in LDL-cholesterol levels of about 39% to 61%. The small molecule ETC-1002 modulates adenosine triphosphate-citrate lyase as well as adenosine monophosphate-activated protein kinase. In patients suffering from hypercholesterolemia (LDL-cholesterol levels of 130-220 mg/dL), LDL-cholesterol levels were reduced with about 18% to about 27%, when treated with increasing doses of ETC-1002. Cholesteryl ester transfer protein (CETP) inhibitors raise HDL-cholesterol levels and decrease LDL-cholesterol levels. Examples of such a CETP inhibitors are anacetrapib and evacetrapib. Anacetrapib and evacetrapib have been shown in clinical trials with human subjects to increase HDL-cholesterol levels with respectively about 138% and about 129%, and to lower LDL-cholesterol levels with respectively about 40% and about 36%. However, several of these CETP inhibitors, while lowering LDL-cholesterol levels, were unable to provide benefits when preventing cardiovascular events is considered. An example of a CETP inhibitor that has been discontinued during clinical trials in human, is evacetrapib, which failed to show a reduction in cardiovascular events. WAY-252623 is an activator of the beta-isoform of the liver X receptors. In non-human primates, WAY-252623 reduced LDL-cholesterol with 70%-77%.

It is known from several studies that some statins, including atorvastatin, lead to hepatoxicity, which is shown by a significant, dose-dependent, increase in serum levels of e.g. AST, ALT and γGT. Furthermore, nephrotoxicity has also been observed in relation to statin administration. Atorvastatin was observed to cause acute kidney injury (AKI). A further increase in NT-ProBNP levels is observed when using atorvastatin in a group of patients that already had a higher risk at cardiovascular events. This was also observed when atorvastatin was combined with ezetimibe. Statins have also been shown to increase resting heart rate in healthy human subjects; a higher resting heart rate is associated with a higher risk of CVD and is therefore normally undesirable. Fenofibrate increases creatinine levels in patients with normal kidney function. This indicates that continued fenofibrate treatment can lead to problems in kidney function.

Development of evacetrapib was discontinued due to an observation of increased hypertension in high-risk patients when compared to placebo. An increase was seen of hs-CRP, which was not observed in the placebo group, which relates to the observed increase in hypertension prevalence in the evacetrapib patient group.

The liver is considered the major control center of the body for maintenance of whole body cholesterol homeostasis. The liver is the main site for de novo cholesterol synthesis, clears cholesterol-containing chylomicron remnants and LDL particles from plasma and is the major contributor to high density lipoprotein (HDL; good cholesterol) formation. The liver has a central position in the classical definition of the reverse cholesterol transport pathway by taking up periphery-derived cholesterol from lipoprotein particles followed by conversion into bile acids or its direct secretion into bile for eventual removal via the feces. To increase cholesterol removal, a bile acid-adsorbing resin can be used (for example cholestyramine). Because of the adsorption of bile acids to the resin, their secretion in the stool is increased, and their reabsorption from the gut into the blood is reduced, resulting in a relative loss of bile acids from the body. Consequently, the liver increases the conversion of cholesterol into bile acids, resulting in a net increase in the secretion of cholesterol (metabolites) from the body. Because bile acids (by solubilizing cholesterol) are essential for the uptake of cholesterol from the lumen into the intestinal tissue, a reduction in bile acid content in the intestinal lumen will also result in a decreased cholesterol uptake.

As said, maintenance of cholesterol homeostasis is vital for healthy status and achieved through a regulatory network consisting of genes involved in cholesterol synthesis, absorption, metabolism and elimination. Imbalance of cholesterol level as a results of environmental and genetic factors leads to hypercholesterolemia, a predominant risk factor for atherosclerosis (i.e. hardening or furring of the arteries) and associated coronary and cerebrovascular diseases. Hypercholesterolemia and its associated cardiovascular diseases represent one of the greatest worldwide economic, social and medical challenges that we are facing now.

Despite the wide use of therapeutic drugs for controlling blood cholesterol, like statins inhibiting cholesterol synthesis, the fact remains that it is estimated that more than 50% of the population of the United States has cholesterol levels at the borderline levels. In addition, adverse effects associated with therapeutic drugs to control cholesterol levels, such as myopathy, liver damages and potential drug-drug interaction, have been reported. Therefore, development of additional therapies for controlling cholesterol levels is warranted, especially for those with better safety profiles.

Patent EP1569667 discloses a preparation of dead Rhodospirillum rubrum cells or freeze-dried Rhodospirillum rubrum cells, the freeze-dried cells tested for the lowering of plasma cholesterol in rats and mice. Similarly, international patent application WO2018/229223A1 discloses a petroleum-ether extract retrieved from freshly cultured Rhodospirillum rubrum (R. rubrum) cells and tested for the lowering of plasma cholesterol in mice. The last 20 years intensive research is conducted on the effects of consuming freeze-dried R. rubrum cells or R. rubrum extracts from freshly cultured cells on animal non-human cholesterol homeostasis.

### SUMMARY

There exists a need for safe ‘bad’ cholesterol-lowering methods and LDL-cholesterol-lowering compounds and compositions, specifically for safe use in the lowering of plasma LDL-cholesterol concentration in human subjects in need thereof, in particular efficacious methods and compositions or compounds having an acceptable and/or improved safety profile.

Despite the long period in which effects of R. rubrum cells on plasma cholesterol levels in non-human mammals has been studied, evidence-based data and insights on effects of R. rubrum cells on cholesterol homeostasis in (healthy) human subjects, obtained with randomized-controlled, double-blind clinical trials conducted according to the “gold standard”, are up to date not available to healthcare professionals and life-style intervention professionals, in order for providing an improved method for controlling and lowering LDL-cholesterol levels in blood of human subjects in need thereof, e.g. exerting less side effects, e.g. applicable for reducing risk for CVD in yet inadequately treated human subjects or even untreated human subjects, such as otherwise healthy human subjects with for example borderline levels of cholesterol, e.g. LDL-cholesterol.

It is therefore a goal of the current invention to provide LDL-cholesterol-lowering methods, compounds and compositions with an improved safety profile, specifically for safe and efficacious use in the lowering of plasma LDL-cholesterol concentration in human subjects in need thereof.

The present invention will be described with respect to particular embodiments but the invention is not limited thereto but only by the claims.

The embodiments of the invention described herein can operate in combination and cooperation, unless specified otherwise.

An aspect of the invention relates to Rhodospirillum rubrum cells for use in a method for lowering of LDL-cholesterol concentration in blood of a human subject.

An aspect of the invention relates to Rhodospirillum rubrum cells for use in the treatment or the prophylaxis of a cardiovascular disease in a human subject.

An aspect of the invention relates to Rhodospirillum rubrum cells for use in the treatment or prophylaxis of atherosclerosis, dyslipidemia, arteriosclerosis, hypercholesterolemia, familial hypercholesterolemia, hyperlipidemia, an LDL-cholesterol plasma level of at least 70 mg/dL, an LDL-cholesterol plasma level of at least 100 mg/dL, an LDL-cholesterol plasma level of at least 140 mg/dL, an LDL-cholesterol plasma level of at least 200 mg/dL, a total plasma cholesterol level of at least 200 mg/dL, a total plasma cholesterol level of 5.0 mM-8.0 mM, a plasma Lp(a) level of at least 14 mg/dL, ischemia, in a human subject.

An aspect of the invention relates to Rhodospirillum rubrum cells for use in the inhibition of absorption of cholesterol from the intestine of a human subject, such that LDL-cholesterol concentration in the blood of said human subject decreases or is decreased. The inventors established that administering the Rhodospirillum rubrum cells to human subjects is beneficial when the effect of lowering blood LDL-cholesterol is concerned, and moreover is safe and without side-effects. Human subjects to whom the cells were orally administered twice daily did not experience any side-effect or negative impact on their health or well-being relating to said daily intake. Moreover, assessing the blood concentrations of glucose, ALT, AST, γGT, creatinine, NT-ProBNP, vWF, c-Troponin T, and hsCRP, before, during and after twice daily oral intake of Rhodospirillum rubrum cells by human subjects revealed that these concentrations essentially remained unaltered and unaffected during and after the treatment, i.e. the administration of the cells. Further, these blood concentrations remained within the parameter range boundaries that are accepted as being standard normal and healthy values. Assessing the SBP and the DBP before, during and after twice daily oral intake of Rhodospirillum rubrum cells by human subjects also revealed that these blood pressures essentially remained unaltered and unaffected during and after the treatment, i.e. the administration of the cells. Further, these blood pressures remained within the parameter range boundaries that are accepted as being standard normal and healthy values.

An embodiment is the Rhodospirillum rubrum cells for use according to the invention, wherein the HDL-cholesterol concentration in the blood of the human subject remains unaltered or increases or is increased.

An aspect of the invention relates to a non-therapeutic method of lowering LDL-cholesterol concentration in blood of a human subject, the method comprising administering to the human subject a composition comprising of or consisting of Rhodospirillum rubrum cells.

An aspect of the invention relates to a non-therapeutic method of lowering LDL-cholesterol concentration in blood of a human subject, the method comprising orally administering to the human subject a composition comprising of or consisting of Rhodospirillum rubrum cells.

An embodiment is the Rhodospirillum rubrum cells for use according to the invention or the non-therapeutic method of the invention, wherein the concentration of at least one of ALT, AST, γGT, creatinine, NT-ProBNP, vWF, c-Troponin T, plasma glucose and hsCRP remains within a standard normal range and/or remains essentially constant (in said standard normal range) in the serum and/or plasma of the human subject upon administration of the Rhodospirillum rubrum cells to said human subject, preferably all the concentrations remain essentially constant and/or remain within the standard normal range, preferably compared to the concentration(s) determined for said at least one of ALT, AST, γGT, creatinine, NT-ProBNP, vWF, c-Troponin T, plasma glucose and hsCRP before the start of the first administration of the Rhodospirillum rubrum cells and/or during the subsequent further administration(s) of the Rhodospirillum rubrum cells.

An embodiment is the Rhodospirillum rubrum cells for use according to the invention or the non-therapeutic method of the invention, wherein diastolic blood pressure and/or systolic blood pressure of the human subject remain(s) within a standard normal range and/or remain(s) essentially constant upon administration of the Rhodospirillum rubrum cells to said human subject, preferably compared to said blood pressure(s) determined before the start of the first administration of the Rhodospirillum rubrum cells and/or during the subsequent further administration(s) of the Rhodospirillum rubrum cells.

An embodiment is the Rhodospirillum rubrum cells for use according to the invention or the non-therapeutic method of the invention, wherein the resting heart rate of the human subject remains within a standard normal range (i.e. 60-100 beats per minute for an otherwise healthy human subject 10 years of age or older, and for adults) and/or remains essentially constant upon administration of the Rhodospirillum rubrum cells to said human subject, preferably compared to said resting heart rate determined before the start of the first administration of the Rhodospirillum rubrum cells and/or during the subsequent further administration(s) of the Rhodospirillum rubrum cells.

An embodiment is the Rhodospirillum rubrum cells for use according to the invention or the non-therapeutic method of the invention, wherein the HDL-cholesterol concentration in the blood of the human subject remains unaltered or increases or is increased during the course of the administration of the Rhodospirillum rubrum cells, preferably once or twice daily administration, preferably compared to the HDL-cholesterol concentration determined before the start of the first administration of the Rhodospirillum rubrum cells and/or during the subsequent further administration(s) of the Rhodospirillum rubrum cells.

An aspect of the invention relates to a non-therapeutic method of treating or preventing a cardiovascular disease in a human subject, the method comprising administering to the human subject a composition comprising of or consisting of Rhodospirillum rubrum cells.

An aspect of the invention relates to a non-therapeutic method of treating or preventing atherosclerosis, dyslipidemia, arteriosclerosis, hypercholesterolemia, familial hypercholesterolemia, hyperlipidemia, homozygous sitosterolemia, an LDL-cholesterol plasma level of at least 70 mg/dL, an LDL-cholesterol plasma level of at least 100 mg/dL, an LDL-cholesterol plasma level of at least 140 mg/dL, an LDL-cholesterol plasma level of at least 200 mg/dL, a total plasma cholesterol level of at least 200 mg/dL, a total plasma cholesterol level of 5.0 mM-8.0 mM, a plasma Lp(a) level of at least 14 mg/dL, ischemia, in a human subject, the method comprising administering to the human subject a composition comprising of or consisting of Rhodospirillum rubrum cells.

An embodiment is the non-therapeutic method according to the invention, wherein the administration to the human subject of the composition comprising of or consisting of Rhodospirillum rubrum cells inhibits absorption of cholesterol from the intestine of the human subject, such that LDL-cholesterol concentration in the blood of said human subject decreases or is decreased, and preferably such that HDL-cholesterol concentration in the blood of the human subject remains unaltered or increases or is increased.

Embodiments are the Rhodospirillum rubrum cells for use according to the invention or the non-therapeutic method according to the invention, wherein the Rhodospirillum rubrum cells are administered to a human subject having an LDL-cholesterol level in plasma of at least 1.8 mmol/L (70 mg/dL), or at least 2.59 mmol/L (100 mg/dL), or at least 3.34 mmol/L (129 mg/dL), or at least 4.0 mmol/L, such as at least 5.2 mmol/L (200 mg/dL) or between 5.0 mM and 8.0 mM.

An aspect of the invention relates to an LDL-cholesterol-lowering method, a LDL-cholesterol-lowering compound and LDL-cholesterol-lowering composition with an improved safety profile, specifically for safe and efficacious use in the lowering of plasma LDL-cholesterol concentration in human subjects in need thereof, wherein the serum level of at least one health-related parameter selected from ALT, AST, γGT, creatinine, NT-ProBNP, vWF, c-Troponin T, (plasma) glucose, hsCRP remains within standard normal range and/or wherein SBP or DBP or both stay within standard normal range and/or wherein the resting heart rate stays within standard normal range in said human subject, i.e. 60-100 bpm.

An embodiment is the LDL-cholesterol-lowering method, a LDL-cholesterol-lowering compound and LDL-cholesterol-lowering composition according to the invention wherein the serum level of two or more of ALT, AST, γGT, creatinine, NT-ProBNP, vWF, c-Troponin T, plasma glucose, hsCRP remains within standard normal range and/or wherein SBP or DBP or both stay within standard normal range and/or wherein the resting heart rate stays within standard normal range, in said human subject when the compound or composition is administered to a human patient in need thereof, preferably the serum level of all of ALT, AST, γGT, creatinine, NT-ProBNP, vWF, c-Troponin T, plasma glucose, hsCRP, remains within standard normal range, and/or SBP or DBP or both stay within standard normal range and/or the resting heart rate stays within standard normal range in said human subject.

An aspect of the invention relates to an LDL-cholesterol-lowering method, a LDL-cholesterol-lowering compound and an LDL-cholesterol-lowering composition with an improved safety profile, specifically for safe and efficacious use in the lowering of plasma LDL-cholesterol concentration in human subjects in need thereof, wherein the serum level of at least one health-related parameter selected from ALT, AST, γGT, creatinine, NT-ProBNP, vWF, c-Troponin T, plasma glucose, hsCRP remains essentially unaltered and/or wherein SBP or DBP or both remain essentially unaltered and/or wherein the resting heart rate remains essentially unaltered, in said human subject, when the compound or composition is administered to a human patient in need thereof.

An embodiment is the LDL-cholesterol-lowering method, a LDL-cholesterol-lowering compound and LDL-cholesterol-lowering composition according to the invention wherein the serum level of two or more of ALT, AST, γGT, creatinine, NT-ProBNP, vWF, c-Troponin T, plasma glucose, hsCRP remains essentially unaltered and/or wherein SBP or DBP or both remain essentially unaltered and/or wherein the resting heart rate remains essentially unaltered, in said human subject, when the compound or composition is administered to a human patient in need thereof, preferably the serum level of all of ALT, AST, γGT, creatinine, NT-ProBNP, vWF, c-Troponin T, plasma glucose, hsCRP remains essentially unaltered and/or preferably SBP or DBP or both remain essentially unaltered and/or preferably resting heart rate remains essentially unaltered, in said human subject.

The inventors are the first to demonstrate an LDL-cholesterol lowering effect in the blood of human subjects to whom Rhodospirillum rubrum cells are administered, as established by conducting a clinical trial according to the gold standard, i.e. a double blind randomized controlled clinical trial with healthy human subjects having a total blood cholesterol concentration at the start of treatment of between 5.0 mmol/L and 8.0 mmol/L. The inventors are also the first to demonstrate an LDL-cholesterol lowering effect in the blood of human subjects to whom Rhodospirillum rubrum cells are administered, while the serum level of at least one health-related parameter selected from ALT, AST, γGT, creatinine, NT-ProBNP, vWF, c-Troponin T, plasma glucose, hsCRP remained essentially unaltered, while SBP and DBP remained essentially unaltered and resting heart rate remained essentially unaltered, in said human subject, as established with healthy human subjects having a total blood cholesterol concentration at the start of treatment of between 5.0 mmol/L and 8.0 mmol/L. The inventors furthermore established that the LDL-cholesterol lowering effect is the result and the consequence of inhibited absorption of cholesterol from the intestine in the human subjects. Thus, dietary cholesterol and biliary cholesterol are absorbed and transferred to a lower extent from the intestine and into the circulation when the human subjects (orally) take Rhodospirillum rubrum cells. The Rhodospirillum rubrum cells are dried Rhodospirillum rubrum cells obtained by subjecting freshly cultured Rhodospirillum rubrum cells to refractive drying using a refraction dryer. The human subjects to whom the Rhodospirillum rubrum cells were administered did not suffer from any side effect or adverse reaction or adverse event that could have otherwise been attributed or related to the daily intake of the Rhodospirillum rubrum cells. The serum levels for ALT, AST, γGT, creatinine, NT-ProBNP, vWF, c-Troponin T, plasma glucose, hsCRP remained essentially unaltered for the human subjects to whom the R. rubrum cells were administered. Furthermore, the SBP and the DBP remained essentially unaltered and stayed within normal, healthy boundaries, for the human subjects to whom the R. rubrum cells were administered in the conducted Phase 2 clinical trial. Additionally, resting heart rate also remained essentially unaltered and stayed within the normal range for the healthy human subjects included in the phase 2 clinical trial. Administering the Rhodospirillum rubrum cells to human subjects is thus free of accompanying adverse events and side effects. Daily intake of Rhodospirillum rubrum cells by the (healthy) human subjects resulted in a dose-dependent and linear decrease of the total cholesterol level in the blood, which decrease was fully relating to a decreased concentration of LDL-cholesterol (‘bad’ cholesterol). The total cholesterol/HDL-cholesterol ratio (TC/HDL ratio) decreased with the intake of Rhodospirillum rubrum cells, which indicates that the total cholesterol is made up of more HDL cholesterol and less LDL cholesterol than before intake of the R. rubrum cells. The levels of HDL-cholesterol did not alter, similar to the levels for ALT, AST, γGT, creatinine, NT-ProBNP, vWF, c-Troponin T, plasma glucose, hsCRP, and similar to the measured values for SBP and DBP and resting heart rate. Thus, it is concluded that the decrease of the TC/HDL ratio is attributed to the decrease in LDL-cholesterol.

The plasma LDL-cholesterol concentration is dictated partly by the efficiency of intestinal cholesterol absorption. The inventors established that twice daily dosing of R. rubrum cells to human subjects resulted in a decrease of the blood LDL-cholesterol concentration whereas HDL-cholesterol concentration remained unaltered and whereas serum levels for ALT, AST, γGT, creatinine, NT-ProBNP, vWF, c-Troponin T, plasma glucose, hsCRP remained essentially unaltered, whereas the SBP and the DBP remained essentially within healthy (standard normal) ranges and remained essentially unaltered and whereas resting heart rate remained essentially unaltered and within normal ranges during the clinical trial conducted, wherein the daily intake of the R. rubrum cells (gelatin capsules containing dried cells obtained with refractive drying) was without any adverse reaction, side effects, complaints, cumbersome consumption (hampered swallowing, etc.). The R. rubrum cells exert the LDL-cholesterol lowering activity by preventing the absorption of cholesterol from the intestine. The inventors established that compounds in the active fraction of fractioned freshly harvested R. rubrum cells are carotenoids rhodovibrin, 1-hydroxyspirilloxanthin, 3,4-didehydro-rhodopin, chloroxanthin, rhodopin, spirilloxanthin, and 3,4-dihydro-spirilloxanthin, (ubi)quinones and (ubi)quinoles ubiquinol-10, ubiquinone-9, ubiquinone-10, and rhodoquinone-10, and the bacteriopheophytins a, geranylgeranyl bacteriopheophytin a, and phytyl derivative of bacteriopheophytin a. The same as established for the mode of action of ezetimibe, R. rubrum cells also inhibit absorption of non-cholesterol sterols campesterol and sitosterol from the intestine, as confirmed by decreased levels in the blood of human subjects treated with R. rubrum cells. Common adverse drug reactions (≥1% of patients) associated with ezetimibe therapy include headache and/or diarrhea. Infrequent adverse effects (0.1-1% of patients) include myalgia and/or raised liver function test (aminotransferase (or alanine aminotransferase) versus aspartate aminotransferase ratio; ALT/AST) results. Rarely (<0.1% of patients), hypersensitivity reactions (rash, angioedema) or myopathy occurs. Cases of muscle problems (myalgia and rhabdomyolysis) have been reported and are included as warnings on the label for ezetimibe. Combination therapy using a statin and the cholesterol absorption inhibitor ezetimibe represents an approach to the treatment of hypercholesterolemia in the general population. Since R. rubrum therapy is not associated with any adverse reaction, R. rubrum cells are suitable for replacement therapy when ezetimibe is considered, or are suitable for at least partial replacement therapy: the dose of ezetimibe is lowered while a dose of R. rubrum cells is added to the therapeutic regimen. The administration of R. rubrum cells is either as a food supplement, or as a food ingredient in a food stuff, or as an active composition in a pharmaceutical composition.

There is a strong relationship between coronary atherosclerosis and coronary events and plasma cholesterol concentrations. A detailed study of this relationship was the Multiple Risk Factor Intervention Trial (MRFIT) involving 361,662 men in the age range of 35-57 years. The data from this and similar trials, together with results of a study in 9021 urban Chinese, showed that in free-living populations there is essentially a linear relationship between the rate of death from CHD and the plasma total cholesterol concentration between the levels of 150 mg/dL and 300 mg/dL. Only at concentrations below about 150 mg/dL does the mortality from CHD begin to approach zero. In humans, the bulk of cholesterol in plasma is carried in LDL. The importance of elevated plasma LDL-cholesterol levels as a risk factor for CHD is apparent not only from epidemiologic studies, but also from the results of extensive clinical trials involving the use of lipid-lowering drugs. Statins are amongst the most widely used cholesterol lowering drugs. However, a significant proportion of the human subjects receiving statin treatment continue to have plasma cholesterol levels that are above the range at which the incidence of CHD approaches zero.

The cholesterol carried in LDL, like all cholesterol throughout the body, originates ultimately from the diet and from de novo synthesis within the tissues. In adult human subjects consuming a typical Western diet, about 1100 mg of cholesterol enters the body pools daily. About one quarter of the 1100 mg cholesterol (300 mg) comes from the diet. The remainder (800 mg) is synthesized by the body. The bulk cholesterol synthesis is in the major extrahepatic organs. Under steady state conditions, the amount of cholesterol excreted by the body equals the input from diet and synthesis so that there is no net accumulation of cholesterol in the body other than the tiny amount that is progressively sequestered in atherosclerotic plaques, as the case may be. The balance between cholesterol input and cholesterol output involves the interplay of multiple complex biosynthetic, transport, catabolic, and excretory pathways. Essentially, this balance is achieved because each day about 800 mg of cholesterol is returned from the periphery to the liver for excretion in bile, either as unmetabolized cholesterol, or as bile acids, which are the principal degradation products of cholesterol.

Within the lumen of the small intestine biliary cholesterol mixes with dietary cholesterol. A portion of this luminal cholesterol is subsequently absorbed across the intestinal mucosa into the lymph and is carried into the plasma in chylomicrons. In the circulation, the chylomicrons are hydrolyzed by lipoprotein lipase to form remnant particles that are then rapidly taken up by the liver. This enterohepatic movement of biliary and dietary cholesterol is of fundamental importance in dictating cholesterol balance across the whole body, and ultimately of the concentration of LDL-cholesterol in the plasma. Each day about 1000 mg of biliary cholesterol enters the lumen of the small intestine compared with about 300 mg from the diet. Therefore, even if dietary cholesterol intake were halved, this would only marginally decrease the amount of cholesterol within the intestinal lumen that could potentially be absorbed. Such dietary modification would thus likely be of only modest benefit in reducing the plasma LDL-cholesterol concentration. However, if the absorption efficiency of biliary and dietary cholesterol is reduced pharmacologically or by a food supplement to a certain extent, then the magnitude of the reduction in the amount of cholesterol entering the body, and the magnitude of the reduction in plasma LDL-cholesterol levels, is more significant.

Although the liver is not a major site of de novo cholesterol synthesis, it plays a central role in regulating plasma LDL-cholesterol levels. This occurs because the liver is the principal site for both the production of LDL as well as its receptor-mediated clearance from the plasma. Cholesterol entering the liver from the uptake of chylomicrons and plasma lipoproteins, together with cholesterol synthesized within the hepatocyte, mixes in a common regulatory pool. Sterol from this pool is utilized in several pathways, one of which involves the production of nascent very low-density lipoproteins, which are the precursors of most of the LDL in the circulation. The liver is also quantitatively the most important organ for removing LDL from the plasma via the LDLR mediated pathway. Thus, the plasma LDL-C concentration can be modulated over a broad range by changing the rate of production and/or clearance of LDL by the liver. Both of these facets of hepatic LDL metabolism are highly responsive to changes in the amount of cholesterol that is absorbed from the intestinal lumen and carried to the liver in the remnants of chylomicrons. Thus, increasing the delivery of chylomicron cholesterol to the liver drives LDL production, suppresses LDLR activity, and raises plasma LDL-cholesterol levels, whereas inhibiting cholesterol absorption (from the intestine) has the reverse effect. Cholesterol absorption from the intestine occurs primarily in the duodenum and proximal jejunum. The absorption process is largely specific for cholesterol because plant sterols, although structurally similar to cholesterol, are generally absorbed either poorly or not at all. In humans consuming a typical Western diet, only about one quarter of the cholesterol entering the lumen of the small bowel is from the diet; the bulk of cholesterol in the lumen comes directly from the bile and cells shed from the intestinal epithelium. There are two main phases of cholesterol absorption. The first takes place in the lumen and involves digestion and hydrolysis of dietary lipids followed by solubilization of cholesterol in mixed micelles containing bile acid and phospholipids. This solubilization facilitates the movement of cholesterol from the bulk phase of the lumen to the surface of the enterocyte. In the second phase, cholesterol crosses the mucosal cell membrane by facilitated diffusion under influence of the Niemann-Pick C1 Like 1 protein (NPC1L1). NPC1L1 is an essential protein in the intestinal cholesterol uptake and absorption process. NPC1L1 localizes to the brush border membrane of absorptive enterocytes in the small intestine. Intestinal expression of NPC1L1 is down regulated by diets containing high levels of cholesterol and triglycerides. Research over the past 30-50 years has led to synthetic and natural products that block cholesterol absorption either by disruption of the micellar solubilization step, as appears to be the case with plant sterols and their derivatives, or by inhibition of one of the steps involved in the intracellular phase of the absorption process, as exemplified by inhibitors of the esterifying enzyme, acyl coenzyme A: cholesterol acyltransferase. Although many of these agents exert a marked cholesterol-lowering action in animal models, most have proved to be less efficacious in humans. Those inhibitors of cholesterol absorption that are moderately effective in humans usually need to be taken in gram quantities daily, which in some cases limits patient tolerance and compliance. The inventors now provide for an LDL-cholesterol lowering food supplement or (pharmaceutical) composition, relating to dried R. rubrum cells, wherein the use of the R. rubrum cells in human subjects was established without any side effects or adverse reactions, and wherein the amount of active ingredient(s) expressed as a weight percentage was at most about 1.5% of the dry mass of the R. rubrum cells, accumulating to 3.75 mg-15 mg active ingredient(s) in R. rubrum whole cells administered daily to the human subjects, divided over two equal doses daily.

Embodiments are the Rhodospirillum rubrum cells for use according to the invention, or the non-therapeutic method according to the invention, wherein Rhodospirillum rubrum cells are administered to a human subject to whom at least one active pharmaceutical ingredient is (co-)administered, such as at least one active pharmaceutical ingredient selected from a statin, niacin, fenofibrate, ezetimibe, colesevelam, mipomersen, lomitapide, a PCSK9 inhibitor, alirocumab, evolocumab, ETC-1002, a CETP inhibitor, anacetrapib, evacetrapib, WAY-252623, a blood-pressure lowering compound, hydrochlorothiazide, preferably a statin such as atorvastatin, fluvastatin, pravastatin, rosuvastatin, simvastatin, lovastatin and/or ezetimibe. When for example a statin and/or ezetimibe was previously administered to a human subject at the start of administering the Rhodospirillum rubrum cells, the Rhodospirillum rubrum cells either replace the statin and/or the ezetimibe, or these active pharmaceutical compounds are used at a lower dose when co-administered with the Rhodospirillum rubrum cells, compared to the dose(s) applied when Rhodospirillum rubrum cells was not administered to the human subject.

Embodiments are the Rhodospirillum rubrum cells for use according to the invention or the non-therapeutic method according to the invention, wherein administering the Rhodospirillum rubrum cells to a human subject results in a decrease of the plasma LDL-cholesterol concentration in said subject with at least 1%, preferably at least 5%, more preferably at least 6%, more preferably at least 10%, most preferably at least 20%, based on the plasma LDL-cholesterol concentration prior to the administration of the Rhodospirillum rubrum cells to said human subject. Preferably, administering the Rhodospirillum rubrum cells to a human subject results in a decrease of the plasma LDL-cholesterol concentration in said subject, while the concentration of HDL-cholesterol essentially remains unaltered or increases. Preferably, administering the Rhodospirillum rubrum cells to a human subject results in a decrease of the plasma LDL-cholesterol concentration in said subject, while the concentration of any one or more of ALT, AST, γGT, creatinine, NT-ProBNP, vWF, c-Troponin T, plasma glucose, hsCRP in the serum (plasma for glucose) of said subject essentially remains unaltered or stays within (standard) normal and healthy values, preferably for all these parameters ALT, AST, γGT, creatinine, NT-ProBNP, vWF, c-Troponin T, plasma glucose, hsCRP in the serum (plasma for glucose) of said subject, the concentrations essentially remains unaltered or within (standard normal) healthy boundaries. Also preferred is administering the Rhodospirillum rubrum cells to a human subject, resulting in a decrease of the plasma LDL-cholesterol concentration in said subject, while one or both of SBP and DBP remain essentially unaltered for said subject, or stay within (standard) normal, healthy boundaries. Also preferred is administering the Rhodospirillum rubrum cells to a human subject, resulting in a decrease of the plasma LDL-cholesterol concentration in said subject, while resting heart rate remains essentially unaltered for said subject, or stays within (standard) normal, healthy boundaries. Preferably, the human subject is an otherwise healthy subject, for example a human subject having borderline plasma cholesterol or plasma LDL-cholesterol level such as 5 mM-8 mM. More preferably, administering the Rhodospirillum rubrum cells to a human subject results in a decrease of the plasma LDL-cholesterol concentration in said subject, while the concentration in the serum of said subject essentially remains within standard normal values for ALT, AST, γGT, creatinine, NT-ProBNP, vWF, c-Troponin T, plasma glucose, hsCRP, while the SBP and the DBP either remains essentially unaltered or stays within healthy and standard normal boundaries and while the resting heart rate remains essentially unaltered or stays within the healthy and standard normal range.

Embodiments are the Rhodospirillum rubrum cells for use according to the invention or the non-therapeutic method according to the invention, wherein the Rhodospirillum rubrum cells are formulated as a granulate, preferably a granulate provided in a capsule such as a gelatin capsule or in a sachet. Preferred are dried Rhodospirillum rubrum cell granules obtained by subjecting cells, preferably freshly cultured cells to drying such as refractive drying at 55° C.-100° C., preferably at 60° C.-100° C., more preferably at 80° C.-100° C., using a refraction dryer, such as 55° C.-70° C. or 58° C.-65° C.

Embodiments are the Rhodospirillum rubrum cells for use according to the invention or the non-therapeutic method according to the invention, wherein a daily dose of Rhodospirillum rubrum cells to be administered to a human subject is at least 1.0 g, such as at least 5.0 g, preferably 1.0-100 g, more preferably 1.0-50 g, most preferably 5.0-20 g. Preferred are the Rhodospirillum rubrum cells for use according to the invention or the non-therapeutic method according to the invention, wherein the Rhodospirillum rubrum cells are dosed to provide an amount of between 0.1 g and 50 g of Rhodospirillum rubrum cells per day.

Embodiments are the Rhodospirillum rubrum cells for use according to the invention or the non-therapeutic method according to the invention, wherein a daily dose of Rhodospirillum rubrum cells to be administered to a human subject are two half-daily doses each comprising half of the daily dose, wherein preferably a first half-daily dose is administered 1-2 hours before lunch, 1-2 hours after lunch or during lunch taken by the human subject, and a second half-daily dose is administered 1-2 hours before dinner, 1-2 hours after dinner or during dinner taken by the human subject, preferably during lunch and during dinner.

An aspect of the invention relates to a pharmaceutical composition comprising a pharmaceutically effective amount of Rhodospirillum rubrum cells and optionally a pharmaceutically acceptable excipient, for use in a method for the lowering of LDL-cholesterol in blood plasma of a human subject.

An embodiment is the pharmaceutical composition for use according to the invention, wherein the concentration of at least one of ALT, AST, γGT, creatinine, NT-ProBNP, vWF, c-Troponin T, plasma glucose and hsCRP remains within a standard normal range and/or remains essentially constant in the serum and/or plasma of the human subject upon administration of the pharmaceutical composition to said human subject, preferably all the concentrations remain essentially constant and/or remain within the standard normal range, preferably compared to the concentration(s) determined for said at least one of ALT, AST, γGT, creatinine, NT-ProBNP, vWF, c-Troponin T, plasma glucose and hsCRP before the start of the first administration of the pharmaceutical composition and/or during the subsequent one or more further administrations of the pharmaceutical composition.

An embodiment is the pharmaceutical composition for use according to the invention, wherein diastolic blood pressure and/or systolic blood pressure of the human subject remain(s) within a standard normal range and/or remain(s) essentially constant upon administration of the pharmaceutical composition to said human subject, preferably compared to said blood pressure(s) determined before the start of the first administration of the pharmaceutical composition and/or during the subsequent further administration(s) of the pharmaceutical composition.

An embodiment is the pharmaceutical composition for use according to the invention, wherein the resting heart rate of the human subject remains within a standard normal range and/or remains essentially constant upon administration of the pharmaceutical composition to said human subject, preferably compared to said resting heart rate determined before the start of the first administration of the pharmaceutical composition and/or during the subsequent further administration(s) of the pharmaceutical composition.

An aspect of the invention relates to a pharmaceutical composition comprising a pharmaceutically effective amount of Rhodospirillum rubrum cells and optionally a pharmaceutically acceptable excipient, for use in a method for the treatment or prophylaxis of any one or more of cardiovascular disease, atherosclerosis, dyslipidemia, arteriosclerosis, hypercholesterolemia, familial hypercholesterolemia, hyperlipidemia, homozygous sitosterolemia, an LDL-cholesterol plasma level of at least 70 mg/dL, an LDL-cholesterol plasma level of at least 100 mg/dL, an LDL-cholesterol plasma level of at least 140 mg/dL, an LDL-cholesterol plasma level of at least 200 mg/dL, a total plasma cholesterol level of at least 200 mg/dL, a total plasma concentration in the blood of at least 5.0 mM such as between 5.0 mM and 8.0 mM, an Lp(a) level of at least 14 mg/dL, inflammation, inflammatory disease, ischemia, infection.

An embodiment is the pharmaceutical composition for use according to the invention, wherein the Rhodospirillum rubrum cells are administered to the human subject as replacement therapy such as therapy replacing a statin and/or ezetimibe, or are administered to the human subject in combination with a lower dose of (a) LDL-cholesterol lowering pharmaceutical compound(s) administered to the human subject than the dose of such (a) LDL-cholesterol lowering pharmaceutical compound(s) administered to the human subject before administration of Rhodospirillum rubrum cells started, wherein the LDL-cholesterol lowering pharmaceutical compound(s) preferably is/are a statin, ezetimibe.

An embodiment is the pharmaceutical composition for use according to the invention, wherein the Rhodospirillum rubrum cells are the Rhodospirillum rubrum cells of any of the embodiments of the invention.

An aspect of the invention relates to a food supplement with cholesterol-lowering properties when orally ingested by a human subject, comprising Rhodospirillum rubrum cells, wherein the Rhodospirillum rubrum cells are dried Rhodospirillum rubrum cell granules obtained by subjecting cells such as freshly cultured cells to drying, preferably refractive drying at 55° C.-100° C., preferably at 60° C.-100° C., more preferably at 80° C.-100° C., such as 55° C.-76° C., 57° C.-72° C., 59° C.-68° C. or 60° C.-65° C.

An aspect of the invention relates to a foodstuff comprising a food supplement according to the invention.

Embodiments are the food supplement of the invention or the foodstuff of the invention, wherein the Rhodospirillum rubrum cells are the Rhodospirillum rubrum cells of any of the embodiments of the invention.

An aspect of the invention relates to a kit comprising: (i) a container containing Rhodospirillum rubrum cells of any one of the embodiments of the invention, preferably dried Rhodospirillum rubrum cell granules, or the food supplement of the invention, or the foodstuff of the invention; (ii) instructions for use by a human subject; and (iii) optionally at least one pharmaceutical composition comprising any of the active pharmaceutical ingredients of embodiments of the invention, preferably a statin and/or ezetimibe; and (iv) optionally a container with a liquid, preferably water or a drink, the liquid for mixing the provided Rhodospirillum rubrum cells in the container before intake by a human subject, or the liquid for sequential or concurrent intake with the Rhodospirillum rubrum cells by a human subject.

An aspect of the invention relates to a kit comprising:
a container containing Rhodospirillum rubrum cells of the invention or the pharmaceutical composition for use according to the invention or the food supplement of the invention, or the foodstuff of the invention;
instructions for use by a human subject; and
optionally a container with a liquid, preferably water or a drink, the liquid for mixing the provided Rhodospirillum rubrum cells in the container before intake by a human subject, or the liquid for sequential or concurrent intake with the Rhodospirillum rubrum cells by a human subject.

A further aspect of the invention relates to the Rhodospirillum rubrum cells according to the invention, a food supplement comprising the Rhodospirillum rubrum cells, a food stuff comprising the Rhodospirillum rubrum cells, or a pharmaceutical composition comprising the Rhodospirillum rubrum cells, wherein the serum concentration for at least one (health-related) parameter selected from ALT, AST, γGT, creatinine, NT-ProBNP, vWF, c-Troponin T, plasma glucose and hsCRP remains essentially unaltered or remains within the (standard) normal range and/or wherein SBP and DBP remain essentially unaltered or within the healthy normal boundaries and/or wherein resting heart rate remains essentially unaltered or remains within the (standard) normal range, after administration of the Rhodospirillum rubrum cells to a human subject in need thereof, preferably the serum (plasma) values for all of said health-related parameters, blood pressures and resting heart rate remain in the (standard) normal range and/or remain essentially unaltered when the values and blood pressures are determined before start of administering the cells to the human subject and during the course of daily administration of the cells, and compared, and/or when the values are determined at a first day during the course of daily administration of the cells to a human subject in need thereof and at a second day during said course of daily administration, and compared. The (standard) normal (healthy) range for the serum concentrations (plasma concentration for glucose) of the parameters as defined herein is between 8 U/L and 33 U/L for AST, between 4 U/L and 36 U/L for ALT, between 5 U/L and 40 U/L for γGT, between about 61.9 μmol/L and 114.9 μmol/L for men and between 53 μmol/L and 97.2 μmol/L for women for creatinine, less than 125 μg/mL for human subjects below the age of 75 and less than 450 μg/mL for human subjects above the age of 75 for NT-ProBNP, between 50 U/L and 200 U/dL for vWF, less than 52 ng/mL for c-Troponin T, between 4.0 mmol/L and 5.9 mmol/L when fasting for plasma glucose and less than 3.0 mg/L for hsCRP. The standard normal range for the blood pressures is between 90 mmHg and 139 mmHG for SBP, and between 60 mmHg and 89 mmHG for DBP. The standard normal range for resting heart rate is between 60 to 100 beats per minute, for otherwise healthy human subjects, i.e. adults or subjects 10 years of age and older.

### SHORT DESCRIPTION OF DRAWINGS

The present invention will be discussed in more detail below, with reference to the attached drawings and figures, in which:

FIG. 1 displays the scope of FIG. 2 of WO2004/052380A1, displaying the lipoprotein pattern in plasma of Wistar rats that were fed a normal chow diet (‘Controls’), and from Wistar rats that were fed a chow diet containing 10% (w/w) R. rubrum (‘10% R.rub’), as explained in detail in Example 5 of WO 2004/052380 A1. Under influence of consumed R. rubrum cells the plasma LDL-cholesterol levels in the rats decreased, whereas the plasma HDL-cholesterol levels remained essentially unaltered.

FIG. 2 displays the scope of FIG. 5 of WO2004/052380A1, showing the lipoprotein pattern in plasma of C57Bl/6 mice that were fed a hypercholesterolaemic “Western-type” diet, and a hypercholesterolaemic “Western-type” diet containing 10% (w/w) R. rubrum, as outlined in detail in Example 7 of WO 2004/052380 A1. Under influence of consumed R. rubrum cells the plasma LDL-cholesterol levels in the mice decreased, whereas the plasma HDL-cholesterol levels remained essentially unaltered.

FIG. 3 displays the scope of FIG. 1 of WO2004/052380A1, showing the total cholesterol content in plasma of male Wistar rats that were fed a rat chow (“controls”) or the rat chow containing in addition 10% freeze-dried R. rubrum cells based on the total weight of the rat chow and R. rubrum cells, as outlined in detail in Example 5 of WO 2004/052380 A1. Under influence of consumed R. rubrum cells the total cholesterol content in plasma of the male Wistar rats decreased, compared to the control rats that were not fed with the R. rubrum cells.

FIG. 4 shows the scope of FIG. 9 of WO2004/052380A1, showing the effect of 5% (w/w) and 10% (w/w) of R. rubrum in a “Western-type” diet of mice on plasma cholesterol levels and on β-sitosterol levels after two weeks being fed, compared to a group of control mice fed with the same diet lacking the R. rubrum cells (data shown are mean±s.d.).

FIG. 5 shows the scope of FIG. 2 of international patent application WO2018/229223A1 by the same inventors, showing the Maldi TOF analysis of a petroleum-ether extract “l.1” of Rhodospirillum rubrum cells.

FIG. 6 shows the scope of FIG. 1 of international patent application WO2018/229223A1, showing the plasma cholesterol lowering effect with regard to LDL-cholesterol in an in vivo mouse model, while HDL-cholesterol levels and total cholesterol levels essentially remain unaltered upon treatment of the mice. Mice were fed control feed (‘control’) or feed enriched with an extract of bacterium Rhodospirillum rubrum comprising a carotenoid, a quinone, an ubiquinone, an ubiquinol, an ubiquinone and a bacteriopheophytin (‘Extract l.1/Fraction l.1’).

FIG. 7 shows the essentially equal weekly food intake of mice that were first fed regular high-fat feed and subsequently subjected to a comparative study in which the ‘Control’ group of mice continued with being fed with the regular high-fat diet supplemented with 10% regular diet based on the total weight of the feed, whereas the ‘Treated’ group of mice were fed with the regular high-fat diet supplemented with 10% R. rubrum cells based on the total weight of the feed.

FIG. 8 shows the results of the comparative study referred to in the outline of FIG. 7, when total plasma cholesterol levels in the mice was considered, wherein in the study the ‘Control’ group of mice were fed regular high-fat diet supplemented with 10% regular diet based on the total weight of the feed, and the ‘Treated’ group of mice were fed with the regular high-fat diet supplemented with 10% R. rubrum cells based on the total weight of the feed.

FIG. 9 shows the experimental design of a randomized, double-blind, placebo-controlled clinical trial according to the gold standard for clinical trials, including 82 healthy male human subjects with a slightly elevated fasting serum total cholesterol concentration (between 5.0-8.0 mmol/l; 193-309 mg cholesterol per dL), wherein the subjects consumed a capsule containing 0.25 gram dried R. rubrum cells per day (two capsules comprising 0,125 gram, daily), or 0.50 gram, or 1.0 gram, or placebo (no R. rubrum cells in the diet) (n=20 per group).

FIG. 10 shows the experimental design of a second randomized, double-blind, placebo-controlled clinical trial according to the gold standard for clinical trials, wherein healthy male human subjects with a slightly elevated fasting serum total cholesterol concentration (between 5.0-8.0 mmol/l; 193-309 mg cholesterol per dL), consume capsules containing 1.0, 2.0, 3.0, 4.0 and 5.0 gram dried R. rubrum cells per day or placebo.

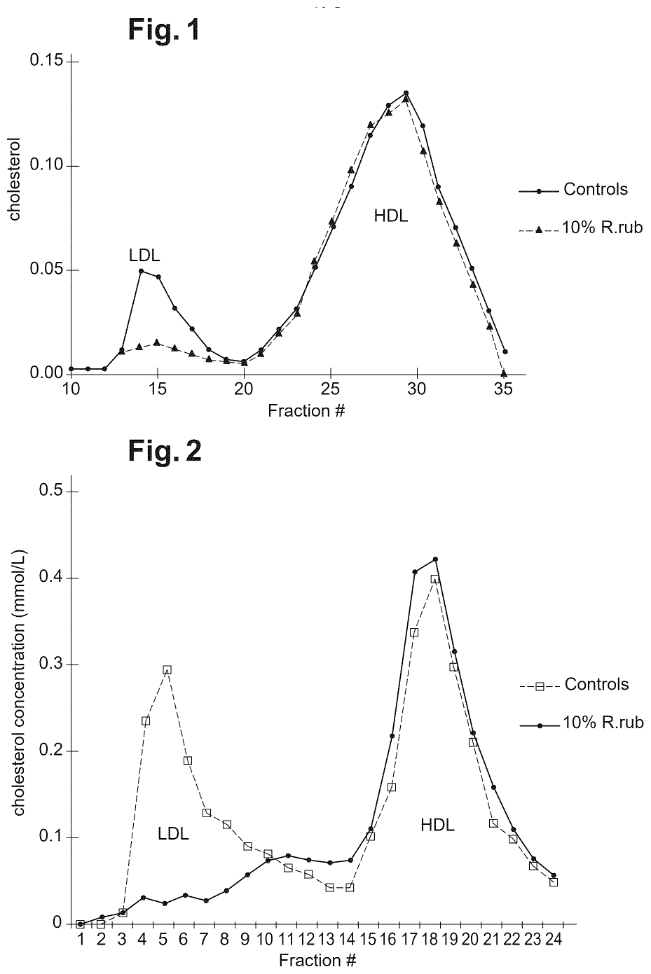

### DEFINITIONS AND STANDARDS

The terms first, second, third and the like in the description and in the claims, are used for distinguishing between similar elements and not necessarily for describing a sequential or chronological order. The terms are interchangeable under appropriate circumstances and the embodiments of the invention can operate in other sequences than described or illustrated herein.

The embodiments of the invention described herein can operate in combination and cooperation, unless specified otherwise.

Furthermore, the various embodiments, although referred to as “preferred” or “e.g.” or “for example” or “in particular” are to be construed as exemplary manners in which the invention may be implemented rather than as limiting the scope of the invention.

The term “comprising”, used in the claims, should not be interpreted as being restricted to the elements or steps listed thereafter; it does not exclude other elements or steps. It needs to be interpreted as specifying the presence of the stated features, integers, steps or components as referred to, but does not preclude the presence or addition of one or more other features, integers, steps or components, or groups thereof. Thus, the scope of the expression “a composition comprising A and B” should not be limited to a composition consisting only of compounds A and B, rather with respect to the present invention, the only enumerated compounds of the composition are A and B, and further the claim should be interpreted as including equivalents of those compounds.

A cholesterol-lowering property is herein defined as the capability of a compound or a composition, such as dried R. rubrum cells, or oven-dried R. rubrum cells obtained with refractive drying (dried in a refraction dryer known in the art, at 55-100° C., preferably at 60-100° C., more preferably at 80-100° C., such as 53° C.-73° C., 56° C.-68° C., 58° C.-63° C.), a pharmaceutical composition, a preparation, a food supplement or a foodstuff or a feed (product) for an animal, when (orally) administered to the body of a subject, such as an animal subject, for example a (healthy) human subject in need thereof, to lower the cholesterol, or LDL-cholesterol, level of the blood or the plasma or the serum of said subject. Methods for measuring the level of cholesterol in blood and plasma and serum are known to the skilled person.

Throughout the specification the term “petroleum ether with a boiling point of between 60° C. and 80° C.” has its regular scientific meaning and here refers to a petroleum fraction consisting of aliphatic hydrocarbons and boiling in the range of between 60° C. and 80° C.

The term “Acute kidney injury” has its regular scientific meaning and is defined by the “Kidney Disease: Improving Global Outcomes” (KDIGO) 2012 guidelines as 20.3 mg/dL absolute rise in creatinine from baseline within 48 hours.

The term “LDL-cholesterol level” and the term “LDL-cholesterol concentration” have their regular scientific meaning throughout the description and in the claims, and here refer to the amount of cholesterol in mmol/L or in mg/dL, that is associated with and transported by LDL in the body of the subject, e.g. a (healthy) human subject. The LDL-cholesterol level is for example measured directly, as outlined in the Examples section. Where indicated, the LDL-cholesterol concentration is calculated from the measured total cholesterol concentration, the measured HDL-cholesterol concentration and the measured tri-acyl glycerol concentration in a sample such as a plasma sample of a human subject, and based on these measured data, the LDL-cholesterol level is calculated.

A “Phase IIa clinical trial” has its regular scientific meaning and here refers to a clinical trial according to the gold standard for clinical trials with human subjects, including healthy human male subjects to whom an investigational product, here oven-dried Rhodospirillum rubrum bacterial cells (oven-dried R. rubrum cells obtained with refractive drying using a refraction dryer known in the art (at 55-100° C., preferably at 60-100° C., more preferably at 80-100° C., such as 56° C.-90° C., 58° C.-80° C., 60° C.-70° C. and 62° C.-68° C.)), or placebo is administered with the purpose of examining for the first time the safety of administering the bacterial cells to human subjects and for assessing LDL-cholesterol lowering effect of the dried Rhodospirillum rubrum in humans. In the Phase II trial, the bacterial cells are evaluated for their efficacy and safety in human subjects in need of reduction of plasma LDL-cholesterol levels, i.e. plasma LDL-cholesterol levels of at least 5.0 mM and at most 8.0 mM. The phase II trial is a placebo-controlled trial. The purpose of a Phase IIa trial is to evaluate short-term safety of the drug (or the food supplement), whereas drug efficacy is also monitored during such a trial.

The term “R. rubrum”, “R. rubrum cell” and “R. rubrum bacterial cell” have their regular scientific meaning and here refer to the gram negative photosynthesizing, red bacterium Rhodospirillum rubrum.

The term “homeostasis” has its regular scientific meaning in biology, referring to the state of steady internal physical and chemical conditions maintained by living systems. This dynamic state of equilibrium is the condition of optimal functioning for the organism and includes many variables, such as body temperature and fluid balance, being kept within certain pre-set limits (homeostatic range). Other variables include the pH of extracellular fluid, the concentrations of sodium, potassium and calcium ions, as well as that of the blood sugar level, and these need to be regulated despite changes in the environment, diet, or level of activity. Each of these variables is controlled by one or more regulators or homeostatic mechanisms, which together maintain life. In the context of the invention the term homeostasis i.e. the dynamic state of equilibrium, refers to the condition of optimal functioning for a subject such as a (healthy) human subject when plasma LDL-cholesterol levels are considered and/or total cholesterol levels are considered.

AST is an indicator of liver inflammation. An increased level of AST is often a sign of liver disease, but also indicates a heart attack, muscle disease or trauma, pancreatitis, and severe burns. According to MEDLINE® (official website of NIH-NLM (U.S. National Library of Medicine), USA.gov, Last Reviewed: Jun. 4, 2019; accessed on 30 Oct. 2019; www.nirn.nih.gov), the normal range for AST is between 8 to 33 U/L for a human subject. AST may be measured using a spectrophotometric assay. Preferably, the spectrophotometric assay is done on a Cobas 8000 (Roche Diagnostics System) (See Examples section, below).

ALT is commonly measured in liver function tests. ALT is also a common indicator of liver disease. Significantly elevated levels of ALT often suggest the existence of medical problems such as viral hepatitis, diabetes, congestive heart failure, liver damage, bile duct problems, infectious mononucleosis, or myopathy. ALT levels may fluctuate slightly over the course of the day, but according to MEDLINE® the normal range for a human is between 4 to 36 U/L. ALT may be measured using a spectrophotometric assay. Preferably, the spectrophotometric assay is done on a Cobas 8000 (Roche Diagnostics System) (See Examples section, below).

γGT is also a diagnostic marker for liver disease. Elevated serum γGT activity can be found in diseases of the liver, biliary system, and pancreas. It has additionally been found that slightly elevated levels of γGT are also an indicator of cardiovascular disease. Reference ranges for γGT in humans are between 5 to 40 U/L according to MEDLINE®. γGT may be measured using a spectrophotometric assay. Preferably, the spectrophotometric assay is done on a Cobas 8000 (Roche Diagnostics System) (See Examples section, below).

Creatinine is the most commonly used indicator of renal function. Elevated levels of creatinine may be due to a blocked urinary tract, kidney problem, dehydration or rhabdomyolysis (breakdown of muscle fibres). The typical human reference ranges, according to MEDLINE®, for serum creatinine are 0.7 mg/dL to 1.3 mg/dL (about 61.9 μmol/L to 114.9 μmol/L) for men and 0.6 mg/dL to 1.1 mg/dL (53 μmol/L to 97.2 μmol/L) for women. Creatinine may be measured using a spectrophotometric assay. Preferably, the spectrophotometric assay is done on a Cobas 8000 (Roche Diagnostics System) (See Examples section, below).

NT-ProBNP is used for screening and diagnosis of acute congestive heart failure and may be useful to establish prognosis in heart failure. NT-ProBNP is typically elevated in patients with a worse outcome in heart failure as well as patients with asymptomatic or symptomatic left ventricular dysfunction. Additionally, it is associated with coronary artery disease and myocardial ischemia. Acceptable values for NT-ProBNP change with age and there is no level of NT-ProBNP that accurately predicts all health problems associated with this marker. However, according to the handbook “Basic Skills in Interpreting Laboratory Data”, Chapter 10, page 220 (4^{th} Ed., Mary Lee, American Society of Health-System Pharmacists, Bethesda, Maryland (USA)), under the header “N-Terminal-ProBNP (NT-proBNP)”, it is stated that human subjects under the age of 75 years have a higher risk of congestive heart failure if their level of NT-ProBNP exceeds 125 μg/mL, while for human subjects over the age of 75 years this is more than 450 μg/mL. NT-ProBNP may be measured using an immunoassay. Preferably, the immunoassay is done on a Cobas 8000 (Roche Diagnostics System) (See Examples section, below).

vWF is a blood glycoprotein involved in haemostasis. Increased plasma levels of vWF have been observed in cardiovascular, neoplastic, and connective tissue diseases, which are presumed to arise from adverse changes to the endothelium. Recently, it has been discovered that elevated vWF is also a risk factor for coronary artery disease. The normal reference range for vWF in humans is between 50 and 200 U/dL according to the National Heart, Lung, and Blood Institute (USA.gov, “The Diagnosis, Evaluation and Management of von Willebrand Disease: Full Report” accessed on 4 November, www.nhlbi.nih.gov). vWF may be measured using ELISA.

c-Troponin T is especially useful in the laboratory diagnosis of heart attack because it is released into the blood-stream when damage to heart muscle occurs. According to Medscape (reference.medscape.com, point-of-care medical reference for physicians and healthcare professionals, partners with e.g. Albert Einstein Medical Center, Yale School of Medicine, Updated: 14 Jan. 2015, accessed on 4 Nov. 2019), the normal level for c-Troponin T is less than 14 ng/mL, however 14-52 ng/mL is borderline, while truly elevated levels of c-Troponin T start at more than 52 ng/mL. c-Troponin T may be assessed using an immunoassay. Preferably, the immunoassay is done on a Cobas 8000 (Roche Diagnostics System) (See Examples section, below).

Plasma glucose concentrations are a good indicator for several clinical abnormalities. Firstly, a high plasma glucose level may cause problems such as heart disease, cancer, kidney damage, eye damage and nerve damage. While a low plasma glucose causes lethargy, impaired mental functioning; irritability; shaking, twitching, weakness in arm and leg muscles; pale complexion; sweating; loss of consciousness. If low plasma glucose is not treated in time, serious consequences such as loss of consciousness, seizures and loss of organ function may occur. Normal plasma glucose levels in non-diabetic humans range between 4.0-5.9 mmol/L before a meal up to 7.8 mmol/mL after a meal according to the National Institute for Health and Care Excellence Public Health Guideline 38 (UK, www.nice.org.uk/guidance/ph38, last reviewed September 2017, accessed on 4 Nov. 2019).

hsCRP is mainly used as an indicator of inflammation. High circulating levels of CRP indicate the presence of a continuous systemic inflammation. It is also a predictor of liver failure, diabetes and the development of cardiovascular disease. In healthy adult humans, the normal concentrations of hsCRP varies. However, it has been found by the American Heart Association and U.S. Centers for Disease Control and Prevention (labtestsonline.org/tests/high-sensitivity-c-reactive-protein-hs-crp, last reviewed 23 Oct. 2018, accessed on 4 Nov. 2019) that individuals with less than 1.0 mg/L hsCRP have a low risk of cardiovascular disease, while people with over 3.0 mg/L have a high risk of cardiovascular disease. hsCRP may be measured by an immunoturbidimetric assay.

SBP and DBP are measures of predicting the risk of cardiovascular disease. Both high blood pressure and low blood pressure are associated with several serious conditions related to the circulatory system. The Task Force for the management of arterial hypertension of the European Society of Cardiology (ESC) and the European Society of Hypertension (ESH) (2018 ESC/ESH Guidelines for the management of arterial hypertension, www.eshonline.org/guidelines/arterial-hypertension, accessed on 4 Nov. 2019) has established a classification of blood pressure in relation to risk of hypertension. SBP that is considered optimal is lower than 120 mmHG, however up to a 139 mmHg is still considered to be normal for humans. DBP is optimally lower than 80 mmHG, however up to 89 mmHG it is still considered to be normal for humans. There is no accepted diagnostic standard for a “too” low blood pressure, but commonly it is accepted that a SBP of lower than 90 mmHG and a DBP of lower than 60 mmHG is too low for a human adult. Preferably, DBP and SBP are measured using an Omron M7 (Omron Healthcare Co. Ltd., Kyoto, Japan). See Examples section, below.

Resting heart rate is an indication of the health condition of the heart. A consistently higher or lower resting heart rate than usual for a human subject may indicate problems related to the heart. According to MEDLINE® a normal healthy resting heart rate lies between 60-100 bpm (beats per minute; otherwise healthy human adult, 10 years of age or older). However, what is considered “normal” for a single subject varies based on several factors, such as overall fitness and age. For instance, people that frequently exercise, or a professional athlete may have a resting heart rate as low as 40 bpm. Research has shown that a resting heart rate near the top of the 60-100 range can increase the risk of cardiovascular disease. A higher resting heart rate is also associated with lower physical fitness and higher blood pressure, body weight and levels of circulating blood fats. A resting heart rate of over 100 beats per minute is a condition called tachycardia, which indicates one or more of several diseases such a sepsis, hyperthyroidism, and cardiomyopathy. A resting heart rate of lower than 60 bpm, in general, is a condition called bradycardia. Bradycardia may indicate the presence of for instance hypothyroidism and heart block.

In summary: The standard normal (healthy) range as defined herein is between 8 U/L and 33 U/L for AST, between 4 U/L and 36 U/L for ALT, between 5 U/L and 40 U/L for γGT, between about 61.9 μmol/L and 114.9 μmol/L for men and between 53 μmol/L and 97.2 μmol/L for women for creatinine, less than 125 μg/mL for human subjects below the age of 75 and less than 450 μg/mL for human subjects above the age of 75 for NT-ProBNP, between 50 U/L and 200 U/dL for vWF, less than 52 ng/mL for c-Troponin T, between 4.0 mmol/L and 5.9 mmol/L when fasting for plasma glucose, less than 3.0 mg/L for hsCRP, between 90 mmHg and 139 mmHG for SBP, between 60 mmHg and 89 mmHG for DBP, and between 60-100 bpm for resting heart rate.

The term “gold standard” in the context of clinical trials with human subjects has its regular and common scientific meaning and here refers to a double-blind randomized controlled clinical trial (or double-blind randomized control clinical trial; DBRCCT). A DBRCCT is a type of scientifically sound medical experiment that aims to reduce certain sources of bias when testing the effectiveness of new treatments; this is accomplished by randomly allocating human subjects to two or more treatment/placebo groups, treating the subjects in each separate group differently, and then comparing certain predetermined results and outcomes of the different treatments including treatment with a placebo, with respect to a measured response. At least one group of human subjects—the experimental group—has the intervention being assessed, while at least one other group of human subjects—usually called the control group (placebo or no treatment)—has an alternative condition, such as a placebo or no intervention. The groups are followed under conditions of the trial design to see how effective the experimental intervention was. Treatment efficacy is assessed in comparison to the control. There may be more than one treatment group or more than one control group. The trial may be single-blind, in which the human subjects included in the randomized controlled clinical trial do not know which group they have been assigned to, or may be double-blind, where neither the human subjects nor the individuals administering the treatments have this information. This is accomplished by performing exactly the same procedures, to the extent possible, on all human subjects. Any differences must be intrinsic to the treatments being compared (e.g., administering a test compound or composition versus administering a placebo counterpart). The randomness in the assignment of human subjects to groups reduces selection bias and allocation bias, balancing both known and unknown prognostic factors, in the assignment of treatments. Blinding reduces other forms of experimenter and subject biases. The double-blind RCCT is considered the gold standard for clinical trials. It is commonly used to test the efficacy of medical interventions and non-medical interventions (e.g. testing life-style interventions, testing effects of food supplements) and additionally provides information about adverse effects of the tested treatment, such as drug reactions, food supplement reactions.

Rhodospirillum is a genus in the family Rhodospirillaceae, a family of purple non-sulphur bacteria of the Order Rhodospirillales and the Class Alpha-proteobacteria. Rhodospirillaceae are, among other characteristics, characterized by being phototrophic, and growing both aerobically and anaerobically, using light as an energy source. To that purpose, the bacteria contain chlorophyll b. Within the genus Rhodospirillum, three species are distinguished, e.g. Rhodospirillum rubrum (R. rubrum), Rhodospirillum centenum and Rhodospirillum photometricum. Additionally, four species are not officially recognized, vz. Rhodospirillum salexigens, Rhodospirillum salinarum, Rhodospirillum sodomense, and Rhodospirillum tenue. In the genus Phaeospirillum, another member of the family Rhodospirillaceae, two species are included: Phaeospirillum fulvum and Phaeospirillum molischianum. A preparation of bacterial cells for use according to the invention may very well consist of one species from the genus Rhodospirillum, but mixtures of different Rhodospirillum spp. such as Rhodospirillum rubrum, Rhodospirillum centenum, Rhodospirillum tenue, Rhodospirillum photometricum, Rhodospirillum salexigens, Rhodospirillum salinarum, and/or Rhodospirillum sodomense, or mixtures of Phaeospirillum spp., such as Phaeospirillum fulvum and Phaeospirillum molischianum may also be used. Combinations of Rhodospirillum spp. and Phaeospirillum spp. are also encompassed in the present invention. Preferably, a preparation of Rhodospirillum spp. and/or Phaeospirillum spp. comprises Rhodospirillum rubrum and/or Phaeospirillum molischianum, still more preferably the type species Rhodospirillum rubrum strain ATCC 11170 (strain DSM 467) or strain ATCC 25903 and/or Phaeospirillum molischianum strain DSM 120. (ATCC, American Type Culture Collection; DSMZ, Deutsche Sammlung von Mikroorganismen und Zellkulturen).

The ATCC 25903 strain of Rhodospirillum rubrum has been tested by the European Space Agency as a potential food source in animals. They argue that since R. rubrum cultures do not produce toxins, they can be considered as an edible biomass that could eventually be used as a complementary food source.

### Abbreviations Used

ALT/ALAT, alanine transaminase; AST/ASAT, aspartate transaminase; BMI, Body Mass Index; BP, Blood pressure; bpm, beats per minute; CCMO, Central Committee on Research Involving Human Subjects; CRP, C-reactive protein; CVD, Cardiovascular disease; DBP, Diastolic blood pressure; γGT/GGT, gamma-glutamyltransferase; HDL, High-density lipoprotein cholesterol; h-FABP, Hearty fatty acid binding protein; hsCRP, high sensitivity C-reactive protein; LDL, Low-density lipoprotein cholesterol; METC, Medical research Ethical Committee; In Dutch: Medisch Ethische Toetsing Commissie; MUMC+, Maastricht University Medical Center+; NT-ProBNP, N-terminal prohormone of brain natriuretic peptide; vWF, Von Willebrand factor; POC, Proof of concept study; MRUM, Metabolic Research Unit Maastricht; (S)AE, (Serious) adverse event; SBP, Systolic Blood Pressure; TAG, Triacylglycerol; TC, Total cholesterol; WMO, Medical Research Involving Human Subjects Act; In Dutch: Wet Medisch-wetenschappelijk Onderzoek met Mensen.

### DESCRIPTION OF EMBODIMENTS

The present invention will be described with respect to particular embodiments but the invention is not limited thereto but only by the claims.

It is a goal of the present invention to provide a substance or composition or food supplement or pharmaceutical composition or foodstuff comprising an active ingredient comprising an active (pharmaceutical) ingredient with plasma LDL-cholesterol lowering activity in a subject in need thereof to whom the substance or the composition is administered. Ideally, the substance, etc., does not influence good cholesterol (HDL-cholesterol) levels in the plasma of a human subject in need of LDL-cholesterol plasma level lowering intervention (e.g. since the human subject has a plasma level of LDL-cholesterol of at least 1.8 mM) or in need of LDL-cholesterol plasma level controlling and maintenance intervention (e.g. since the human subject has a plasma level of LDL-cholesterol of less than 3.0 mM, such as less than 2.5 mM, or at most 1.8 mM). Typically, the human subject is 18 years of age or older.

It is an objective of the current invention to provide a compound or a composition comprising said compound, which is one or more of i) safe at a dose suitable for administering to a human subject in need thereof, wherein ‘safe’ refers to an acceptable extent, if occurring at all, of adverse side events in a human subject to whom the compound or composition is administered, such as a (healthy) human subject with a plasma LDL-cholesterol level of 70 mg/dL or higher, ii) active when lowering plasma LDL-cholesterol level in a human subject is considered, and iii) capable of maintaining plasma HDL-cholesterol essentially unaltered or increasing HDL-cholesterol concentration in plasma in an absolute manner or relative manner when compared to plasma LDL-cholesterol level, when the compound or the composition comprising the compound is administered to a human subject in need thereof.

It is additionally an objective of the current invention to provide a compound or composition comprising said compound, which is safe and which has no negative effect on serum values (concentrations) for (health-related) parameters, which are commonly measured to assess vital functioning conditions of the human body such as heart function, liver function and kidney function. These values for health-related parameters are values/concentrations for AST, ALT, γGT, creatinine, NT-ProBNP, vWF, c-troponin T, hsCRP, and plasma glucose levels in the blood serum or plasma of a human subject, and DBP, SBP and resting heart rate.

At least one of the above objectives is achieved by providing R. rubrum cells for use in a method for the lowering of absorption of cholesterol from the intestine and into the circulation of a human subject, preferably 18 years of age or older, such that the level of LDL-cholesterol in the plasma of said human subject is reduced and the level of HDL-cholesterol in the plasma of said human subject remains essentially unaltered, therewith reducing the risk for the occurrence of cardiovascular disease for the human subject subjected to the method. Typically, the provided R. rubrum cells are dried bacterial cells obtained by applying drying such as refractive drying using a refraction dryer known in the art, e.g. at a temperature of 100° C. or less than 100° C. such as 55-100° C., preferably 60-100° C., more preferably 80-100° C., such as 56-90° C., 58-85° C., 60-75° C. and 65-70° C., of cells preferably freshly cultured R. rubrum cells such that (the risk for) damaging of active molecules is limited or even fully prevented (for example to an extent less than the damaging of active molecules typically occurring when R. rubrum cells would be subjected to spray-drying, lyophilisation, freeze-drying, drum-drying, etc.). The R. rubrum cells that are obtained with refractive drying using a refraction dryer known in the art are essentially free of moisture, or may comprise 1% moisture based on the total weight of the dried cells, such that the dry mass of the oven-dried R. rubrum cells after refractive drying is 99-100% based on the weight of the cells. Typically, the R. rubrum cells are provided as a food supplement and/or as a food additive, e.g. as a tablet or capsule ingested shortly before or shortly after or during the human subject consumes a meal, snack, etc., during e.g. breakfast, lunch, diner, in-between-meals snacking, etc. Herewith, one of the objectives is achieved by the provision of a composition or a cell preparation or a functional food ingredient in cholesterol-lowering strategies.

The safety profile of active pharmaceutical ingredients and pharmaceuticals and food ingredients, nutraceuticals, food supplements, food stuffs, animal feed, food and feed products, can be measured by assessment of values or concentrations for several health-related parameters in the blood, plasma and/or serum of a human subject, under influence of such an active pharmaceutical ingredient or pharmaceutical, etc., etc., and such measurements are known to a person skilled in the art. These parameters include, but are not limited to, AST, ALT, γGT, creatinine, NT-ProBNP, vWF, c-troponin T, hsCRP, and plasma glucose levels in the blood of a human subject. Furthermore, DBP, SBP and resting heart rate are selected for assessment before and during the administration of the R. rubrum cells to the human subjects in need thereof (e.g. human subjects having 5.0 mM-8.0 mM total plasma cholesterol). It is beneficial if a therapy or treatment with the R. rubrum cells according to the invention keeps the values or concentrations of the above-mentioned parameters essentially stable and unaltered within the standard normal range in the blood, plasma and/or serum of a treated human subject. It is also beneficial if a therapy or treatment with the R. rubrum cells according to the invention is accompanied with values or concentrations of the above-mentioned parameters that remain stable and essentially unaltered in the blood, plasma and/or serum of a treated human subject.

Thus, it is particularly beneficial when treatment of human subjects and patients in need of lowering their plasma cholesterol levels, e.g. with R. rubrum cells or a pharmaceutical composition of the invention or a food product according to the invention, prevents the increase in serum or plasma levels of any one or more, preferably all of AST, ALT, γGT, creatinine, NT-ProBNP and hsCRP. In addition, it is also beneficial for the health of the human subject to whom R. rubrum cells or a composition comprising R. rubrum cells is administered, that blood serum values for vWf and c-Troponin and plasma glucose concentration remain within the standard normal range during and after treatment with the LDL-cholesterol lowering composition or compound of the invention, i.e. consisting or comprising (dried) R. rubrum cells. Furthermore, it is beneficial for the health of the human subject that the DBP, the SBP and resting heart rate remain within the standard normal range during and after treatment with the LDL-cholesterol lowering composition or compound of the invention, and indeed remain essentially unaltered.

An aspect of the invention relates to Rhodospirillum rubrum cells for use in a method for lowering of LDL-cholesterol concentration in blood of a human subject. An embodiment is the Rhodospirillum rubrum cells for use according to the invention, wherein the values for one or more of AST, ALT, γGT, creatinine, NT-ProBNP and hsCRP essentially remain unaltered in the blood of said human subject. An embodiment is the Rhodospirillum rubrum cells for use according to the invention, wherein the values for one or more of AST, ALT, γGT, creatinine, NT-ProBNP and hsCRP essentially remain within normal or healthy boundaries in the blood of said human subject. An embodiment is the Rhodospirillum rubrum cells for use according to the invention, wherein the values for SBP and/or DBP remain essentially unaltered or remain within normal, healthy boundaries for said subject, when the SBP and the DBP are determined before and during the administration of the cells to said human subject and compared, and/or when the SBP and the DBP are determined at a first day at which the cells are administered to said human subject and at a second day at which the cells are administered to said human subject, and compared. An embodiment is the Rhodospirillum rubrum cells for use according to the invention, wherein the values for resting heart rate remain essentially unaltered or remain within normal, healthy boundaries for said subject, when the resting heart rate is determined before and during the administration of the cells to said human subject and compared, and/or when the resting heart rate is determined at a first day at which the cells are administered to said human subject and at a second day at which the cells are administered to said human subject, and compared.

An aspect of the invention relates to Rhodospirillum rubrum cells for use in the treatment or the prophylaxis of a cardiovascular disease in a human subject. An embodiment is the Rhodospirillum rubrum cells for use according to the invention, wherein the values for one or more of AST, ALT, γGT, creatinine, NT-ProBNP and hsCRP essentially remain unaltered in the blood of said human subject. An embodiment is the Rhodospirillum rubrum cells for use according to the invention, wherein the values for one or more of AST, ALT, γGT, creatinine, NT-ProBNP and hsCRP essentially remain within normal or healthy boundaries in the blood of said human subject. An embodiment is the Rhodospirillum rubrum cells for use according to the invention, wherein the values for SBP and/or DBP remain essentially unaltered or remain within normal, healthy boundaries for said subject, when the SBP and the DBP are determined before and during the administration of the cells to said human subject and compared, and/or when the SBP and the DBP are determined at a first day at which the cells are administered to said human subject and at a second day at which the cells are administered to said human subject, and compared. An embodiment is the Rhodospirillum rubrum cells for use according to the invention, wherein the values for resting heart rate remain essentially unaltered or remain within normal, healthy boundaries for said subject, when the resting heart rate is determined before and during the administration of the cells to said human subject and compared, and/or when the resting heart rate is determined at a first day at which the cells are administered to said human subject and at a second day at which the cells are administered to said human subject, and compared.

An aspect of the invention relates to Rhodospirillum rubrum cells for use in the treatment or prophylaxis of atherosclerosis, dyslipidemia, arteriosclerosis, hypercholesterolemia, familial hypercholesterolemia, hyperlipidemia, an LDL-cholesterol plasma level of at least 70 mg/dL, an LDL-cholesterol plasma level of at least 100 mg/dL, an LDL-cholesterol plasma level of at least 140 mg/dL, an LDL-cholesterol plasma level of at least 200 mg/dL, a total plasma cholesterol level of at least 200 mg/dL, a total plasma cholesterol level of 5.0 mM-8.0 mM, a plasma Lp(a) level of at least 14 mg/dL, ischemia, in a human subject.

An aspect of the invention relates to Rhodospirillum rubrum cells for use in the inhibition of absorption of cholesterol from the intestine of a human subject, such that LDL-cholesterol concentration in the blood of said human subject decreases or is decreased.

An embodiment is the Rhodospirillum rubrum cells for use according to the invention, wherein the HDL-cholesterol concentration in the blood of the human subject remains unaltered or increases or is increased.

An aspect of the invention relates to a non-therapeutic method of lowering LDL-cholesterol concentration in blood of a human subject, the method comprising administering to the human subject a composition comprising of or consisting of Rhodospirillum rubrum cells.

An aspect of the invention relates to a non-therapeutic method of treating or preventing a cardiovascular disease in a human subject, the method comprising administering to the human subject a composition comprising of or consisting of Rhodospirillum rubrum cells.

An aspect of the invention relates to a non-therapeutic method of treating or preventing atherosclerosis, dyslipidemia, arteriosclerosis, hypercholesterolemia, familial hypercholesterolemia, hyperlipidemia, homozygous sitosterolemia, an LDL-cholesterol plasma level of at least 70 mg/dL, an LDL-cholesterol plasma level of at least 100 mg/dL, an LDL-cholesterol plasma level of at least 140 mg/dL, an LDL-cholesterol plasma level of at least 200 mg/dL, a total plasma cholesterol level of at least 200 mg/dL, a total plasma cholesterol level of 5.0 mM-8.0 mM, a plasma Lp(a) level of at least 14 mg/dL, ischemia, in a human subject, the method comprising administering to the human subject a composition comprising of or consisting of Rhodospirillum rubrum cells.

An embodiment is the non-therapeutic method according to the invention, wherein the administration to the human subject of the composition comprising of or consisting of Rhodospirillum rubrum cells inhibits absorption of cholesterol from the intestine of the human subject, such that LDL-cholesterol concentration in the blood of said human subject decreases or is decreased, and preferably such that HDL-cholesterol concentration in the blood of the human subject remains unaltered or increases or is increased.

An aspect of the invention relates to a non-therapeutic method of lowering LDL-cholesterol concentration in blood of a human subject, the method comprising orally administering to the human subject a composition comprising of or consisting of Rhodospirillum rubrum cells

An embodiment is the Rhodospirillum rubrum cells for use according to the invention or the non-therapeutic method of the invention, wherein the concentration of at least one of ALT, AST, γGT, creatinine, NT-ProBNP, vWF, c-Troponin T, plasma glucose and hsCRP remains within a standard normal range and/or remains essentially constant in the serum and/or plasma of the human subject upon administration of the Rhodospirillum rubrum cells to said human subject, preferably all the concentrations remain essentially constant and/or remain within the standard normal range, preferably compared to the concentration(s) determined for said at least one of ALT, AST, γGT, creatinine, NT-ProBNP, vWF, c-Troponin T, plasma glucose and hsCRP before the start of the first administration of the Rhodospirillum rubrum cells and/or during the subsequent further administration(s) of the Rhodospirillum rubrum cells.

An embodiment is the Rhodospirillum rubrum cells for use according to the invention or the non-therapeutic method of the invention, wherein diastolic blood pressure and/or systolic blood pressure of the human subject remain(s) within a standard normal range and/or remain(s) essentially constant upon administration of the Rhodospirillum rubrum cells to said human subject, preferably compared to said blood pressure(s) determined before the start of the first administration of the Rhodospirillum rubrum cells and/or during the subsequent further administration(s) of the Rhodospirillum rubrum cells.

An embodiment is the Rhodospirillum rubrum cells for use according to the invention or the non-therapeutic method of the invention, wherein the resting heart rate of the human subject remains within a standard normal range and/or remains essentially constant upon administration of the Rhodospirillum rubrum cells to said human subject, preferably compared to said resting heart rate determined before the start of the first administration of the Rhodospirillum rubrum cells and/or during the subsequent further administration(s) of the Rhodospirillum rubrum cells.

An embodiment is the Rhodospirillum rubrum cells for use according to the invention, or the non-therapeutic method of the invention, wherein the HDL-cholesterol concentration in the blood of the human subject remains unaltered or increases or is increased during the course of the administration of the Rhodospirillum rubrum cells, preferably once or twice daily administration, preferably compared to the HDL-cholesterol concentration determined before the start of the first administration of the Rhodospirillum rubrum cells and/or during the subsequent further administration(s) of the Rhodospirillum rubrum cells.

An embodiment is the Rhodospirillum rubrum cells for use according to the invention, or the non-therapeutic method of the invention, wherein the human subject has a total plasma cholesterol level of 1.5 mM-16.0 mM, preferably 2.0 mM-12.0 mM, more preferably 3.0 mM-10.0 mM, most preferably 5.0 mM-8.0 mM, before and/or at the start of the first administration and/or during the subsequent further administration(s) of the Rhodospirillum rubrum cells to said human subject.

An embodiment is the Rhodospirillum rubrum cells for use according to the invention, or the non-therapeutic method of the invention, wherein the Rhodospirillum rubrum cells are administered orally to the human subject.

An embodiment is the Rhodospirillum rubrum cells for use according to the invention, or the non-therapeutic method of the invention, wherein the Rhodospirillum rubrum cells are administered to a healthy human subject, preferably a healthy human subject with a total cholesterol concentration in the blood before the first administration of Rhodospirillum rubrum cells and/or during the subsequent further administration(s) of the Rhodospirillum rubrum cells to said human subject, of at least 2.0 mM, such as 3.0 mM-14.0 mM or 5.0 mM-8.0 mM.

An embodiment is the Rhodospirillum rubrum cells for use according to the invention, or the non-therapeutic method of the invention, wherein administering the Rhodospirillum rubrum cells to the human subject results in a decrease of the LDL-cholesterol concentration in the plasma of said human subject, preferably with at least 1%, preferably at least 3%, more preferably at least 5%, more preferably at least 8%, most preferably at least 20%, based on the plasma LDL-cholesterol concentration prior to the first administration of the Rhodospirillum rubrum cells to said human subject, and wherein optionally the plasma HDL-cholesterol concentration remains essentially unaltered or decreases to a smaller extent than the decrease in the plasma LDL-cholesterol concentration, or wherein the plasma HDL-cholesterol concentration increases, preferably the plasma HDL-cholesterol concentration remains essentially unaltered or increases, based on the plasma HDL-cholesterol concentration prior to the first administration of the Rhodospirillum rubrum cells to said human subject.

An embodiment is the Rhodospirillum rubrum cells for use according to the invention, or the non-therapeutic method of the invention, wherein the Rhodospirillum rubrum cells are formulated as a granulate of dried Rhodospirillum rubrum cells obtained by subjecting the cells, preferably freshly cultured cells, to drying, preferably refractive drying at 55° C.-100° C., preferably at 60° C.-100° C., more preferably at 80° C.-100° C., such as 56-98° C., 57-90° C., 58-85° C., 59-80° C., 60-75° C. and 61-69° C., preferably a granulate provided in a capsule such as a gelatin capsule or provided in a sachet.

An embodiment is the Rhodospirillum rubrum cells for use according to the invention, or the non-therapeutic method of the invention, wherein a daily dose of Rhodospirillum rubrum cells to be administered to a human subject is at least 1.0 g, such as at least 2.0 g or 3.0 g, preferably 1.0-100 g, more preferably 2.0-50 g, most preferably 4.0-20 g, such as 5.0 gram.

Embodiments are the Rhodospirillum rubrum cells for use according to the invention or the non-therapeutic method according to the invention, wherein the Rhodospirillum rubrum cells are administered to a human subject having an LDL-cholesterol level in plasma of at least 1.8 mmol/L (70 mg/dL), or at least 2.59 mmol/L (100 mg/dL), or at least 3.34 mmol/L (129 mg/dL), or at least 4.0 mmol/L, such as at least 5.2 mmol/L (200 mg/dL) or between 5.0 mM and 8.0 mM.

An embodiment is the Rhodospirillum rubrum cells for use according to the invention or the non-therapeutic method according the invention, wherein Rhodospirillum rubrum cells are administered to a human subject to whom at least one active pharmaceutical ingredient is (co-)administered, such as at least one active pharmaceutical ingredient selected from a statin, niacin, fenofibrate, ezetimibe, colesevelam, mipomersen, lomitapide, a PCSK9 inhibitor, alirocumab, evolocumab, ETC-1002, a CETP inhibitor, anacetrapib, evacetrapib, WAY-252623, a blood-pressure lowering compound, hydrochlorothiazide, preferably a statin such as atorvastatin, fluvastatin, pravastatin, rosuvastatin, simvastatin, lovastatin and/or ezetimibe.

An embodiment is the Rhodospirillum rubrum cells for use according to the invention or the non-therapeutic method according the invention, wherein the statin and/or the ezetimibe are administered to the subject at a lower total daily dose when co-administered with Rhodospirillum rubrum cells, compared to the daily dose during standard therapeutic treatment with the statin or with the ezetimibe.

An embodiment is the Rhodospirillum rubrum cells for use according to the invention or the non-therapeutic method according the invention, wherein the Rhodospirillum rubrum cells are administered orally to the human subject.

An embodiment is the Rhodospirillum rubrum cells for use according to the invention or the non-therapeutic method according the invention, wherein the Rhodospirillum rubrum cells are administered to a healthy human subject, preferably a healthy human subject with a total cholesterol concentration in the blood before the first administration of Rhodospirillum rubrum cells, of at least 5.0 mM, such as 5.0 mM-8.0 mM.

An embodiment is the Rhodospirillum rubrum cells for use according to the invention or the non-therapeutic method according the invention, wherein administering the Rhodospirillum rubrum cells to a human subject results in lowering of the plasma LDL-cholesterol concentration, preferably to a plasma concentration of less than 3.34 mmol/L, preferably less than 2.59 mmol/L, more preferably to a plasma LDL-cholesterol concentration of less than 1.8 mmol/L.

An embodiment is the Rhodospirillum rubrum cells for use according to the invention or the non-therapeutic method according the invention, wherein administering the Rhodospirillum rubrum cells to a human subject results in a decrease of the LDL-cholesterol concentration in the plasma of said human subject, wherein the plasma HDL-cholesterol concentration remains essentially unaltered or decreases to a smaller extent than the decrease in the plasma LDL-cholesterol concentration, or wherein the plasma HDL-cholesterol concentration increases, preferably the plasma HDL-cholesterol concentration remains essentially unaltered or increases.

An embodiment is the Rhodospirillum rubrum cells for use according to the invention or the non-therapeutic method according the invention, wherein administering the Rhodospirillum rubrum cells to a human subject results in a decrease of the plasma LDL-cholesterol concentration in said subject with at least 1%, preferably at least 5%, more preferably at least 6%, more preferably at least 10%, most preferably at least 20%, based on the plasma LDL-cholesterol concentration prior to the administration of the Rhodospirillum rubrum cells to said human subject.

An embodiment is the Rhodospirillum rubrum cells for use according to the invention or the non-therapeutic method according the invention, wherein administering the Rhodospirillum rubrum cells to a subject results in a decrease of the plasma LDL-cholesterol concentration to, or maintenance of the plasma LDL-cholesterol concentration at, a plasma LDL-cholesterol concentration of less than 200 mg/dL, or less than 159 mg/dL, or less than 129 mg/dL, preferably less than 100 mg/dL, such as at or less than 70 mg/dL.

An embodiment is the Rhodospirillum rubrum cells for use according to the invention or the non-therapeutic method according the invention, wherein the Rhodospirillum rubrum cells are formulated as an oral dosage form, preferably a solid oral dosage form or a liquid oral dosage form, preferably a liquid oral dosage form comprising an oil or a drink.

An embodiment is the Rhodospirillum rubrum cells for use according to the invention or the non-therapeutic method according the invention, wherein the Rhodospirillum rubrum cells are formulated as a granulate, preferably a granulate provided in a capsule such as a gelatin capsule or in a sachet.

An embodiment is the Rhodospirillum rubrum cells for use according to the invention or the non-therapeutic method according the invention, wherein the Rhodospirillum rubrum cells are dried Rhodospirillum rubrum cell granules obtained by subjecting freshly cultured cells to refractive drying at 55° C.-100° C., preferably at 60° C.-100° C., more preferably at 80° C.-100° C., such as 57-67° C. and 58-62° C.

An embodiment is the Rhodospirillum rubrum cells for use according to the invention or the non-therapeutic method according the invention, wherein a daily dose of Rhodospirillum rubrum cells to be administered to a human subject is 100 mg-50 g per day, preferably 200 mg-25 g per day, more preferably 500 mg-10 g per day, most preferably 1 g-5 g per day, such as 0.25 g, 0.50 g, 1.0 g, 2.0 g, 3.0 g, 4.0 g, 5.0 g, 7.5 g, 15 g, 20 g, 30 g, or 40 g.

An embodiment is the Rhodospirillum rubrum cells for use according to the invention or the non-therapeutic method according the invention, wherein a daily dose of Rhodospirillum rubrum cells to be administered to a human subject is at least 1.0 g, such as at least 5.0 g, preferably 1.0-100 g, more preferably 1.0-50 g, most preferably 5.0-20 g.

An embodiment is the Rhodospirillum rubrum cells for use according to the invention or the non-therapeutic method according the invention, wherein the Rhodospirillum rubrum cells are dosed to provide an amount of between 0.1 g and 50 g of Rhodospirillum rubrum cells per day.

An embodiment is the Rhodospirillum rubrum cells for use according to the invention or the non-therapeutic method according the invention, wherein a daily dose of Rhodospirillum rubrum cells to be administered to a human subject are two half-daily doses each comprising half of the daily dose, wherein preferably a first half-daily dose is administered 1-2 hours before lunch, 1-2 hours after lunch or during lunch taken by the human subject, and a second half-daily dose is administered 1-2 hours before dinner, 1-2 hours after dinner or during dinner taken by the human subject, preferably during lunch and during dinner.

An embodiment is the Rhodospirillum rubrum cells for use according to the invention or the non-therapeutic method according the invention, wherein administering the Rhodospirillum rubrum cells to a subject maintains the LDL-cholesterol concentration in the plasma of the subject at a level of less than 159 mg/dL, preferably less than 129 mg/dL, more preferably less than 100 mg/dL, most preferably at or less than 70 mg/dL, such as between 50 mg/dL and 159 mg/dL.

Beneficially to the objectives of the inventors, R. rubrum cultures do not produce toxins. Therefore, the inventors consider R. rubrum cells and compositions comprising R. rubrum cells or a fraction, extract, compounds derived therefrom, as an edible biomass that is suitable for use as a complementary food source for human subjects (and in addition also for animals such as pigs, poultry such as chickens, broilers, laying hen, mammals, vertebrates, carnivores, domestic animals, farmed animals, dogs, cats, fish).

Plasma cholesterol is the single cardiovascular risk factor. Drugs or food supplements that target this risk factor would therefore be successful, and agents commonly known as statins that inhibit 3-hydroxy-3-methylglutaryl coenzyme A reductase, therewith blocking the rate-limiting step in cholesterol synthesis, are the most widely prescribed agents. Despite evidence that the use of these agents, in almost any setting for cardiovascular risk factor management, can reduce morbidity and mortality, compliance and goal attainment remain below acceptable standards. A reason for this is the need to use higher or more potent doses of statins, accompanied with the increased dose-related side-effect profiles, e.g. myalgia, myositis, drug-drug interactions.

Resins, such as cholestyramine and colesevalam, bind bile acids and prevent absorption of intestinal cholesterol by preventing its solubilization into micelles. In addition, these compounds cause loss of bile acids, each molecule of bile acid having been generated from a molecule of cholesterol by the liver, and thus cause a drain on the body pools of cholesterol. The use of these compounds is limited, as these compounds can cause considerable gastro-enterologic distress, as well as may reduce the bioavailability of any other drug that is co-administered at the same time.

Natural fiber can also lower cholesterol concentration in blood, in part by increasing the loss of bile acids in the stool. The fortification of margarines with plant sterols or their metabolites that compete with cholesterol for solubilization into micelles have been available for lowering plasma cholesterol levels. In general, all of these agents that operate to lower cholesterol absorption or to induce increased fecal loss, are moderately effective and need to be used in gram doses that act via a mass-action effect. In contrast, the inventors now established that already a dose-response is achieved when lowering LDL-cholesterol concentration in blood of human subjects is considered, when as low amounts of R. rubrum cells as 0.25 g/daily-1.0 g daily are administered to the human subjects, half of the dose during dinner time, half of the dose during lunch time.

In a mouse model of atherosclerosis, i.e. the apolipoprotein (Apo) E knockout mouse, the administration of ezetimibe led to a significant reduction of lipid-laden plaques in the aortas of these animals compared with controls. Thus, blocking dietary cholesterol in this animal model had significant benefits on atherosclerotic plaque formation. Therefore, it is expected that also R. rubrum cell monotherapy has an effect on lipid-laden plaques, i.e. reducing such plaques, by inhibiting absorption of dietary cholesterol together with inhibiting absorption of biliary cholesterol in the intestine of treated human subjects.

The inventors established that monotherapy with R. rubrum cells is effective in lowering plasma low-density lipoprotein (LDL) cholesterol by at least about 2-6% and results in maintenance of the HDL-cholesterol concentration in the blood of treated human subjects.

The average percentage of cholesterol absorbed by normal healthy individuals is about 55%, with a Gaussian distribution about this mean. Therefore, it is expected that treatment of human subjects with R. rubrum cells will be improvingly effective when the treatment is in combination with statin drugs. For comparison, in placebo-controlled studies, the combination of 10 mg of ezetimibe with a starting dose of a statin resulted in an LDL-cholesterol lowering in blood that was the equivalent of using the maximum dose of the statin alone. This combination appeared to result in a statin-sparing effect, and it was hypothesized that the basis for this synergistic effect is likely to be a greater clearance of LDL-cholesterol particles by the liver. This synergistic effect of treatment of a human subject with a combination of ezetimibe and a statin is seen with all of the statins tested (simvastatin, atorvastatin, lovastatin pravastatin) and in general resulted in a further 21% to 23% LDL-cholesterol lowering in the blood. R. rubrum cells monotherapy has now already been proven by the inventors as being an effective LDL-cholesterol lowering composition, by inhibiting cholesterol absorption in the intestine. Likely, combination therapy by combining R. rubrum cells with e.g. a statin at relatively low dose and/or ezetimibe, will result in even further lowering of the LDL-cholesterol blood levels.

With the combination of R. rubrum cells with a fibrate, similar to the combination of ezetimibe and a fibrate, synergism is expected, similar to the combination of R. rubrum cells with either a statin at sub-optimal dose when the statin is used without R. rubrum, or ezetimibe, or both a statin and ezetimibe, because R. rubrum cells and a statin target separate pathways (peroxisome proliferator-activated receptor-a-mediated gene expression changes for fibrates versus intestinal cholesterol absorption for R. rubrum cells, similar to ezetimibe). The inventors established that the active petroleum-ether fraction of R. rubrum (see example 5, here below) consists of about 1.5% dry matter based on the total weight of the dried R. rubrum cells, and therewith, these 1.5% contains the active compound(s) of R. rubrum, which is/are at the basis of the inhibitory effect on cholesterol absorption in the intestine. This 1.5% fraction contains a series of carotenoids that are supposed to underlie the inhibitory effect in the intestine of human subjects.

An aspect of the invention relates to a pharmaceutical composition comprising a pharmaceutically effective amount of Rhodospirillum rubrum cells and optionally a pharmaceutically acceptable excipient, for use in a method for the lowering of LDL-cholesterol in blood plasma of a human subject.

A further aspect of the invention relates to a pharmaceutical composition comprising a pharmaceutically effective amount of Rhodospirillum rubrum cells and optionally a pharmaceutically acceptable excipient, for use in a method for the treatment or prophylaxis of any one or more of cardiovascular disease, atherosclerosis, dyslipidemia, arteriosclerosis, hypercholesterolemia, familial hypercholesterolemia, hyperlipidemia, homozygous sitosterolemia, an LDL-cholesterol plasma level of at least 70 mg/dL, an LDL-cholesterol plasma level of at least 100 mg/dL, an LDL-cholesterol plasma level of at least 140 mg/dL, an LDL-cholesterol plasma level of at least 200 mg/dL, a total plasma cholesterol level of at least 200 mg/dL, a total plasma concentration in the blood of at least 5.0 mM such as between 5.0 mM and 8.0 mM, an Lp(a) level of at least 14 mg/dL, inflammation, inflammatory disease, ischemia, infection.

An embodiment is the pharmaceutical composition for use according to the invention, wherein the concentration of at least one of ALT, AST, γGT, creatinine, NT-ProBNP, vWF, c-Troponin T, plasma glucose and hsCRP remains within a standard normal range and/or remains essentially constant in the serum and/or plasma of the human subject upon administration of the pharmaceutical composition to said human subject, preferably all the concentrations remain essentially constant and/or remain within the standard normal range, preferably compared to the concentration(s) determined for said at least one of ALT, AST, γGT, creatinine, NT-ProBNP, vWF, c-Troponin T, plasma glucose and hsCRP before the start of the first administration of the pharmaceutical composition and/or during the subsequent one or more further administrations of the pharmaceutical composition.

An embodiment is the pharmaceutical composition for use according to the invention, wherein diastolic blood pressure and/or systolic blood pressure of the human subject remain(s) within a standard normal range and/or remain(s) essentially constant upon administration of the pharmaceutical composition to said human subject, preferably compared to said blood pressure(s) determined before the start of the first administration of the pharmaceutical composition and/or during the subsequent further administration(s) of the pharmaceutical composition.

An embodiment is pharmaceutical composition for use according to the invention, wherein the resting heart rate of the human subject remains within a standard normal range and/or remains essentially constant upon administration of the Rhodospirillum rubrum cells to said human subject, preferably compared to said resting heart rate determined before the start of the first administration of the Rhodospirillum rubrum cells and/or during the subsequent further administration(s) of the Rhodospirillum rubrum cells.

An embodiment is the pharmaceutical composition for use according to the invention, wherein the human subject has a total plasma cholesterol level of 1.5 mM-16.0 mM, preferably 2.0 mM-12.0 mM, more preferably 3.0 mM-10.0 mM, most preferably 5.0 mM-8.0 mM, before and/or at the start of the first administration and/or during the subsequent administration(s) of the pharmaceutical composition to said human subject.

An embodiment is the pharmaceutical composition for use according to the invention, wherein the Rhodospirillum rubrum cells are the sole active pharmaceutical ingredient in said pharmaceutical composition.

An embodiment is the pharmaceutical composition for use according to the invention, wherein the Rhodospirillum rubrum cells are administered to the human subject as replacement therapy such as therapy replacing a statin and/or ezetimibe, or are administered to the human subject in combination with a lower dose of (a) LDL-cholesterol lowering pharmaceutical compound(s) administered to the human subject than the dose of such (a) LDL-cholesterol lowering pharmaceutical compound(s) administered to the human subject before administration of the pharmaceutical composition started.

An embodiment is the pharmaceutical composition for use according to the invention, wherein the Rhodospirillum rubrum cells in the pharmaceutical composition are the Rhodospirillum rubrum cells of any one of the previous embodiments.

An embodiment is the pharmaceutical composition for use according to the invention, wherein a daily dose of the pharmaceutical composition contains at least 1.0 g Rhodospirillum rubrum cells, such as at least 2.0 g or at least 3.0 g, preferably 1.0-100 g, more preferably 2.0-50 g, most preferably 4.0-20 g such as 5.0-10.0 gram.

An embodiment is the pharmaceutical composition for use according to the invention, wherein the Rhodospirillum rubrum cells are the sole active pharmaceutical ingredient in said pharmaceutical composition.

An embodiment is the pharmaceutical composition for use according to the invention, wherein the pharmaceutical composition is administered to a subject to whom at least one further active pharmaceutical ingredient is administered, such as an active pharmaceutical ingredient selected from a statin, niacin, fenofibrate, ezetimibe, colesevelam, mipomersen, lomitapide, a PCSK9 inhibitor, alirocumab, evolocumab, ETC-1002, a CETP inhibitor, anacetrapib, evacetrapib, WAY-252623, a blood-pressure lowering compound, hydrochlorothiazide, or any combination thereof such as a statin and a PCSK9 inhibitor or a statin and ezetimibe, preferably a statin such as atorvastatin, fluvastatin, pravastatin, rosuvastatin, simvastatin, lovastatin, and/or ezetimibe.

An embodiment is the pharmaceutical composition for use according to the invention, wherein the Rhodospirillum rubrum cells are administered to the human subject as replacement therapy such as therapy replacing a statin and/or ezetimibe, or are administered to the human subject in combination with a lower dose of (a) LDL-cholesterol lowering pharmaceutical compound(s) administered to the human subject than the dose of such (a) LDL-cholesterol lowering pharmaceutical compound(s) administered to the human subject before administration of Rhodospirillum rubrum cells started.

An embodiment is the pharmaceutical composition for use according to the invention, wherein the pharmaceutical composition is administered to a human subject suffering from an LDL-cholesterol plasma level of higher than 70 mg/dL, an LDL-cholesterol plasma level of at least 100 mg/dL, an LDL-cholesterol plasma level of at least 140 mg/dL, an LDL-cholesterol plasma level of at least 200 mg/dL, a total cholesterol concentration in the blood of at least 5.0 mM such as 5.0 mM-8.0 mM, a cardiovascular disease, hypercholesterolemia, atherosclerosis.

An embodiment is the pharmaceutical composition for use according to the invention, wherein the Rhodospirillum rubrum cells are the Rhodospirillum rubrum cells of any one of the previous embodiments of the invention.

Cholesterol synthesis in the liver is highly sensitive to the amount of (dietary) cholesterol that reaches the liver from the intestine via the chylomicron-remnant pathway. The Western-type human diet provides approximately 400 mg of cholesterol per day. On top of this, the liver secretes approximately 1 gram of cholesterol into bile per day. Intestinal cholesterol absorption efficiency in humans is highly variable, ranging from 15% to 85% in healthy subjects. After uptake by enterocytes, cholesterol is packed with triglycerides into chylomicrons and secreted into the lymph. In the circulation, the triglycerides are rapidly hydrolyzed and free fatty acids are taken up by the peripheral tissues. Cholesterol-enriched chylomicron remnants are subsequently cleared by the liver. Since chylomicron remnants, which contain most of the cholesterol that is being absorbed from the intestine, are rapidly taken up by the liver, interference with the absorption process directly influences hepatic cholesterol synthesis.

The inventors now established that lowering the LDL-cholesterol level in the blood, e.g. the plasma/serum of a human subject can be the consequence of inhibition of the absorption of cholesterol and/or cholesterol esters from the gastrointestinal tract. A portion of cholesterol in the body and in the circulation originates from dietary intake, and in addition a portion of the cholesterol present in a subject originates from de novo synthesis in amongst others and predominantly the liver. Cholesterol biosynthesized in the liver is in part transported and stored in the gall bladder, in the bile. The bile with the cholesterol is excreted to the gastrointestinal tract when required, i.e. when the subject consumes fat-rich food products, etc. It is known that a large portion of bile cholesterol is excreted with the feces, with the remainder of the bile cholesterol being taken up from the intestine together with a portion of dietary cholesterol. Amongst other lipophilic compounds, tri-acyl glycerides and cholesterol from the food intake, together with the cholesterol transferred from the gall bladder to the intestine with the bile, are solubilized first as emulsion particles and then in bile salt micelles. Subsequently, intestinal mucosal cells (enterocytes) absorb the mixed bile salt micelles comprising the cholesterol. Then, the cholesterol is transferred into the lymphatic circulation as part of chylomicrons and also as part of very low density lipoproteins (VLDLs), which are transferred to the blood circulation. Relocation and transfer of cholesterol comprising particles is based on passive simple diffusion and/or may be based on scavenger receptor mediated transfer, for example with involvement of scavenger receptor B-I and/or CD36, although involvement of these receptors is debated. In the bloodstream the cholesterol-loaded chylomicrons transfer (in part) into chylomicron remnants carrying cholesterol, which are taken up from the circulation by the liver. The VLDL-cholesterol particles are degraded into intermediate density lipoproteins (IDLs) comprising the cholesterol, and further into low-density lipoprotein-cholesterol upon discarding tri-acyl glycerol molecules from the IDL particles. The LDL-cholesterol particles are bound by LDL-receptor molecules on the surface of organ cells such as the liver. Upon binding, the LDL-cholesterol is taken up by these LDL-receptor carrying cells.

Cholesterol taken up by liver cells is in part relocated to the gall bladder and becomes part of the stored bile, to be secreted into the intestine after food intake. Cholesterol taken up by liver cells is also in part transferred to the endoplasmic reticulum during assembly of VLDL particles comprising cholesterol. The cholesterol in the VLDL-cholesterol particles originate in part from de novo cholesterol synthesis by the liver cells and in part from dietary cholesterol if present in the diet of the subject. The liver cells excrete the VLDL-cholesterol into the blood circulation. In the blood circulation, the VLDL-cholesterol are transformed into IDL-cholesterol, which (partly) further transforms into LDL-cholesterol.

The inventors now established that the LDL-lowering effect of R. rubrum cells is based on the following principle of inhibition of cholesterol uptake from the diet from the intestine into the circulation. In the intestine, (compounds in) the R. rubrum cells compete with cholesterol during the process of formation of the lipid emulsion, such that uptake of cholesterol by the lipid emulsion is inhibited at the expense of increased uptake of (compounds in) the R. rubrum cells by the lipid emulsion. As a consequence, less cholesterol, which originates from the bile and, if part of the diet, from food, etc., is transferrable to bile salt micelles, required for ultimate release into the lymph system and blood circulation. As a consequence, the hepatocytes take up more circulating LDL-cholesterol, therewith contributing to lowering of the plasma level of LDL-cholesterol. In addition, and without wishing to be bound by any theory, in contrast, compounds derived from the R. rubrum cells are delivered to the circulation as part of the chylomicrons and VLDL particles. The liver takes up the chylomicron remnants comprising these compounds, originating from the chylomicrons, and therewith the compounds become part of the pool of fat soluble compounds present and stored in liver cells, together with de novo synthesized cholesterol by the liver cells and cholesterol taken up by liver cells. In liver cells assembly of VLDL-cholesterol particles occurs. Since the compounds are also present in the pool of fat-soluble compounds to be incorporated in VLDL, the compound(s) again can compete with the cholesterol for incorporation in the VLDL particle. Less cholesterol is secreted into the blood circulation with the VLDL, as a consequence. Further, also less IDL-cholesterol can be formed from VLDL, and then also less LDL-cholesterol. Indeed, compounds present in R. rubrum cells such as polar carotenoids are determined in the blood plasma of human subjects, as part of LDL-particles. Furthermore, a lower chylomicron-cholesterol level in the blood due to inhibition of cholesterol uptake from the intestine by the presence of polar carotenoids in the intestine, induces upregulation of LDL-receptor on hepatocytes, which as a result increases uptake of circulating LDL-cholesterol by the liver cells. Increased uptake of LDL-cholesterol by the hepatocytes additionally contributes to a lower level of blood LDL-cholesterol. The mode of action at least in part resembles the mode of action of ezetimibe, for the cholesterol-absorption inhibitory activity. In addition, or alternatively, as part of the inhibitory activity of R. rubrum cells and as such of compound(s) present therein, when lowering plasma level of LDL-cholesterol is considered, also (or solely) inhibition of enzymes and proteins involved in cholesterol synthesis and transport such as Niemann-Pick C1 Like 1, ATP-binding cassette transporters such as ABCG5 and ABCG8, and 3-hydroxy-3-methylglutaryl-CoA reductase, by the compounds present in R. rubrum cells may occur, and in particular interference in Niemann-Pick C1 Like 1 biology may occur, resulting in lowering of cholesterol uptake from the intestine and therewith lowering of plasma levels of LDL-cholesterol, leaving plasma levels of HDL-cholesterol essentially unaltered.

Identification of the Niemann-Pick C1 like 1 (NPC1L1) protein as a crucial molecule involved in cholesterol uptake by enterocytes and identification of Abcg5 and Abcg8 proteins as (intestinal) cholesterol efflux transporters, has provided definite proof that cholesterol absorption is a protein-mediated, selective and active process. The identification of NPC1L1 has been facilitated by the discovery of cholesterol absorption inhibitor “ezetimibe” (IUPAC name: (3R,4S)-1-(4-fluorophenyl)-3-[(3S)-3-(4-fluorophenyl)-3-hydroxypropyl]-4-(4-hydroxyphenyl)azetidin-2-one). Ezetimibe is a sterol absorption inhibitor that reduces diet-induced hypercholesterolemia in humans. The NPC1L1 protein is a cholesterol transporter in intestinal cells. NPC1L1 is expressed in the intestine at the brush border membrane and Npc1l1-deficient mice show a 69% reduction in fractional cholesterol absorption. The compound ezetimibe glucuronide, the active molecule, binds to cells expressing NPC1L1.

The R. rubrum cells of the invention are suitably co-administered with hydroxy-substituted azetidinones which are proven to be useful as hypocholesterolemic agents in the treatment and prevention of atherosclerosis, and with a combination of such a hydroxy-substituted azetidinone and a cholesterol biosynthesis inhibitor for the treatment and prevention of atherosclerosis. Typically, the hydroxy-substituted azetidinone is ezetimibe and typically the cholesterol biosynthesis inhibitor is a statin. Equally preferred are the R. rubrum cells of the invention that are applied in a therapeutic treatment regimen or in a dietary intervention for lowering LDL-cholesterol in blood, wherein the R. rubrum cells partly or completely replace previous treatment with a statin and/or previous treatment with ezetimibe.

An aspect of the invention relates to a pharmaceutical composition for the treatment or prevention of atherosclerosis, or for the reduction of plasma cholesterol levels, comprising an effective amount of R. rubrum cells, alone or in combination with a cholesterol biosynthesis inhibitor and/or in combination with ezetimibe, in a pharmaceutically acceptable carrier.

An aspect of the invention relates to use of R. rubrum cells for the manufacture of a medicament for the treatment or prevention of atherosclerosis, or for the reduction of plasma cholesterol levels, in a human subject.

An aspect of the invention relates to a process for the preparation of a pharmaceutical composition of the invention which comprises admixing R. rubrum cells as defined in any of the embodiments of the invention with a pharmaceutically acceptable carrier.

An embodiment is the process for preparing a pharmaceutical composition of the invention comprising admixing the R. rubrum cells of the invention with a cholesterol biosynthesis inhibitor and/or with ezetimibe, and with a pharmaceutically acceptable carrier.

An aspect of the inventions relates to the use of R. rubrum cells according to any of the embodiments for the manufacture of a medicament for the combined use with a cholesterol biosynthesis inhibitor and/or ezetimibe in the treatment or prevention of atherosclerosis, or for the reduction of plasma cholesterol levels, preferably LDL-cholesterol level.

An aspect of the invention relates to the use of a cholesterol biosynthesis inhibitor for the manufacture of a medicament for the combined use with R. rubrum cells according to any of the embodiments of the invention, in the treatment or prevention of atherosclerosis, or for the reduction of plasma cholesterol levels.

An embodiment is the pharmaceutical composition of the invention wherein the cholesterol biosynthesis inhibitor is selected from the group consisting of HMG CoA reductase inhibitors, squalene synthesis inhibitors and squalene epoxidase inhibitors.

An embodiment is the pharmaceutical composition of the invention wherein the cholesterol biosynthesis inhibitor is selected from the group consisting of lovastatin, pravastatin, fluvastatin, simvastatin, CI-981, DMP-565, L-659,699, squalestatin 1 and NB-55 598.

An embodiment is the use of the invention, wherein the cholesterol biosynthesis inhibitor is selected from the group consisting of HMG CoA reductase inhibitors, squalene synthesis inhibitors and squalene epoxidase inhibitors, or is selected from the group consisting of lovastatin, pravastatin, fluvastatin, simvastatin, CI-981, DMP-565, L-659,699, squalestatin 1 and NB-55 598.

An embodiment is the Rhodospirillum rubrum cells for use according to the invention or the non-medical method of the invention, wherein a human subject is administered the Rhodospirillum rubrum cells in combination with any one of:
rel 3(R)-(2(R)-hydroxy-2-phenylethyl)-4(R)-(4-methoxyphenyl)-1-phenyl-2-azetidinone;
rel 3(R)-(2(R)-hydroxy-2-phenylethyl)-4(S)-(4-methoxyphenyl)-1-phenyl-2-azetidinone;
3(S)-(1 (S)-hydroxy-3-phenylpropyl)-4(S)-(4-methoxyphenyl)-1-phenyl-2-azetidinone;
3(S)-(1 (R)-hydroxy-3-phenylpropyl)-4(S)-(4-methoxyphenyl)-1-phenyl-2-azetidinone; 3(R)-(1 (R)-hydroxy-3-phenylpropyl)-4(S)-(4-methoxyphenyl)-1-phenyl-2-azetidinone;
rel-3(R)-[(S)-hydroxy-(2-naphthalenyl)methyl]-4(S)-(4-methoxyphenyl)-1-phenyl-2-azetidinone;
rel-3(R)-[(R)-hydroxy-(2-naphthalenyl)methyl]-4(S)-(4-methoxyphenyl)-1-phenyl-2-azetidinone;
3(R)-(3(R)-hydroxy-3-phenylpropyl)-1,4(S)-bis-(4-methoxyphenyl)-2-azetidinone;
3(R)-(3(S)-hydroxy-3-phenylpropyl)-1,4(S)-bis-(4-methoxyphenyl)-2-azetidinone;
4(S)-(4-hydroxyphenyl)-3(R)-(3(R)-hydroxy-3-phenylpropyl)-1-(4-methoxyphenyl)-2-azetidinone;
4(S)-(4-hydroxyphenyl)-3(R)-(3(S)-hydroxy-3-phenylpropyl)-1-(4-methoxyphenyl)-2-azetidinone;
rel 3(R)-[3(RS)-hydroxy-3-[4-(methoxymethoxy)-phenyl]propyl]-1,4(S)-bis-(4-methoxyphenyl)-2-azetidinone;
1-(4-fluorophenyl)-3(R)-[3(S)-(4-fluorophenyl)-3-hydroxypropyl)]-4(S)-(4-hydroxyphenyl)-2-azetidinone;
1-(4-fluorophenyl)-3(R)-[3(R)-(4-fluorophenyl)-3-hydroxypropyl)]-4(S)-(4-hydroxyphenyl)-2-azetidinone;
4(S)-[4-(acetyloxy)phenyl]-3(R)-(3(R)-hydroxy-3-phenylpropyl)-1-(4-methoxyphenyl)-2-azetidinone;
4(S)-[4-(acetyloxy)phenyl]-3(R)-(3(S)-hydroxy-3-phenylpropyl)-1-(4-methoxyphenyl)-2-azetidinone;
1-(4-fluorophenyl)-3(R)-[3(S)-(4-fluorophenyl))-3-hydroxypropyl)]-4(S)-[4-(phenylmethoxy)phenyl]-2-azetidinone;
3(R)-[3(R)-acetyloxy)-3-phenylpropyl]-1,4(S)-bis-(4-methoxyphenyl)-2-azetidinone;
3(R)-[3(S)-acetyloxy)-3-phenylpropyl]-1,4(S)-bis-(4-methoxyphenyl)-2-azetidinone;
3(R)-[3(R)-(acetyloxy)-3-(4-fluorophenyl)propyl]-4(S)-[4-(acetyloxy)phenyl]-1-(4-fluorophenyl)-2-azetidinone;
3(R)-[3(S)-(acetyloxy)-3-(4-fluorophenyl)propyl]-4(S)-[4-(acetyloxy)phenyl]-1-(4-fluorophenyl)-2-azetidinone;
3(R)-[3(R)-(acetyloxy)-3-(4-chlorophenyl)propyl]-4(S)-[4-(acetyloxy)phenyl]-1-(4-chlorophenyl)-2-azetidi-none;
3(R)-[3(S)-(acetyloxy)-3-(4-chlorophenyl)propyl]-4(S)-[4-(acetyloxy)phenyl]-1-(4-chlorophenyl)-2-azetidinone; and
rel 1-(4-fluorophenyl)-4(S)-(4-hydroxyphenyl)-3(R)-(1 (R)-hydroxy-3-phenylpropyl)-2-azetidinone.

Typically, any one or more of these compounds is combined with the Rhodospirillum rubrum cells for co-administration or sequential administration, such that the daily dose of such a compound is between 1 mg and 50 mg. Typically, when used in a non-medical method or when used in a therapeutic regimen, such compound induces a lowering of the total serum cholesterol concentration of between about 12.5% and 60%. Synergistic effects of treatment of Rhodospirillum rubrum cells together with such a compound, e.g. ezetimibe, are likely to occur.

An aspect of the current invention relates to a food supplement or a feed supplement with LDL-cholesterol lowering properties, comprising R. rubrum cells, preferably dried R. rubrum cells preferably obtained with refractive drying at a temperature of e.g. about 55-100° C., preferably about 60-100° C., more preferably about 80-100° C., such as 56-90° C., 57-85° C., 58-80° C., 59-75° C., 60-70° C., and 61-66° C., using a refraction dryer known in the art.

An aspect of the invention relates to a food supplement with cholesterol-lowering properties when ingested by a human subject, comprising Rhodospirillum rubrum cells, wherein the Rhodospirillum rubrum cells are dried Rhodospirillum rubrum cell granules obtained by subjecting freshly cultured cells to refractive drying at 55° C.-100° C., preferably at 60° C.-100° C., more preferably at 80° C.-100° C. or 55° C.-65° C.

An aspect of the invention relates to a foodstuff comprising a food supplement according to the invention.

Embodiments are the food supplement of the invention or the foodstuff of the invention, wherein the Rhodospirillum rubrum cells are the Rhodospirillum rubrum cells of any of the embodiments of the invention.

An aspect of the invention relates to a food supplement with cholesterol-lowering properties when orally ingested by a human subject, comprising Rhodospirillum rubrum cells, wherein the Rhodospirillum rubrum cells are dried Rhodospirillum rubrum cell granules obtained by subjecting Rhodospirillum rubrum cells, preferably freshly cultured cells, to drying, preferably refractive drying at 55° C.-100° C., preferably at 60° C.-100° C., more preferably at 80° C.-100° C., such as 56-68° C., 57-66° C., 58-64° C., 59-63° C., 60-70° C., and 55-71° C., preferably Rhodospirillum rubrum cell granules provided in a capsule such as a gelatin capsule or in a sachet.

An aspect of the invention relates to a foodstuff comprising a food supplement according to the invention.

An embodiment is the food supplement of the invention or the foodstuff of the invention, wherein the Rhodospirillum rubrum cells are the Rhodospirillum rubrum cells of any one of the previous embodiments.

In the present invention a food supplement and a feed supplement is defined as a formulation that is consumed in addition to a normal diet and that contains compounds or components that do not occur in a normal diet, or that occur in low amounts or in insufficient amounts, while sufficient or increased consumption of these components is desired. Preferably, a food supplement according to the invention is composed such that it is suitable for human consumption. Consequently, a food supplement as defined in the present invention should preferably have a texture, taste and smell, but also a nutritional value, that makes the supplement suitable for human consumption.

Preferably, a feed supplement according to the invention is composed such that it is suitable for animal consumption, such as consumption by poultry such as laying hens, chicken, cow, pig, goat, horse, sheep, dog, cat, rabbit, etc. Consequently, a feed supplement as defined in the present invention should preferably have a texture, taste and smell, but also a nutritional value, that makes the supplement suitable for animal consumption.

In embodiments of the present invention a food supplement or a feed supplement with cholesterol-lowering properties comprises R. rubrum cells.

A food supplement or a feed supplement according to the invention preferably contains between 0.01% and 99.9% (w/w) of R. rubrum cells, for example 0.05%-50% by weight based on the total weight of the food supplement or the feed supplement. Preferably, a food supplement or a feed supplement contains between 10% and 90% (w/w), or between 30% and 75% (w/w), of a preparation of R. rubrum cells.

To make a food supplement or a feed supplement comprising R. rubrum cells suitable for consumption, components are preferably added to improve, for instance, texture, taste or smell.

Consequently, a food supplement or a feed supplement according to the invention preferably comprises (additional) sources of protein, carbohydrate and fat, and vitamins, minerals, electrolytes, trace elements, and other suitable components, so that the food supplement or the feed supplement itself is suitable for use as a nourishing food.

As a source of protein each and every protein that is suitable for use in nutritional formulations, and mixtures of these, are preferably used in a food supplement or a feed supplement according to the invention. This type of proteins encompasses for instance animal proteins such as whey proteins, whey protein concentrates, whey powder, egg protein, egg albumin, casein, or milk albumin, and plant proteins such as soy protein, soy meal, or proteins from soy milk. For choosing the source of proteins to be used, the biological value of a protein may constitute an important criterion. Caseinate, including calcium caseinate, but also whey, milk albumin, egg albumin, and total egg proteins, for instance, are proteins with a very high biological value, because they contain a large proportion of essential amino acids.

Suitable carbohydrates to be used in a food supplement or a feed supplement according to the invention are, for instance, preferably simple short-chain carbohydrates such as mono- and disaccharides, but also polysaccharides, or a combination of both. A carbohydrate is preferably selected because of its suitable organoleptic properties, according to the invention. Preferably, a complex carbohydrate is suitably used as a food fiber, according to the invention.

A food supplement or a feed supplement according to the invention preferably contains, in some embodiments, combinations of both simple and complex carbohydrates. A food supplement or a feed supplement according to the invention preferably contains, in some embodiments, a fat selected from all edible oils and edible fats.

Vitamins and minerals are preferably added to a preparation according to the invention, in conformity with the rules of the regulatory health authorities, and preferably encompasses all vitamins and minerals endorsed by the above authorities, for instance vitamin A, B1, B2, B12, C, D, B, and K, and folic acid, niacin, pantothenic acid, and biotin. As minerals for instance iron, zinc, iodine, calcium, magnesium, chromium, and selenium are preferably added to a preparation according to the invention.

Electrolytes such as the ions of sodium, potassium, and chloride, and trace elements and other additives do preferably also form part of a food supplement or a feed supplement according to the invention. Such components are, if present, preferably used in the recommended concentrations. Additionally, a food supplement or a feed supplement according to the invention preferably contains components improving its texture, colorings and flavorings, aromatic substances, spices, fillers, emulsifiers, stabilizing compounds, preservatives, antioxidants, fibers, and other supplements such as amino acids, choline, lecithin, fatty acids, etc. The choice of such components depends upon formulation, design, and preferences. The amounts of such components that are added are known to the skilled person, while the choice of the amounts to be added are preferably guided by for example considering the recommended daily amounts (RDA) for children and adults when a food supplement of the invention is considered.

Emulsifiers are preferably added to stabilize the final product of the invention. Examples of acceptable emulsifiers are lecithin (e.g., derived from soy or from egg), and/or mono- and di-glycerides, according to the invention. As stabilizers, carob, guar or carrageenan are, for instance, preferably used, according to the invention.

Preservatives are preferably added to increase the shelf life of the product of the invention.

Preferably, preservatives such as sodium sorbate, potassium sorbate, potassium benzoate, sodium benzoate, or calcium disodium EDTA are used in a preparation of the invention.

In addition to the carbohydrates mentioned above, natural or synthetic sweeteners, such as saccharides, cyclamates, aspartame, acesulfame potassium, and/or sorbitol, are preferably added to the food supplement or to the feed supplement, according to the invention.

The amounts of food supplement or of feed supplement of the invention to be consumed are varying in size, and are not necessarily restricted to the dosages mentioned in the dosages advised. The term “food supplement” is not meant to be restricted to a specified weight, or to a specified dose of the food supplement. The term “feed supplement” is not meant to be restricted to a specified weight, or to a specified dose of the feed supplement.

The composition of a food supplement or a feed supplement according to the invention takes in principle any form that is suitable for human or animal consumption, according to the invention.

In a preferred embodiment of the invention, the food supplement or the feed supplement is a dry powder that is suitable to be suspended, dispersed or emulsified in an aqueous solution such as water, milk, coffee, tea, broth, and fruit juice. To that end, the powder is preferably supplied in a dispenser according to the invention.

In an alternative preferred embodiment of the invention, the food supplement or the feed supplement is formulated, starting from dry powder, as a tablet or as a granulate. To this end, preferably the composition of a food supplement or a feed supplement according to the invention is suitably supplied with fillers such as microcrystalline cellulose (MCC) and mannitol, binders such as hydroxylpropyl-cellulose (HPC), lubricants such as stearic acid, and other excipients.

A food supplement or a feed supplement according to the invention is in one embodiment preferably supplied as a fluid, in which the solid components have been suspended, dispersed or emulsified. Such a composition of the invention is preferably directly mixed into a foodstuff or a feedstuff, or is preferably for instance extruded and formatted into granules or other forms.

In an alternative embodiment of the invention, a food supplement or a feed supplement is preferably formulated in a solid form, such as a bar, a biscuit, or a roll.

A food supplement or a feed supplement of the invention is preferably formulated for oral consumption, preferably in combination with an acceptable carrier such as a capsule, a tablet, a granulate, a water-miscible powder, or another form acceptable for administration. Alternatively, a food supplement of the invention is preferably processed into a foodstuff, according to the invention. Alternatively, a feed supplement of the invention is preferably processed into a feedstuff, according to the invention.

One aspect of the present invention relates to a foodstuff comprising a food supplement according to the invention. The food supplement comprising R. rubrum cells. One aspect of the present invention relates to a feedstuff comprising a feed supplement according to the invention. The feed supplement comprising R. rubrum cells.

A food supplement or a feed supplement may suitably be used to reduce intestinal cholesterol absorption, thus reducing the cholesterol level of blood plasma, in particular the plasma LDL-cholesterol concentration, while preferably leaving the HDL-cholesterol concentration essentially unaltered or raising the plasma HDL-cholesterol concentration in a subject, such that the plasma levels of HDL-cholesterol increase in absolute sense or relative to the plasma LDL-cholesterol concentration. The subject being a human subject or an animal subject such as a chicken or laying hen. The invention relates to a food supplement with cholesterol-lowering properties, comprising R. rubrum cells, such as dried R. rubrum cells obtained with refractive drying of freshly cultured R. rubrum cells.

Feeding an animal such as a broiler, hen, pig, cow, duck, goat, goose, turkey, bovine calf, sheep, in particular a pig, a broiler and a laying hen, with the feed supplement or the feed stuff comprising the feed supplement, results in the lowering of the cholesterol level in the blood serum (plasma) of said animal and/or the lowering of the cholesterol content in the meat of said animal and/or in other parts or products derived from said animal and/or in the lowering of the cholesterol content in the eggs laid by the e.g. chicken, duck, goose, turkey, etc. This lowering of the cholesterol content, in particular the LDL-cholesterol content provides for e.g. meat, eggs, etc., suitable for human consumption, and comprising a lower content of cholesterol, in particular LDL-cholesterol. Therewith, the human diet comprises less cholesterol, in particular less LDL-cholesterol, when the consumed meat and/or eggs by the human subject are derived from animals fed with the feed supplement or the feedstuff of the invention. As a consequence, less cholesterol is transported from the intestine to the blood circulation, the liver and further organs of the human subject.

In an embodiment, a food supplement of the invention is applied in a foodstuff with cholesterol-lowering properties. In an embodiment, a feed supplement of the invention is applied in a feedstuff with cholesterol-lowering properties.

A method to prepare a cholesterol-lowering foodstuff or feedstuff of the invention involves the production of a foodstuff or feedstuff, respectively, incorporating a food supplement or a feed supplement according to the invention. Such a method preferably involves a step in which a foodstuff or a feedstuff is first prepared in the normal way, followed by the addition of (dried) R. rubrum cells (obtained with refractive drying by applying the freshly cultured cells in a refraction dryer) to the prepared foodstuff or feedstuff. Also, it is possible to add (dried) R. rubrum cells to the foodstuff or to the feedstuff during its production.

A foodstuff with cholesterol-lowering properties according to the invention or a feedstuff with cholesterol-lowering properties according to the invention contains typically between 0.1% and 20% (w/w), preferably between 1% and 10% (w/w), of the food supplement or feed supplement according to the invention and described above. The feedstuff is for example chicken feed or feed for laying hen. The invention relates to a foodstuff comprising a food supplement, wherein the food supplement has cholesterol-lowering properties and comprises (dried) R. rubrum cells, the cells preferably obtained using refractive drying of freshly grown bacterial cells.

As said, atherosclerotic coronary heart disease (CHD) represents the major cause for death and cardiovascular morbidity in the Western world. Risk factors for atherosclerotic coronary heart disease include hypertension, diabetes mellitus, family history, male gender, cigarette smoke and serum cholesterol. A total cholesterol level in excess of 225-250 mg/dl is associated with significant elevation of risk of CHD.

Cholesteryl esters are a major component of atherosclerotic lesions and the major storage form of cholesterol in arterial wall cells. Formation of cholesteryl esters is also a key step in the intestinal absorption of dietary cholesterol. Thus, inhibition of cholesteryl ester formation and reduction of serum cholesterol is likely to inhibit the progression of atherosclerotic lesion formation, decrease the accumulation of cholesteryl esters in the arterial wall, and block the intestinal absorption of dietary cholesterol.

The regulation of whole-body cholesterol homeostasis in human subjects involves the regulation of dietary cholesterol and modulation of cholesterol biosynthesis, bile acid biosynthesis and the catabolism of the cholesterol-containing plasma lipoproteins. The liver is the major organ responsible for cholesterol biosynthesis and catabolism and for this reason, it is a prime determinant of plasma cholesterol levels. The liver is the site of synthesis and secretion of very low density lipoproteins (VLDL) which are subsequently metabolized to low density lipoproteins (LDL) in the circulation. LDL are the predominant cholesterol-carrying lipoproteins in the plasma and an increase in their concentration is correlated with increased atherosclerosis.

When intestinal cholesterol absorption is reduced, by whatever means, as a consequence less cholesterol is delivered to the liver. The consequence of this action is decreased hepatic lipoprotein (VLDL) production and an increase in the hepatic clearance of plasma cholesterol, mostly as LDL. Thus, the net effect of inhibiting intestinal cholesterol absorption is a decrease in plasma cholesterol levels. The inventors established that the mode of action of the dried R. rubrum cells is exactly this: limiting the amount of cholesterol absorbed in the intestine, such that LDL-cholesterol levels in the blood of human subjects decreases.

The inhibition of cholesterol biosynthesis by 3-hydroxy-3-methylglutaryl coenzyme A (HMG CoA) reductase (EC1.1.1.34) inhibitors is an effective way to reduce plasma cholesterol and treating human subjects with such inhibitors reduces atherosclerosis at least to some extent. Combination therapy of an HMG CoA reductase inhibitor and a bile acid sequestrant is more effective in human hyperlipidemic patients than either agent in monotherapy.

The daily hypocholesteremic dose of R. rubrum cells is about 0.1 g to about 60 g, or about 1 mg to about 100 mg of the active compound(s) in R. rubrum responsible for the LDL-cholesterol lowering activity by inhibiting cholesterol absorption in the intestine, or about 3 mg R. rubrum cells per kg of body weight per day to about 300 mg R. rubrum cells per kg of body weight per day, preferably about 6 mg/kg of body weight per day to about 150 mg/kg, more preferably about 12-75 mg R. rubrum cells per kg body weight per day, either provided as a single dose once daily, or provided as multiple doses at more than one time point during the day. For an average body weight of 70 kg, the dosage level is from about 50 mg R. rubrum cells to about 120 g of per day, given in a single dose or 2-4 divided doses, preferably two divided doses taken during lunch time and taken during dinner time. The exact dose, however, is determined by the attending clinician and is dependent on the potency of the R. rubrum administered, the age of the human subject, the weight, condition and response of the (healthy) human subject to the intake of R. rubrum cells.

For the combinations of this invention wherein the R. rubrum cells are administered in combination with a cholesterol biosynthesis inhibitor and/or ezetimibe, the typical daily dose of the cholesterol biosynthesis inhibitor is 0.1 to 80 mg/kg of mammalian weight per day administered in single or divided dosages, usually once or twice a day: for example, for HMG CoA reductase inhibitors, about 10 mg to about 40 mg per dose is given 1 to 2 times a day, giving a total daily dose of about 10 mg to 80 mg per day.

Where the components of a combination of R. rubrum cells and a further active ingredient are administered separately, the number of doses of each component given per day may not necessarily be the same, e.g. where one component may have a greater duration of activity, and will therefore need to be administered less frequently.

Since the present invention relates to the reduction of LDL-cholesterol levels by treatment with R. rubrum cells alone or by treatment with a combination of active ingredients wherein said active ingredients may be administered separately, the invention also relates to combining separate pharmaceutical compositions in kit form. That is, a kit is contemplated wherein two separate units are combined: R. rubrum cells and a cholesterol biosynthesis inhibitor pharmaceutical composition and/or a hydroxy substituted azetidinone cholesterol absorption inhibitor pharmaceutical composition, the hydroxy substituted azetidinone cholesterol absorption inhibitor preferably being ezetimibe. The kit preferably includes directions for the administration of the separate components. The kit form is particularly advantageous when the separate components must be administered in different dosage forms (e.g. oral and parenteral) or are administered at different dosage intervals.

A twelfth aspect of the invention relates to a kit comprising: (i) a container containing Rhodospirillum rubrum cells of any one of the embodiments of the invention, preferably dried Rhodospirillum rubrum cell granules, or the food supplement of the invention, or the foodstuff of the invention; (ii) instructions for use by a human subject; and (iii) optionally at least one pharmaceutical composition comprising any of the active pharmaceutical ingredients of embodiments of the invention, preferably a statin and/or ezetimibe; and (iv) optionally a container with a liquid, preferably water or a drink, the liquid for mixing the provided Rhodospirillum rubrum cells in the container before intake by a human subject, or the liquid for sequential or concurrent intake with the Rhodospirillum rubrum cells by a human subject.

An aspect of the invention relates to a kit comprising in separate containers in a single package pharmaceutical compositions for use in combination to treat or prevent atherosclerosis or to reduce plasma LDL-cholesterol level which comprises in a first container a pharmaceutical composition comprising an effective amount of a cholesterol biosynthesis inhibitor in a pharmaceutically acceptable carrier, preferably a statin, and in a second container, a pharmaceutical composition comprising an effective amount of a R. rubrum cells in a pharmaceutically acceptable carrier or dried R. rubrum cell granules according to any of the embodiments of the invention.

An aspect of the invention relates to a kit comprising:
a container containing Rhodospirillum rubrum cells of any one of the here above outlined embodiments of the invention or the pharmaceutical composition for use according to the invention or the food supplement of the invention, or the foodstuff of the invention;
instructions for use by a human subject; and
optionally a container with a liquid, preferably water or a drink, the liquid for mixing the provided Rhodospirillum rubrum cells in the container before intake by a human subject, or the liquid for sequential or concurrent intake with the Rhodospirillum rubrum cells by a human subject.

A further aspect of the invention relates to the Rhodospirillum rubrum cells according to the invention, a food supplement comprising the Rhodospirillum rubrum cells, a food stuff comprising the Rhodospirillum rubrum cells, or a pharmaceutical composition comprising the Rhodospirillum rubrum cells, wherein the serum concentration for at least one health-related parameter selected from ALT, AST, γGT, creatinine, NT-ProBNP, vWF, c-Troponin T, plasma glucose, hsCRP, and/or the value for SBP and/or DBP and/or resting heart rate, remains within the standard normal (healthy) range after administration of the Rhodospirillum rubrum cells or a composition comprising said cells to a human subject in need thereof, preferably all the health-related parameters including the blood pressures and resting heart rate remain in the standard normal range, when compared to the serum concentration (plasma concentration for glucose), blood pressure(s) and resting heart rate before first administration of the cells to the human subject are compared to the serum concentration, blood pressure(s) or resting heart rate after/during subsequent administration of the cells, and/or when the serum/plasma concentration, the blood pressure(s), and the resting heart rate at a first day at which the cells are administered to the human subject are compared with the serum/plasma concentration, blood pressure(s) or resting heart rate determined at a subsequent later day at which the cells are administered to the human subject.

A further aspect of the invention relates to the Rhodospirillum rubrum cells according to the invention, a food supplement comprising the Rhodospirillum rubrum cells, a food stuff comprising the Rhodospirillum rubrum cells, or a pharmaceutical composition comprising the Rhodospirillum rubrum cells, wherein the plasma glucose concentration and/or the serum concentration for at least one health-related parameter selected from ALT, AST, γGT, creatinine, NT-ProBNP, vWF, c-Troponin T, hsCRP, remains within the standard normal (healthy) range after administration of the (composition comprising) Rhodospirillum rubrum cells to a human subject in need thereof, preferably all the health-related parameters remain in the standard normal range, when compared to the serum or plasma concentration before first administration of the cells to the human subject are compared to the serum/plasma concentration after the first or subsequent administration of the cells, and/or when the serum/plasma concentration at a first day at which the cells are administered to the human subject are compared with the serum/plasma concentration at a subsequent later day at which the cells are administered to the human subject.

A further aspect of the invention relates to the Rhodospirillum rubrum cells according to the invention, a food supplement comprising the Rhodospirillum rubrum cells, a food stuff comprising the Rhodospirillum rubrum cells, or a pharmaceutical composition comprising the Rhodospirillum rubrum cells, wherein the values for SBP and/or DBP remain within the standard normal (healthy) range after (first or subsequent) administration of the Rhodospirillum rubrum cells to a human subject in need thereof, preferably the values for SBP and/or DBP remain essentially unaltered, when the blood pressure(s) before first administration of the cells to the human subject is/are compared to the blood pressure(s) after the first administration of the cells, and/or when the blood pressure(s) at a first day at which the cells are administered to the human subject is/are compared with the blood pressure(s) determined at a subsequent later day at which the cells are administered to the human subject.

A further aspect of the invention relates to the Rhodospirillum rubrum cells according to the invention, a food supplement comprising the Rhodospirillum rubrum cells, a food stuff comprising the Rhodospirillum rubrum cells, or a pharmaceutical composition comprising the Rhodospirillum rubrum cells, wherein the values for resting heart rate remains within the standard normal (healthy) range after (first or subsequent) administration of the Rhodospirillum rubrum cells to a human subject in need thereof, preferably the values for resting heart rate remain essentially unaltered, when the resting heart rate before first administration of the cells to the human subject is compared to the resting heart rate after the first administration of the cells, and/or when the resting heart rate at a first day at which the cells are administered to the human subject is compared with the resting heart rate determined at a subsequent later day at which the cells are administered to the human subject.

An embodiment is the Rhodospirillum rubrum cells for use according to the invention, wherein the serum values for one or more of AST, ALT, γGT, creatinine, NT-ProBNP and hsCRP essentially remain unaltered in the blood serum of said human subject. An embodiment is the Rhodospirillum rubrum cells for use according to the invention, wherein the serum concentrations for one or more of AST, ALT, γGT, creatinine, NT-ProBNP and hsCRP essentially remain within standard normal or healthy boundaries in the blood serum of said human subject. An embodiment is the Rhodospirillum rubrum cells for use according to the invention, wherein the values for SBP and/or DBP remain essentially unaltered or remain within standard normal, healthy boundaries for said subject, when the SBP and the DBP are determined before and during the administration of the cells to said human subject and compared, and/or when the SBP and the DBP are determined at a first day at which the cells are administered to said human subject and at a second day at which the cells are administered to said human subject, and compared. An embodiment is the Rhodospirillum rubrum cells for use according to the invention, wherein the values for resting heart rate remains essentially unaltered or remains within standard normal, healthy boundaries for said subject, when the resting heart rate is determined before and during the administration of the cells to said human subject and compared, and/or when the resting heart rate is determined at a first day at which the cells are administered to said human subject and at a second day at which the cells are administered to said human subject, and compared.

### EMBODIMENTS OF THE INVENTION

Embodiment 1: Rhodospirillum rubrum cells for use in a method for lowering of LDL-cholesterol concentration in blood of a human subject.

Embodiment 2: Rhodospirillum rubrum cells for use in the treatment or the prophylaxis of a cardiovascular disease in a human subject.

Embodiment 3: Rhodospirillum rubrum cells for use in the treatment or prophylaxis of atherosclerosis, dyslipidemia, arteriosclerosis, hypercholesterolemia, familial hypercholesterolemia, hyperlipidemia, an LDL-cholesterol plasma level of at least 70 mg/dL, an LDL-cholesterol plasma level of at least 100 mg/dL, an LDL-cholesterol plasma level of at least 140 mg/dL, an LDL-cholesterol plasma level of at least 200 mg/dL, a total plasma cholesterol level of at least 200 mg/dL, a total plasma cholesterol level of 5.0 mM-8.0 mM, a plasma Lp(a) level of at least 14 mg/dL, ischemia, in a human subject.

Embodiment 4: Rhodospirillum rubrum cells for use in the inhibition of absorption of cholesterol from the intestine of a human subject, such that LDL-cholesterol concentration in the blood of said human subject decreases or is decreased.

Embodiment 5: Rhodospirillum rubrum cells for use according to Embodiment 4, wherein the HDL-cholesterol concentration in the blood of the human subject remains unaltered or increases or is increased.

Embodiment 6: Non-therapeutic method of lowering LDL-cholesterol concentration in blood of a human subject, the method comprising administering to the human subject a composition comprising of or consisting of Rhodospirillum rubrum cells.

Embodiment 7: Non-therapeutic method of treating or preventing a cardiovascular disease in a human subject, the method comprising administering to the human subject a composition comprising of or consisting of Rhodospirillum rubrum cells.

Embodiment 8: Non-therapeutic method of treating or preventing atherosclerosis, dyslipidemia, arteriosclerosis, hypercholesterolemia, familial hypercholesterolemia, hyperlipidemia, homozygous sitosterolemia, an LDL-cholesterol plasma level of at least 70 mg/dL, an LDL-cholesterol plasma level of at least 100 mg/dL, an LDL-cholesterol plasma level of at least 140 mg/dL, an LDL-cholesterol plasma level of at least 200 mg/dL, a total plasma cholesterol level of at least 200 mg/dL, a total plasma cholesterol level of 5.0 mM-8.0 mM, a plasma Lp(a) level of at least 14 mg/dL, ischemia, in a human subject, the method comprising administering to the human subject a composition comprising of or consisting of Rhodospirillum rubrum cells.

Embodiment 9: The non-therapeutic method according to any one of the Embodiments 6-8, wherein the administration to the human subject of the composition comprising of or consisting of Rhodospirillum rubrum cells inhibits absorption of cholesterol from the intestine of the human subject, such that LDL-cholesterol concentration in the blood of said human subject decreases or is decreased, and preferably such that HDL-cholesterol concentration in the blood of the human subject remains unaltered or increases or is increased.

Embodiment 10: The Rhodospirillum rubrum cells for use according to any one of the Embodiments 1-5 or the non-therapeutic method according to any one of the Embodiments 6-9, wherein the Rhodospirillum rubrum cells are administered to a human subject having an LDL-cholesterol level in plasma of at least 1.8 mmol/L (70 mg/dL), or at least 2.59 mmol/L (100 mg/dL), or at least 3.34 mmol/L (129 mg/dL), or at least 4.0 mmol/L, such as at least 5.2 mmol/L (200 mg/dL) or between 5.0 mM and 8.0 mM.

Embodiment 11: The Rhodospirillum rubrum cells for use according to any one of the Embodiments 1-5, 10 or the non-therapeutic method according to any one of the Embodiments 6-10, wherein Rhodospirillum rubrum cells are administered to a human subject to whom at least one active pharmaceutical ingredient is (co-)administered, such as at least one active pharmaceutical ingredient selected from a statin, niacin, fenofibrate, ezetimibe, colesevelam, mipomersen, lomitapide, a PCSK9 inhibitor, alirocumab, evolocumab, ETC-1002, a CETP inhibitor, anacetrapib, evacetrapib, WAY-252623, a blood-pressure lowering compound, hydrochlorothiazide, preferably a statin such as atorvastatin, fluvastatin, pravastatin, rosuvastatin, simvastatin, lovastatin and/or ezetimibe.

Embodiment 12: The Rhodospirillum rubrum cells for use according to Embodiment 11 or the non-therapeutic method according to Embodiment 11, wherein the statin and/or the ezetimibe are administered to the subject at a lower total daily dose when co-administered with Rhodospirillum rubrum cells, compared to the daily dose during standard therapeutic treatment with the statin or with the ezetimibe.

Embodiment 13: The Rhodospirillum rubrum cells for use according to any one of the Embodiments 1-5, 10-12 or the non-therapeutic method according to any one of the Embodiments 6-12, wherein the Rhodospirillum rubrum cells are administered orally to the human subject.

Embodiment 14: The Rhodospirillum rubrum cells for use according to any one of the Embodiments 1-5, 10-13 or the non-therapeutic method according to any one of the Embodiments 6-13, wherein the Rhodospirillum rubrum cells are administered to a healthy human subject, preferably a healthy human subject with a total cholesterol concentration in the blood before the first administration of Rhodospirillum rubrum cells, of at least 5.0 mM, such as 5.0 mM-8.0 mM.

Embodiment 15: The Rhodospirillum rubrum cells for use according to any one of the Embodiments 1-5, 10-14 or the non-therapeutic method according to any one of the Embodiments 6-14, wherein administering the Rhodospirillum rubrum cells to a human subject results in lowering of the plasma LDL-cholesterol concentration, preferably to a plasma concentration of less than 3.34 mmol/L, preferably less than 2.59 mmol/L, more preferably to a plasma LDL-cholesterol concentration of less than 1.8 mmol/L.

Embodiment 16: The Rhodospirillum rubrum cells for use according to any one of the Embodiments 1-5, 10-15 or the non-therapeutic method according to any one of the Embodiments 6-15, wherein administering the Rhodospirillum rubrum cells to a human subject results in a decrease of the LDL-cholesterol concentration in the plasma of said human subject, wherein the plasma HDL-cholesterol concentration remains essentially unaltered or decreases to a smaller extent than the decrease in the plasma LDL-cholesterol concentration, or wherein the plasma HDL-cholesterol concentration increases, preferably the plasma HDL-cholesterol concentration remains essentially unaltered or increases.

Embodiment 17: The Rhodospirillum rubrum cells for use according to any one of the Embodiments 1-5, 10-16 or the non-therapeutic method according to any one of the Embodiments 6-16, wherein administering the Rhodospirillum rubrum cells to a human subject results in a decrease of the plasma LDL-cholesterol concentration in said subject with at least 1%, preferably at least 5%, more preferably at least 6%, more preferably at least 10%, most preferably at least 20%, based on the plasma LDL-cholesterol concentration prior to the administration of the Rhodospirillum rubrum cells to said human subject.

Embodiment 18: The Rhodospirillum rubrum cells for use according to any one of the Embodiments 1-5, 10-17 or the non-therapeutic method according to any one of the Embodiments 6-17, wherein administering the Rhodospirillum rubrum cells to a subject results in a decrease of the plasma LDL-cholesterol concentration to, or maintenance of the plasma LDL-cholesterol concentration at, a plasma LDL-cholesterol concentration of less than 200 mg/dL, or less than 159 mg/dL, or less than 129 mg/dL, preferably less than 100 mg/dL, such as at or less than 70 mg/dL.

Embodiment 19: The Rhodospirillum rubrum cells for use according to any one of the Embodiments 1-5, 10-18 or the non-therapeutic method according to any one of the Embodiments 6-18, wherein the Rhodospirillum rubrum cells are formulated as an oral dosage form, preferably a solid oral dosage form or a liquid oral dosage form, preferably a liquid oral dosage form comprising an oil or a drink.

Embodiment 20: The Rhodospirillum rubrum cells for use according to any one of the Embodiments 1-5, 10-19 or the non-therapeutic method according to any one of the Embodiments 6-19, wherein the Rhodospirillum rubrum cells are formulated as a granulate, preferably a granulate provided in a capsule such as a gelatin capsule or in a sachet.

Embodiment 21: The Rhodospirillum rubrum cells for use according to any one of the Embodiments 1-5, 10-20 or the non-therapeutic method according to any one of the Embodiments 6-20, wherein the Rhodospirillum rubrum cells are dried Rhodospirillum rubrum cell granules obtained by subjecting freshly cultured cells to refractive drying at 55° C.-100° C., preferably at 60° C.-100° C., more preferably at 80° C.-100° C., such as 56-95° C., 57-90° C., 58-85° C., 59-80° C., 60-75° C., and 61-70° C., 60-70° C., 62-69° C. and 63-68° C.

Embodiment 22: The Rhodospirillum rubrum cells for use according to any one of the Embodiments 1-5, 10-21 or the non-therapeutic method according to any one of the Embodiments 6-21, wherein a daily dose of Rhodospirillum rubrum cells to be administered to a human subject is 100 mg-50 g per day, preferably 200 mg-25 g per day, more preferably 500 mg-10 g per day, most preferably 1 g-5 g per day, such as 0.25 g, 0.50 g, 1.0 g, 2.0 g, 3.0 g, 4.0 g, 5.0 g, 7.5 g, 15 g, 20 g, 30 g, or 40 g.

Embodiment 23: The Rhodospirillum rubrum cells for use according to any one of the Embodiments 1-5, 10-22 or the non-therapeutic method according to any one of the Embodiments 6-22, wherein a daily dose of Rhodospirillum rubrum cells to be administered to a human subject is at least 1.0 g, such as at least 5.0 g, preferably 1.0-100 g, more preferably 1.0-50 g, most preferably 5.0-20 g.

Embodiment 24: The Rhodospirillum rubrum cells for use according to any one of the Embodiments 1-5, 10-23 or the non-therapeutic method according to any one of the Embodiments 6-23, wherein the Rhodospirillum rubrum cells are dosed to provide an amount of between 0.1 g and 50 g of Rhodospirillum rubrum cells per day.

Embodiment 25: The Rhodospirillum rubrum cells for use according to any one of the Embodiments 1-5, 10-24 or the non-therapeutic method according to any one of the Embodiments 6-24, wherein a daily dose of Rhodospirillum rubrum cells to be administered to a human subject are two half-daily doses each comprising half of the daily dose, wherein preferably a first half-daily dose is administered 1-2 hours before lunch, 1-2 hours after lunch or during lunch taken by the human subject, and a second half-daily dose is administered 1-2 hours before dinner, 1-2 hours after dinner or during dinner taken by the human subject, preferably during lunch and during dinner.

Embodiment 26: The Rhodospirillum rubrum cells for use according to any one of the Embodiments 1-5, 10-25 or the non-therapeutic method according to any one of the Embodiments 6-25, wherein administering the Rhodospirillum rubrum cells to a subject maintains the LDL-cholesterol concentration in the plasma of the subject at a level of less than 159 mg/dL, preferably less than 129 mg/dL, more preferably less than 100 mg/dL, most preferably at or less than 70 mg/dL, such as between 50 mg/dL and 159 mg/dL.

Embodiment 27: Pharmaceutical composition comprising a pharmaceutically effective amount of Rhodospirillum rubrum cells and optionally a pharmaceutically acceptable excipient, for use in a method for the lowering of LDL-cholesterol in blood plasma of a human subject.

Embodiment 28: Pharmaceutical composition comprising a pharmaceutically effective amount of Rhodospirillum rubrum cells and optionally a pharmaceutically acceptable excipient, for use in a method for the treatment or prophylaxis of any one or more of cardiovascular disease, atherosclerosis, dyslipidemia, arteriosclerosis, hypercholesterolemia, familial hypercholesterolemia, hyperlipidemia, homozygous sitosterolemia, an LDL-cholesterol plasma level of at least 70 mg/dL, an LDL-cholesterol plasma level of at least 100 mg/dL, an LDL-cholesterol plasma level of at least 140 mg/dL, an LDL-cholesterol plasma level of at least 200 mg/dL, a total plasma cholesterol level of at least 200 mg/dL, a total plasma concentration in the blood of at least 5.0 mM such as between 5.0 mM and 8.0 mM, an Lp(a) level of at least 14 mg/dL, inflammation, inflammatory disease, ischemia, infection.

Embodiment 29: Pharmaceutical composition for use according to Embodiment 27 or 28, wherein the Rhodospirillum rubrum cells are the sole active pharmaceutical ingredient in said pharmaceutical composition.

Embodiment 30: Pharmaceutical composition for use according to Embodiment 27 or 28, wherein the pharmaceutical composition is administered to a subject to whom at least one further active pharmaceutical ingredient is administered, such as an active pharmaceutical ingredient selected from a statin, niacin, fenofibrate, ezetimibe, colesevelam, mipomersen, lomitapide, a PCSK9 inhibitor, alirocumab, evolocumab, ETC-1002, a CETP inhibitor, anacetrapib, evacetrapib, WAY-252623, a blood-pressure lowering compound, hydrochlorothiazide, or any combination thereof such as a statin and a PCSK9 inhibitor or a statin and ezetimibe, preferably a statin such as atorvastatin, fluvastatin, pravastatin, rosuvastatin, simvastatin, lovastatin, and/or ezetimibe.

Embodiment 31: Pharmaceutical composition for use according to any one of the Embodiments 27-30, wherein the Rhodospirillum rubrum cells are administered to the human subject as replacement therapy such as therapy replacing a statin and/or ezetimibe, or are administered to the human subject in combination with a lower dose of (a) LDL-cholesterol lowering pharmaceutical compound(s) administered to the human subject than the dose of such (a) LDL-cholesterol lowering pharmaceutical compound(s) administered to the human subject before administration of Rhodospirillum rubrum cells started.

Embodiment 32: Pharmaceutical composition for use according to any one of the Embodiments 27-31, wherein the pharmaceutical composition is administered to a human subject suffering from an LDL-cholesterol plasma level of higher than 70 mg/dL, an LDL-cholesterol plasma level of at least 100 mg/dL, an LDL-cholesterol plasma level of at least 140 mg/dL, an LDL-cholesterol plasma level of at least 200 mg/dL, a total cholesterol concentration in the blood of at least 5.0 mM such as 5.0 mM-8.0 mM, a cardiovascular disease, hypercholesterolemia, atherosclerosis.

Embodiment 33: Pharmaceutical composition for use according to any one of the Embodiments 27-32, wherein the Rhodospirillum rubrum cells are the Rhodospirillum rubrum cells of any one of the Embodiments 13, 20-25.

Embodiment 34: Food supplement with cholesterol-lowering properties when ingested by a human subject, comprising Rhodospirillum rubrum cells, wherein the Rhodospirillum rubrum cells are dried Rhodospirillum rubrum cell granules obtained by subjecting freshly cultured cells to refractive drying at 55° C.-100° C., preferably at 60° C.-100° C., more preferably at 60° C.-80° C.

Embodiment 35: Foodstuff comprising a food supplement according to Embodiment 34.

Embodiment 36: The food supplement of Embodiment 34 or the foodstuff of Embodiment 35, wherein the Rhodospirillum rubrum cells are the Rhodospirillum rubrum cells of any one of the Embodiments 13, 20-25.

Embodiment 37: Kit comprising:
a container containing Rhodospirillum rubrum cells of any one of the Embodiments 13, 20-25, preferably dried Rhodospirillum rubrum cell granules, or the food supplement of Embodiment 34, or the foodstuff of Embodiment 35;
instructions for use by a human subject; and
optionally at least one pharmaceutical composition comprising any of the active pharmaceutical ingredients of Embodiments 11, 12, 30, 31, preferably a statin and/or ezetimibe; and
optionally a container with a liquid, preferably water or a drink, the liquid for mixing the provided Rhodospirillum rubrum cells in the container before intake by a human subject, or the liquid for sequential or concurrent intake with the Rhodospirillum rubrum cells by a human subject.

Embodiment 38: Rhodospirillum rubrum cells according to any one of the Embodiments 13, 20-25, the pharmaceutical composition according to Embodiment 27 or 29, the food supplement according to Embodiment 34 or the foodstuff according to Embodiment 35, wherein a value for at least one parameter selected from ALT, AST, γGT, creatinine, NT-ProBNP, vWF, c-Troponin T, plasma glucose, hsCRP, remains within normal range in the serum of a human subject to whom the cells are administered after administration or during daily administration of the Rhodospirillum rubrum cells to said human subject, preferably all the values for said parameters remain within the normal range and more preferably remain essentially unaltered.

ALT, AST, γGT and creatinine are preferably measured using a spectrophotometric assay. c-troponin T and NT-PRoBNP are preferably measured using an immunoassay. vWF is preferably measured using ELISA. hsCRP is preferably measured using an immunoturbidimetric assay.

Embodiment 39: Rhodospirillum rubrum cells according to any one of the Embodiments 13, 20-25, the pharmaceutical composition according to Embodiment 27 or 29, the food supplement according to Embodiment 34 or the foodstuff according to Embodiment 35, wherein DBP and/or SBP, is within normal range for a human subject to whom the cells are administered after administration or during daily administration of the Rhodospirillum rubrum cells to said human subject, preferably DBP and/or SBP remain essentially unaltered.

DBP and SBP are preferably measured using an Omron M7 (Omron Healthcare Co. Ltd., Kyoto, Japan) (See Examples section, here above).

Embodiment 40: Rhodospirillum rubrum cells according to any one of the Embodiments 13, 20-25, the pharmaceutical composition according to Embodiment 27 or 29, the food supplement according to Embodiment 34 or the foodstuff according to Embodiment 35, wherein the resting heart rate is within normal range for a human subject to whom the cells are administered after administration or during daily administration of the Rhodospirillum rubrum cells to said human subject, preferably the resting heart rate remains essentially unaltered.

Resting heart rate is preferably measured using an Omron M7 (Omron Healthcare Co. Ltd., Kyoto, Japan) (See Examples section, here above).

The present invention has been described above with reference to a number of exemplary embodiments as shown in the drawings. Modifications and alternative implementations of some parts or elements are possible, and are included in the scope of protection as defined in the appended claims.

### EXAMPLES

### Measurement of Cholesterol Levels

### Total Cholesterol Assay—Mouse Plasma

The measurement of total plasma cholesterol in the mouse plasma was conducted using the in vitro kit “cholesterol” from Beckman Coulter Nederland B.V. (Woerden, Netherlands, Datasheet from 09-2011, coded BAOSR6x16.02; catalogue No. OSR6116) intended for the quantitative determination of Cholesterol concentrations in serum, EDTA plasma, heparinized plasma on Beckman Coulter AU analyzers. Reference is made to the protocol used as provided by Beckman Coulter reagent kit.

Measurements of cholesterol are used primarily in the diagnosis and treatment of disorders involving excess cholesterol in the blood, and lipid and lipoprotein metabolism disorders. Total serum or plasma cholesterol analysis has proven useful in the diagnosis of hyperlipoproteinemia, atherosclerosis, hepatic and thyroid diseases. Total and HDL cholesterols, in conjunction with a triglyceride determination, provide valuable information for the prediction of coronary heart disease.

### Method & Materials

Mouse plasma was used.

The system reagent of the kit (OSR6116/OSR6216/OSR6516) was obtained from Beckman Coulter Nederland B.V. and consists of:
Phosphate buffer (pH 6.5)—103 mmol/L
Cholesterol Esterase (Candida/Pancreatic)—≥0.2 kU/L (3.3 μkat/L)
4-Aminoantipyrine—0.31 mmol/L
Cholesterol Oxidase (Brevibacterium)—≥0.2 kU/L (3.3 μkat/L)
Phenol—5.2 mmol/L
Peroxidase (Horseradish)—≥10.0 kU/L (166.7 μkat/L)
Preservatives

The Cholesterol reagents are ready for use. No preparation was required.

### Methodology

The reagent is brought into contact with the plasma in aBeckman Coulter analyzer, which causes the cholesterol esters in the serum to be hydrolyzed by the cholesterol esterase (CHE). The free cholesterol produced in this reaction is oxidized by cholesterol oxidase (CHO) to cholest-4-en-3-one with the simultaneous production of hydrogen peroxide (H₂O₂), which oxidatively couples with the 4-aminoantipyrine and phenol in the presence of the peroxidase to yield a chromophore. The red quinoneimine dye formed through this reaction can be measured spectrophotometrically at 540/600 nm as an increase in absorbance.

Measurements obtained typically fall within these risk categories, when a human subject is considered:
<200 mg/dL—normal total cholesterol level
200-239 mg/dL—bordering on high total cholesterol level
>240 mg/dL—high total cholesterol level

### Triglycerides Assay—Mouse Plasma

The measurement of plasma triglyceride levels in the mouse plasma was conducted using the in vitro kit “tryglycerides” from Beckman Coulter Nederland B.V. (Woerden, Netherlands, Datasheet from 11-2010, coded BAOSR6x118.02; catalogue No. OSR60118) intended for the quantitative determination of Triglyceride concentrations in human serum, EDTA, or heparinized plasma samples on Beckman Coulter AU analyzers. Reference is made to the protocol used as provided by Beckman Coulter reagent kit.

Triglycerides are the major form of fat found in nature and their primary function is to provide energy for the cell. Measurements of triglyceride are used in the diagnosis and treatment of patients with diabetes mellitus, nephrosis, liver obstruction, other diseases involving lipid metabolism, or various endocrine disorders. Clinically, triglyceride assays are used to help classify the various genetic and metabolic lipoprotein disorders and in the assessment of risk factors for atherosclerosis and coronary artery disease.

### Method & Materials

Mouse plasma was used.

The system reagent of the kit (OSR60118/OSR61118/OSR66118) was obtained from Beckman Coulter Nederland B.V. and consists of:
PIPES buffer (pH 7.5)—50 mmol/L
Lipase (Pseudomonas)—≥1.5 kU/L (25 μkat/L)
Glycerol kinase (Bacillus stearothermophilus)—≥0.5 kU/L (8.3 μkat/L)
Glycerol phosphate oxidase (Pseudomonas)—≥1.5 kU/L (25 μkat/L)
Ascorbate oxidase (Curcubita species)—≥1.5 kU/L (25 μkat/L)
Peroxidase (Horseradish)—≥0.98 kU/L (16.3 μkat/L)
ATP—1.4 mmol/L
4-Aminoantipyrine—0.50 mmol/L
Magnesium acetate—4.6 mmol/L
MADB—0.25 mmol/L
Preservatives

For OSR60118 and OSR61118, the Triglyceride Reagents are ready for use. No preparation was required. For OSR66118, the pipe supplied had to be inserted into the 180 mL reagent vial before use on the analyzer. The pipe was for single use only.

### Methodology

This triglyceride procedure is based on a series of coupled enzymatic reactions. The triglycerides in the sample are hydrolyzed by a combination of microbial lipases to give glycerol and fatty acids. The glycerol is phosphorylated by adenosine triphosphate (ATP) in the presence of glycerol kinase (GK) to produce glycerol-3-phosphate. The glycerol-3-phosphate is oxidized by molecular oxygen in the presence of GPO (glycerol phosphate oxidase) to produce hydrogen peroxide (H₂O₂) and dihydroxyacetone phosphate. The formed H₂O₂ reacts with 4-aminophenazone and N,N-bis(4-sulfobutyl)-3,5-dimethylaniline, disodium salt (MADB) in the presence of peroxidase (POD) to produce a chromophore, which is read at 660/800 nm. The increase in absorbance at 660/800 nm is proportional to the triglyceride content of the sample.

Measurements obtained typically fall within these risk categories, when a human subject is considered:
<150 mg/dL—Normal
150-199 mg/dL—Borderline High
200-499 mg/dL—High
≥500 mg/dL—Very High

### LDL Cholesterol Assay—Mouse Plasma

The measurement of plasma LDL cholesterol in the mouse plasma was conducted using the in vitro kit “LDL-cholesterol” from Beckman Coulter Nederland B.V. (Woerden, Netherlands, Datasheet from 08-2009, coded BAOSR6x96.01; catalogue No. OSR6196) intended for the quantitative determination of LDL-Cholesterol concentrations in human serum, EDTA, or heparinized plasma samples on Beckman Coulter AU analyzers. Reference is made to the protocol used as provided by Beckman Coulter reagent kit.

Measurements of cholesterol are used primarily in the diagnosis and treatment of disorders involving excess cholesterol in the blood, and lipid and lipoprotein metabolism disorders. LDL-Cholesterol plays a causal role in the development of coronary heart disease (CHD). In 1988 the National Cholesterol Education Program Adult Treatment Panel (NCEP-ATP) developed recommendations for the diagnosis and treatment of patients with hypercholesterolemia. These recommendations defined LDL-Cholesterol as the primary target of therapy. The 2001 update of these guidelines (NCEP-ATP III) put further emphasis on better risk identification and more aggressive cholesterol-lowering treatment.

### Method & Materials

Mouse plasma was used.

The system reagent of the kit (OSR6196/OSR6296) was obtained from Beckman Coulter Nederland B.V. and consists of:
MES Buffer (pH 6.3)
Cholesterol esterase (Pseudomonas)—1875 U/L
Cholesterol oxidase (Nocardia)—1125 U/L
Peroxidase (Horseradish)—975 U/L
Detergent 1—0.75%
Detergent 2—0.25%
DSBmT—0.25 mmol/L
4-aminoantipyrine—0.375 mmol/L
Ascorbate Oxidase—2250 U/L
Preservative

The Cholesterol reagents are ready for use. No preparation was required.

### Methodology

The LDL-Cholesterol test is a two reagent homogenous system. The assay is comprised of two distinct phases. In phase one a unique detergent solubilizes cholesterol from non-LDL-lipoprotein particles. This cholesterol is consumed by cholesterol esterase, cholesterol oxidase, peroxidase and 4-aminoantipyrine to generate a colorless end product.

In phase two a second detergent in the reagent releases cholesterol from the LDL—lipoproteins. This cholesterol reacts with cholesterol esterase, cholesterol oxidase and a chromogen system to yield a blue color complex which can be measured bichromatically at 540/660 nm. The resulting increase in absorbance is directly proportional to the LDL-C concentration in the sample.

The guidelines (NCEP-ATP III) classify LDL—Cholesterol levels as follows, when a human subject is considered:
1. <100 mg/dL—Optimal
2. 100-129 mg/dL—Near optimal/above optimal
3. 131-159 mg/dL—Borderline high
4. 160-189 mg/dL—High
5. ≥190 mg/dL—Very high

### HDL Cholesterol Assay—Mouse Plasma

The measurement of plasma HDL cholesterol in the mouse plasma was conducted using the in vitro kit “HDL-cholesterol” from Beckman Coulter Nederland B.V. (Woerden, Netherlands, Datasheet from 08-2009, coded BAOSR6x95.01; catalogue No. OSR6195) intended for the quantitative determination of HDL-Cholesterol concentrations in human serum, EDTA, or heparinized plasma samples on Beckman Coulter AU analyzers. Reference is made to the protocol used as provided by Beckman Coulter reagent kit.

Measurements of cholesterol are used primarily in the diagnosis and treatment of disorders involving excess cholesterol in the blood, and lipid and lipoprotein metabolism disorders. Many epidemiological investigations have demonstrated the strong and independent inverse association between HDL-Cholesterol and the risk of coronary artery disease. It has been proposed that HDL particles, through the uptake and transport of Cholesterol from peripheral tissue to the liver (reverse Cholesterol transport), protects against the development of atheromatous plaques. Under the guidelines issued by The National Cholesterol Education Program Adult Treatment Panel 2 (NCEP ATP 2) it is recommended that both HDL-Cholesterol and Total Cholesterol should be measured in the initial screening for hypercholesterolemia.

### Method & Materials

Mouse plasma was used.

The system reagent of the kit (OSR6195/OSR6295) was obtained from Beckman Coulter Nederland B.V. and consists of:
Goods Buffer (pH 6.0)
Cholesterol esterase (Pseudomonas)—375 U/L
Cholesterol oxidase (E. coli)—750 U/L
Peroxidase (Horseradish)—975 U/L
Ascorbate oxidase (Curcubita sp.)—2250 U/L
DSBmT—0.75 mmol/L
4-aminoantipyrine—0.25 mmol/L
Detergent—0.375%
Preservative—0.05%

The Cholesterol reagents are ready for use. No preparation was required.

### Methodology

The HDL-Cholesterol test is a two reagent homogenous system for the selective measurement of serum or plasma HDL-Cholesterol in the presence of other lipoprotein particles. The assay is comprised of two distinct phases. In phase one, free cholesterol in non-HDL-lipoproteins is solubilized and consumed by cholesterol oxidase, peroxidase, and DSBmT to generate a colorless end product.

In phase two a unique detergent selectively solubilizes HDL-lipoproteins. The HDL cholesterol is released for reaction with cholesterol esterase, cholesterol oxidase and a chromogen system to yield a blue color complex which can be measured bichromatically at 600/700 nm. The resulting increase in absorbance is directly proportional to the HDL-C concentration in the sample.

The guidelines (NCEP ATP 2) classify HDL-C levels as follows, when a human subject is considered:
1. <40 mg/dL as indicative of a major risk factor for Coronary Heart Disease.
2. >60 mg/dL as a negative risk factor for Coronary Heart Disease.

### Example 1

Plasma LDL-Cholesterol Lowering Activity of Freeze-Dried R. rubrum Cells in Mice

Dried Rhodospirillum rubrum bacteria, 25 g, were purchased from Algosource Technologies (Saint-Nazaire, France) and stored at 4° C. until use.

Eight-week old male mice (21-27 g) were of the C57BL/6J strain that is well known for their high cholesterol level upon exposure to a high fat (a so-called Western) diet. The mice were obtained from Charles River Laboratories (France).

The feed for the mice was obtained from Altromin Spezialfutter GmbH (Lage, Germany). Hydrogels were obtained from ClearH2O (Westbrook, USA) and contained approximately 65 gram of 97% water in a gel.

The 65 g hydrogel was heated for 30 minutes at 70° C. and then 1.5 g control diet (in the pretest diet and in the diet for the control group), or 1.5 g dried bacteria (the diet for the treated group) was gently mixed into the liquefied hydrogel with a spatula. Subsequently, 13.5 g high fat diet was also mixed into the hydrogel. The mixing was continued until a smooth homogenous product was obtained. The hydrogel was weighed before and after mixing with the diets and also just prior to placement into the animal cages. The hydrogel-diets were stored in the dark at 4° C. for a maximum of 3 days.

The mice were placed in cages with litter underneath a mesh. Enrichment was provided in the form of a polymer shelter and a piece of paper towel. The mice were provided the hydrogel-diet cups as their only source of food and water.

Upon reception from the supplier the mice were quarantined together for 7 days. After individually housing, the mice were provided the feed-hydrogel mix in cups containing 60 gr hydrogel, 13.5 gr high fat diet and 1.5 g control diet for 7 days. The feed-hydrogel product was refreshed 3 times over that period.

After 7 days two groups were formed: a treatment group (n=4) receiving the feed-hydrogel mix in cups containing 13.5 g high fat diet and 1.5 g dried R. rubrum bacteria for 7 days and a control group (n=4) receiving the feed-hydrogel mix in cups containing 13.5 g high fat diet and 1.5 g control diet for 7 days. Again, hydrogel-diet product was refreshed 3 times over that period.

Every second day the mice and the remaining hydrogel-diet cup were weighed to assess the effect of the two feeds on the mice weight development and their food consumption.

At the end of the second week the mice were weighed and anaesthetized with 0.05 ml of 200 mg/ml Nembutal (Kela, Lot 26344A13). After sufficient sedation the chest cavity was opened, and blood was taken with a 24 G needle on a 1 ml syringe (both heparin-treated) and stored in a 2 ml Vacutainer EDTA tube on ice.

The blood was centrifuged for 10 minutes at 1500 rpm at 4° C. to separate the cells from the plasma. The plasma was stored at 4° C. overnight.

The cholesterol was measured in the plasma by company SynLab (Mons, Belgium). In this study subcontractor SynLab was chosen to perform the cholesterol analysis using an ELISA-based method that required less plasma for their measurements.

Statistical significance was tested in Microsoft Excel with Student's t-test (unpaired, equal size, equal variance).

### Results

The goal was to examine the effect of administering Rhodospirillum rubrum (GEPEA/Algosolis) on the blood cholesterol level in mice. The feed was mixed into hydrogel and was offered ad libitum. All mice are comprised in the pre-test (n=8). After one week the mice were divided into the control group (same feed as pretest, n=4) and the treated group (feed with R. rubrum bacteria cells, n=4) and kept on these respective feeds for one week.

For one mouse (no. 6) not enough blood was obtained, and subsequently not enough plasma resulted.

After the second week the treated group (n=4) showed a slight weight gain of 1.75 g (±0.35 g) whereas the control group (n=4)−1.5 g (±1.37 g) showed a weight loss (p=0.0018). This difference occurred after the second week (the treatment week), but was not present after the first week (pre-test: all 8 mice).

The weekly food consumption by the mice showed no difference between the consumption of the groups in the second week or as compared to the first week (pre-test: all 8 mice).

Total plasma cholesterol levels for treated mice that were administered R. rubrum cells were 6% lower than those of the control group with a p value of 0.047 (statistically significant; p-values below 0.05 when compared to the corresponding control parameter).

LDL cholesterol levels of the treated mice that were administered R. rubrum cells, were 39% lower than those of the control group with a p value of 0.044 (statistically significant; p-values below 0.05 when compared to the corresponding control parameter).

Non-HDL cholesterol levels (mostly comprised of LDL and VLDL) of the treated mice were 32% lower than those of the control group with a p value of 0.053.

In conclusion, feeding mice with feed comprising Rhodospirillum rubrum bacteria cells produced by GEPEA/Algosolis had a statistically significant reductive effect on the total plasma cholesterol levels (−6%) and on the LDL cholesterol levels (−39%) of C57BL/6J mice placed on a high fat diet.

The species of bacterium is from the genus Rhodospirillum, or is a mixture of different Rhodospirillum spp. selected for example from Rhodospirillum rubrum, Rhodospirillum centenum, Rhodospirillum photometricum, Rhodospirillum oryzae, Rhodospirillum sulfurexigens, Rhodospirillum salexigens, Rhodospirillum salinarum, Rhodospirillum sodomense, and Rhodospirillum tenue. For example, the Rhodospirillum is Rhodospirillum rubrum.

### Example 2—Effect of Freeze-Dried R. rubrum Cells on Chow-Fed Rats

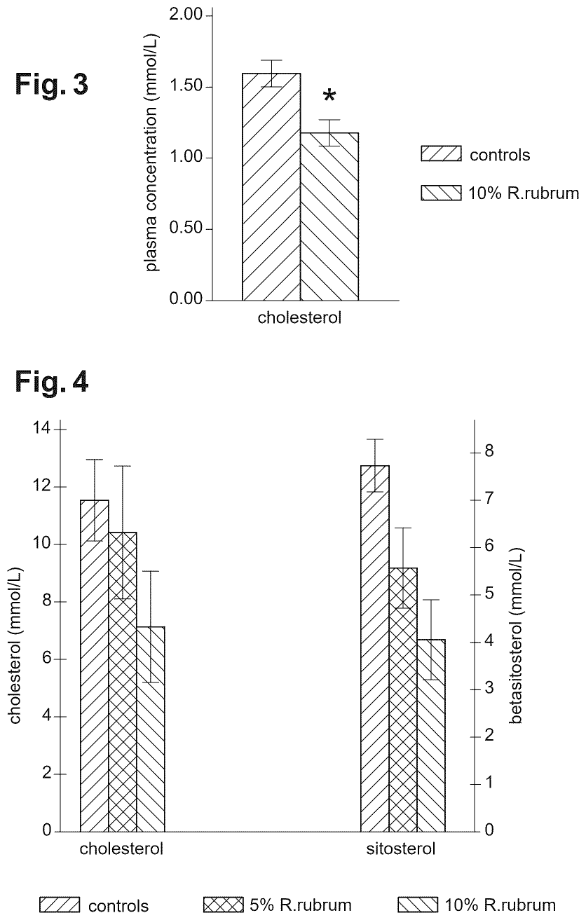

Male Wistar rats were fed a semi-synthetic rat chow, meeting nutritional requirements (Hope Farms, Woerden, the Netherlands). One group of eight rats was given this basic chow. A second group of eight rats was given the same chow, but now containing in addition 10% (w/w) of freeze-dried R. rubrum cells (in exchange for sucrose). Both groups consumed approximately the same amount of food (31±7 gram/day), and showed the same increase in body weight over time. After eight weeks, all clinical chemistry-parameters measured (plasma glucose, uric acid, urea, creatinine, GOT, GPT, haematocrit, and haemoglobin, and urinary glucose and protein) were similar in both groups, except plasma cholesterol (FIG. 3) and plasma triglycerides. Plasma cholesterol was significantly lower in the group fed the R. rubrum-containing diet (1.2±0.1 mmol/L vs. 1.6±0.1 mmol/L; t-test, p<0.0001) (FIG. 3), as were plasma triglycerides (0.5±0.1 mmol/L vs. 1.4±0.6 mmol/L; p<0.001). Separation of the plasma lipoproteins by fast-protein-liquid chromatography (fplc, ÅKTA system of Pharmacia-Amersham) showed that the decrease in cholesterol and triglycerides was due to a decrease of the plasma LDL fraction, while the HDL fraction remained unchanged in the R. rubrum-fed animals. The R. rubrum-induced decrease in plasma cholesterol was thus specifically due to a decrease in LDL-cholesterol.

### Example 3 Effect of Freeze-Dried R. Rubrum in Mice Fed a “Western-Type” Diet

Ten C57Black/6 mice were fed for three weeks a semi-synthetic diet, the so-called “Western-type” diet, a diet containing 15% (w/w) fat, and 0.25% (w/w) cholesterol. Subsequently, five mice were fed the same diet for seven days, while another five mice were fed the same diet but containing in addition 10% (w/w) R. rubrum (some cholesterol was also added to this diet to keep its cholesterol content at 0.25%). After these seven days, the cholesterol level in the control group was 3.07±0.18 mmol/L, while in the group fed the diet containing R. rubrum the cholesterol level was 2.26±0.21 mmol/L (t-test, p=0.0003) (FIG. 4). Plasma triglycerides were not decreased. Separation of the lipoproteins by FPLC, showed that the LDL-cholesterol had practically disappeared from the plasma of mice fed the Western-type diet containing R. rubrum, while the HDL-cholesterol showed no inter-group difference (FIG. 2).

### Example 4 Effect of Freeze-Dried R. Rubrum in Transqenic APOE*3Leiden Mice Fed a “Western-Type” Diet

In this experiment mice were used in which the human gene for the so-called Leiden mutation of apolipoprotein E3 (APOE*3Leiden) had been incorporated by transgenesis. Because of this transgenic change, these so-called APOE*Leiden mice have a humanized lipoprotein profile, and are extremely suitable for studying the effect of compounds on lipoprotein metabolism.

The study design was as follows: groups of mice were fed for 5 weeks “Western-type” diet (see above) containing 0.25% (w/w) cholesterol. This diet increased their plasma cholesterol level to 13-14 mmol/L. Subsequently, the mice were randomized, on the basis of their plasma cholesterol level, into groups of six mice each. These groups were given the same diet, but containing in addition 0, 0.625, 1.25, 2.5, 5 or 10% (w/w) freeze-dried R. rubrum. The cholesterol content of the diet was kept at 0.25% by adding cholesterol as required.

During the experiment, body weight and food intake were monitored. After the dietary change, blood was taken weekly to determine plasma cholesterol and triglyceride levels. Also, the plasma lipoprotein pattern was determined group-wise in pooled samples by FPLC. In addition, faeces were collected weekly on a group basis. After three weeks, VLDL secretion was measured in the groups fed 0% and 10% R. rubrum (terminal experiment). The group fed 0.625% R. rubrum was then changed to a diet containing 0% R. rubrum, the group fed 2.5% R. rubrum to 5% R. rubrum, and the group fed 5% R. rubrum to 10% R. rubrum, to replace the two groups sacrificed. After another week, blood was again sampled from the four remaining groups.

The results can be summarized as follows:
the plasma cholesterol level was significantly reduced in the groups fed 5% or 10% R. rubrum
cholesterol lowering was already very significant after one week in the 10% R. rubrum group (p<0.0001), and after two weeks also in the 5% R. rubrum group (p<0.001), and remained so for the three-week period
this lower cholesterol level was due to a lowering of cholesterol in the VLDL and LDL fractions, while the amount of cholesterol in the HDL fractions did not change

The plasma levels of camposterol and β-sitosterol, two sterols that occur only in plants and can thus only be present in plasma after uptake from the intestines of the mice, decreased significantly in the mouse groups fed either 5%, or 10% R. rubrum (FIG. 4). The ratio of the plasma concentration of β-sitosterol to cholesterol did not change significantly (ANOVA, p=0.26), and neither did the ratio of plasma camposterol to cholesterol. (ANOVA, p=0.98).

Since the plasma concentration of cholesterol is determined by its rate of synthesis and its rate of absorption from the intestines, the fact that these ratios remained similar (cholesterol to camposterol, β-sitosterol) demonstrates that the decrease in the plasma cholesterol concentration cannot be due to a decrease in cholesterol synthesis, but must rather be ascribed to an decreased absorption of sterols from the intestinal lumen of the mice.

### In Summary:

These experiments show that adding 5% (w/w) or 10% (w/w) of R. rubrum cells to a “Western-type” diet for mice reduces, in APOE*3Leiden mice, plasma cholesterol levels significantly. This decrease
1. is ascribed to a decrease of cholesterol carried in (pro-atherogenic) VLDL and LDL particles, while (anti-atherogenic) HDL cholesterol remains unchanged;
2. is not caused by a decrease in cholesterol synthesis, since it was observed that plasma lathosterol levels did not change, while the synthesis/secretion of VLDL by the liver was also unchanged;
3. is not due to an increased excretion of bile acids, which this is only slightly increased; but
4. is therefore due to a decreased absorption of sterols from the intestines, as reflected by observed increased excretion of cholesterol in the faeces, and the decrease in plasma camposterol and β-sitosterol concentrations.

The cholesterol-lowering effect is caused by at least two different strains of R. rubrum (ATCC 25903 and DSM 467).

### Example 5

Effect of Carotenoids, Pheophytins and/or (Ubi)Quinols/(Ubi)Quinones on Plasma LDL-Cholesterol Levels in Mice

### Experimental Procedures

The Rhodospirillum rubrum strain S1H was stored in liquid nitrogen in a 10% w/w sucrose-0.85% w/w saline solution. To regrow the strain, the cells were taken out the liquid nitrogen and thawed for 30 minutes at room temperature. Cells were streaked on a sistrom succinate agar plate and a rich Luria Bertani (LB) medium to grow colony forming units. The agar plates were incubated at 30° C. in dark and aerobic conditions for up to 4 days.

After 4 days, 10 single colonies were picked up and transferred to 10 tubes with 2 mL of Sistrom succinate liquid medium and incubated at 30° C. in dark, aerobic and orbital shaking at 150 rpm. After 4-5 days, the cells were grown and reached an OD₆₈₀=0.5-0.6. To check the axenicity of the cultures, the cells were streaked on a sistrom succinate agar plate and a rich LB medium agar plate and incubated up to 1 week to look for heterotrophic contaminants. When the axenicity check was approved, the 2 mL cultures were transferred to 15 mL of Sistrom succinate liquid medium and incubated at 30° C. in dark, aerobic and orbital shaking at 150 rpm. After 4-5 days the cells were grown and reached an OD₆₈₀=0.5-0.6. Then, the 15 mL cultures were transferred to 100 mL of Sistrom succinate liquid medium and incubated at 30° C. in dark, aerobic and orbital shaking at 100 rpm. After 4-5 days, the cells were grown and reached an OD₆₈₀=0.5-0.6. Once the axenicity was checked on Sistrom succinate and LB medium, these cells constituted the inoculum cultures.

### Cell Culturing

### Culture Conditions

### Bioreactor

Light anaerobic conditions were applied for culturing the Rhodospirillum rubrum strain S1H cells. Ten axenic inoculum cultures were pelleted by centrifugation at 5000*g for 10 minutes. The supernatant was discarded and the pellets were pooled in 25 mL of Melissa liquid medium with acetate as carbon source to constitute a concentrated inoculum.

The bioreactor was sterilized by wet-heat sterilization, 20 minutes of water vapor exposure at 121° C. and 1.2 bar in an autoclave. After sterilization the bioreactor was closed. Bottles with Melissa medium, 1 M H₂SO₄, and for the effluent were coupled aseptically in a laminar flow cabinet (LAF).

### Extraction Protocols

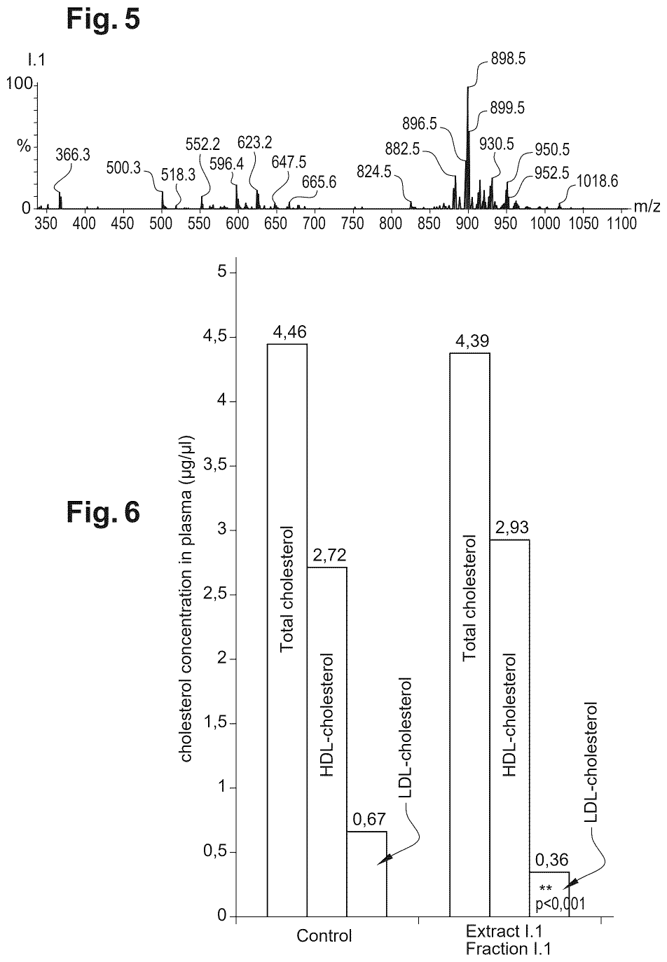

30 g of bacterial pellet of Rhodospirillum rubrum strain S1H cells, are mixed using 440 ml of a biphasic mixture of petroleum ether (boiling point 60-80° C.) and methanolic saline during 2 h at room temperature (biphasic mixture: 220 ml containing 20 ml of NaCl 0.3% by mass and 200 ml of methanol+220 ml of the petroleum ether (boiling point 60-80° C.)). After centrifugation at 5000 RPM during 20 minutes at room temperature, the upper phase (the petroleum ether phase) was removed and stored at room temperature. Lower phase (the methanolic saline solution) was optionally submitted to a second extraction with an additional 220 ml of petroleum ether for 2 h at room temperature. The second upper phase was mixed with the first one and dried using a rotavapor system. The resulting viscous liquid was named “FRACTION l.1”, or “fl.1”, or “Extract l.1” in FIGS. 5 and 6.

### Materials and Methods for Mass Spectrometry Analysis

### MALDI-ToF

Matrix-assisted laser desorption ionization time-of-flight (MALDI-ToF) mass spectrum was recorded using a QToF Premier mass spectrometer equipped with a Nd:YAG laser, operating at 355 nm with a output frequency of 50 Hz. Time-of-flight mass analyses were performed in reflection mode at a resolution of about 10.000. Samples of fl.1 were analyzed using (DCTB)trans-2-[3-(4-tertbutylphenyl)-2-methylprop-2-enylidene]malononitrile. This matrix was prepared as a 40 mg/mL solution in CHCl₃. The matrix solution (1 mg/mL) was applied to a stainless steel target and air dried. The samples were dissolved in THE and I microliter aliquots of this solution were applied onto the target area already bearing the matrix crystals and air dried. For the recording of the single-stage MS spectra, the quadrupole (rf-only mode) was set to pass ions from 200 to 2500 Th, and all ions were transmitted into the pusher region of the time-of-flight analyzer where they were mass analyzed with 1 s integration time.

Q-tOF (5600 ABSCIEX)—nanoESI-MS

Samples of fl.1 were diluted in 0.1% formic acid in acetonitrile, centrifuged at room temperature during 5 min at 13.000 RPM and the supernatants were infused directly in the Mass spectrometer (flow rate: 89 microliter/hour) using nano-esi source. The acquisition parameters were: ion source gas1:4; Curtain gas 15; ionspray Voltage floating 2.300, heater temperature 150° C.; Polarity positive; ToF mass range: 100-2.000.

Ion Trap (HCT Ultra Brucker)—nanoESI-MS

Samples of fl.1 were diluted in 0.1% formic acid in acetonitrile, centrifuged at room temperature during 5 minutes at 13.000 RPM and the supernatants were infused directly in the Mass spectrometer (flow rate: 89 microliter/hour) using nano-esi source. The acquisition parameters were: capillary 1.900 Volt; Dry gas: 6 I/min; Dry temp: 250° C.; Polarity: positive; scan mode Standard—enhanced; scan range: 100-2.000; Smart target 20.000; Max accu time: 200 ms.

### Results

With MALDI-ToF analyses of fl.1, the presence of the carotenoids rhodovibrin, 1-hydroxyspirilloxanthin, 3,4-didehydro-rhodopin, chloroxanthin, rhodopin, spirilloxanthin, and 3,4-dihydro-spirilloxanthin, as well as the (ubi)quinones and (ubi)quinoles ubiquinol-10, ubiquinone-9, ubiquinone-10, and rhodoquinone-10, as well as the bacteriopheophytins a, geranylgeranyl bacteriopheophytin a, and phytyl derivative of bacteriopheophytin a in fl.1 was determined (molecular ion indicated as M+H+ value was 585.5 for rhodovibrin; 583.5 for 1-hydroxy-spirilloxanthin; 587.5 for 3,4-dehydro-rhodopin; 557.5 for chloroxanthin; 555.4 for rhodopin; 597.4 for spirilloxanthin; 599.5 for 3,4-dihydro-spirilloxanthin; 865.7 for ubiquinol-10; 795.6 for ubiquinone-9; 848.7 for ubiquinone-10; 863.7 for rhodoquinone-10; 883.5 for geranyl-geranyl bacteriopheophytin a; 889.5 for phytyl bacteriopheophytin a, respectively) (FIG. 5). The carotenoids rhodovibrin, 1-hydroxyspirilloxanthin, 3,4-didehydro-rhodopin, chloroxanthin, rhodopin, spirilloxanthin, and 3,4-dihydro-spirilloxanthin, as well as the (ubi)quinones and (ubi)quinoles ubiquinol-10, ubiquinone-9, ubiquinone-10, and rhodoquinone-10, as well as the bacteriopheophytins a, geranylgeranyl bacteriopheophytin a, and phytyl derivative of bacteriopheophytin a were also identified in the fl.1 extract when applying nano-ESI Q-ToF analysis. Ten μl of the fraction l.1 was dried in presence of matrix.

Analysis of the fl.1 extract using Q-tOF (5600 ABSCIEX)—nanoESI-MS confirmed the presence of the carotenoids rhodovibrin, 1-hydroxyspirilloxanthin, 3,4-didehydro-rhodopin, chloroxanthin, rhodopin, spirilloxanthin, and 3,4-dihydro-spirilloxanthin, as well as the (ubi)quinones and (ubi)quinoles ubiquinol-10, ubiquinone-9, ubiquinone-10, and rhodoquinone-10, as well as the bacteriopheophytins a, geranylgeranyl bacteriopheophytin a, and phytyl derivative of bacteriopheophytin a present in fl.1. NanoESI MS spectra were obtained for the fraction l.1 with a triple tof mass spectrometer (ABSCIEX) using acetonitrile 99%, 1% HCOOC as organic solvent.

In the Q-tOF (5600 ABSCIEX)—nanoESI-MS spectrum for fl.1 with carotenoids rhodovibrin, 1-hydroxyspirilloxanthin, 3,4-didehydro-rhodopin, chloroxanthin, rhodopin, spirilloxanthin, and 3,4-dihydro-spirilloxanthin, as well as the (ubi)quinones and (ubi)quinoles ubiquinol-10, ubiquinone-9, ubiquinone-10, and rhodoquinone-10, as well as the bacteriopheophytins a, geranylgeranyl bacteriopheophytin a, and phytyl derivative of bacteriopheophytin a, peaks were revealed at the following m/z values (approximate relative intensity in brackets): 647.6 (2.5); 651.6 (3.3); 881.5 (2); 927.5(1).

NanoESI MS spectra are obtained for the fraction l.1 with an IonTrap mass spectrometer (Bruker) using 1% HCOOC in acetonitrile.

Mice Test—Effect of Administering Petroleum-Ether Fraction of R. Rubrum Cells, ‘Fl.1’, with Carotenoids Rhodovibrin, 1-Hydroxyspirilloxanthin, 3,4-Didehydro-Rhodopin, Chloroxanthin, Rhodopin, Spirilloxanthin, and 3,4-Dihydro-Spirilloxanthin, as Well as the (Ubi)Quinones and (Ubi)Quinoles Ubiquinol-10, Ubiquinone-9, Ubiquinone-10, and Rhodoquinone-10, as Well as the Bacteriopheophytins a, Geranylgeranyl Bacteriopheophytin a, and Phytyl Derivative of Bacteriopheophytin a on Plasma Cholesterol Level

The mice test for testing the influence of a diet comprising fl.1 with carotenoids rhodovibrin, 1-hydroxyspirilloxanthin, 3,4-didehydro-rhodopin, chloroxanthin, rhodopin, spirilloxanthin, and 3,4-dihydro-spirilloxanthin, as well as the (ubi)quinones and (ubi)quinoles ubiquinol-10, ubiquinone-9, ubiquinone-10, and rhodoquinone-10, as well as the bacteriopheophytins a, geranylgeranyl bacteriopheophytin a, and phytyl derivative of bacteriopheophytin a on plasma cholesterol level was performed at SCK⋅CEN animalarium (BE) following 2 weeks acclimation of 40 C57BL/6 male mice. After initial weighing of the food and the mice, they were placed in individual ventilated cage. Food consumption was checked every day and hydrogel weighed every 2 days. Based on previously performed preliminary palatability tests, the bacterial extracts were resuspended in sunflower oil, and 5% regular sugar was added to the chow (Cafeteria-diet) to ensure high palatability.

The first week, the first group of mice received the Cafeteria Diet+sunflower oil replacing R. rubrum extract fl.1 ad libitum while the second group of mice received the Cafeteria Diet+the control diet replacing R. rubrum extract fl.1 ad libitum.

The second week, the control group continued on the same diet while the three experimental groups received the Cafeteria Diet+10% of either the fl.1 extract in sunflower oil. Thus, one group of mice was fed with feed comprising the fl.1 petroleum ether extract of Rhodospirillum rubrum, a second group of mice, the control group, was fed feed without extract of Rhodospirillum rubrum.

Effects of feeding control feed or feed comprising fl.1 comprising carotenoids rhodovibrin, 1-hydroxyspirilloxanthin, 3,4-didehydro-rhodopin, chloroxanthin, rhodopin, spirilloxanthin, and 3,4-dihydro-spirilloxanthin, as well as the (ubi)quinones and (ubi)quinoles ubiquinol-10, ubiquinone-9, ubiquinone-10, and rhodoquinone-10, as well as the bacteriopheophytins a, geranylgeranyl bacteriopheophytin a, and phytyl derivative of bacteriopheophytin a on cholesterol levels in plasma is detailed below.

### End of Mice Test

After 2 weeks of testing, the mice where weighted and euthanized using intraperitoneal pentobarbital injection prior to dissection. Whole-blood was removed in EDTA-tubes, centrifuged to obtain plasma and placed at 4° C. for further analysis.

### Mice Weight

After week 1 and week 2, no differences between the groups were detected.

### Blood Analysis

### Total Cholesterol, HDL and LDL Fractions

FIG. 6 shows the results of the cholesterol-, HDL- and LDL analysis in the blood of the treated mice.

Extract l.1 (which is Fraction l.1, fl.1) comprising the carotenoids rhodovibrin, 1-hydroxyspirilloxanthin, 3,4-didehydro-rhodopin, chloroxanthin, rhodopin, spirilloxanthin, and 3,4-dihydro-spirilloxanthin, as well as the (ubi)quinones and (ubi)quinoles ubiquinol-10, ubiquinone-9, ubiquinone-10, and rhodoquinone-10, as well as the bacteriopheophytins a, geranylgeranyl bacteriopheophytin a, and phytyl derivative of bacteriopheophytin a, has a significant effect on the plasma LDL-cholesterol concentration in mice since the Extract l.1 decreased plasma LDL-cholesterol levels in mice for more than 40%, i.e. about 46%, compared to the control group of mice (p<0.001) while the total plasma cholesterol levels stayed essentially unchanged. See FIG. 6. Furthermore, also the HDL-cholesterol level in the mice group that were fed the fl.1 fraction, stayed essentially unaltered after the experimental period. Total cholesterol in plasma was 4.39 μg/μl for the fl.1 group of mice compared to 4.46 μg/μl for the control group, HDL-cholesterol was 2.93 μg/μl for the fl.1 group and 2.72 μg/μl for the control group, and LDL-cholesterol was 0.36 μg/μl for the fl.1 group (p<0.001) and 0.67 μg/μl for the control group, respectively.

### Conclusions

From this mice test using R. rubrum S1H extracts fl.1 comprising the carotenoids rhodovibrin, 1-hydroxyspirilloxanthin, 3,4-didehydro-rhodopin, chloroxanthin, rhodopin, spirilloxanthin, and 3,4-dihydro-spirilloxanthin, as well as the (ubi)quinones and (ubi)quinoles ubiquinol-10, ubiquinone-9, ubiquinone-10, and rhodoquinone-10, as well as the bacteriopheophytins a, geranylgeranyl bacteriopheophytin a, and phytyl derivative of bacteriopheophytin a, it is determined that the petroleum ether extract fl.1 has a beneficial effect on lowering LDL-cholesterol level in mice to a large extent, while at the same time keeping the HDL-cholesterol level essentially unaltered when the extract is administered orally.

### Example 6—Testing Cholesterol Lowering Activity of Oven-Dried R. Rubrum Cells

Dried Rhodospirillum rubrum bacteria, 150 g, were purchased from AW Van Bennekom VOF (The Netherlands) and stored at 4° C. until use. Bacterium cells were oven-dried R. rubrum cells obtained with refractive drying (at 60° C.-100° C., preferably 60° C.-85° C., such as about 60° C.) before being fed to mice in the experiments detailed here below.

### Materials

The 6-week old male mice (21-27 g) chosen for this experiment were of the C57BL/6J strain that is well known for their high cholesterol level upon exposure to a high fat (a so-called Western) diet. The mice were obtained from Charles River Laboratories (France). The food was obtained from Altromin Spezialfutter GmbH& Co. KG (Lage, Germany). The hydrogels were obtained from ClearH2O (Westbrook, USA) and contained 65 gram of 97% water in a gel. The cholesterol kit (ab65390) was obtained from Abcam through VWR International (Haasrode, Belgium). Other materials were obtained from VWR International (Haasrode, Belgium).

### Methods

The 65 hydrogel was heated for 1 hr at 50° C. and then 13.5 g high fat diet was gently mixed into the liquefied hydrogel with a spatula. Subsequently 1.5 g control diet (in the pretest diet and the diet for the control group) or 1.5 g bacterial extract (the diet for the treated group) was also mixed into the hydrogel. The mixing was continued until a smooth homogenous product was obtained. The hydrogel was weighed before and after mixing with the diets and also just prior to placement into the animal cages. The hydrogel-diets were stored at 4° C. for a maximum of 3 days. The mice were placed in cages with litter underneath a mesh. Enrichment was provided in the form of polymer tubing and paper towel. The mice were only provided the hydrogel-diet cups as a source of food and water. Upon reception from the supplier the mice were quarantined for 7 days together. After individually housing the mice, they were provided the hydrogel-diet containing 60 gr hydrogel, 13.5 gr high fat diet and 1.5 g control diet for 7 days. In fact, the hydrogel-diet product was refreshed 3 times over that period. After 7 days the mice were randomized and two groups formed: a treated group receiving the hydrogel containing 13.5 g high fat diet and 1.5 g bacterial extract for 7 days and a control group (n=5) receiving the hydrogel containing 13.5 g high fat diet and 1.5 g control diet for 7 days. Again, hydrogel-diet product was refreshed 3 times over that period. Every second day the mice and the remaining hydrogel-diet was weighted to assess the effect of the bacterial extract (i.e. oven-dried R. rubrum cells obtained with refractive drying at 60° C. (refractive drying with R. rubrum cells is suitable at a temperature of 55-100° C., preferably at 60-100° C., more preferably at 80-100° C., in a refraction dryer)) on the mice weight development and their food consumption.

At the end of the second week the mice were weighed and anaesthetized with 0.1 ml of Nembutal. After sufficient sedation the chest cavity was opened and blood was taken with a 21 G needle on a 1 ml syringe (both heparin treated) and stored in a 2 ml Vacutainer EDTA tube on ice. The blood was centrifuged for 10 min at 1500 rpm at 4° C. to separate the cells from the plasma. The cholesterol was measured in the plasma according to the Abcam ab65390 kit procedure. The remaining plasma was stored at −20° C. Statistical significance was tested in Microsoft Excel with Student's t-test (unpaired, equal size, equal variance).

### Results

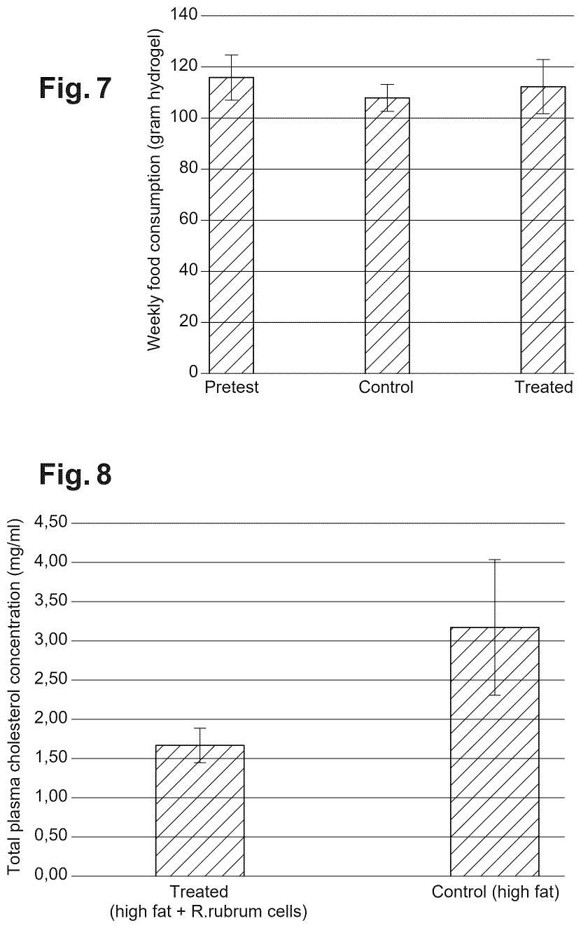

The goal of this experiment was to examine the effect on the blood cholesterol levels of oven-dried R. rubrum cells (oven-dried R. rubrum cells obtained with refractive drying at 60° C., in mice. Total plasma cholesterol levels were measured according to the procedure described in the analytical kit and a linear standard curve was obtained. The mice were randomized over the treatment groups to avoid experimental bias in the following sequence: 2 treated, 2 control, 2 treated, 2 control, 1 treated, 1 control. Weekly food consumption of the mice showed no difference between the consumption over the first week (pretest: all 10 mice) or the second week for the treated group (5 mice) or the control group (5 mice) (FIG. 7). No significant difference in weight gain was observed between the groups (1.0+/−1.4 gram for the treated and 1.6+/−0.5 gram for the control group). Total plasma cholesterol levels for treated mice were 47% lower than those of the control group with a p value of 0.0083 (FIG. 8).

### Conclusions

The treatment of mice with the bacterial product of oven-dried R. rubrum cells (oven-dried R. rubrum cells obtained with refractive drying at 60° C. has a statistically significant reductive effect (47%) on the total plasma cholesterol levels of C57BL/6J mice placed on a high fat diet. Since no significant effect was observed on weight gain or food consumption it is concluded that the product has a physiological effect on endogenous cholesterol household.

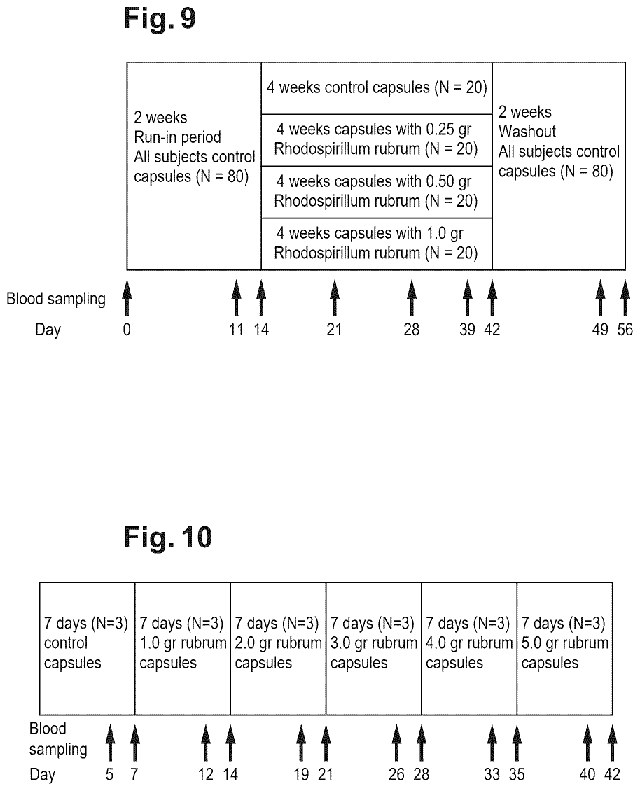

The batch of oven-dried R. rubrum cells (oven-dried R. rubrum cells obtained with refractive drying at 60° C. is used in the clinical trial described here below: treatment of healthy human male subjects with a daily dose of 0.25 g, 0.50 g or 1.00 g oven-dried R. rubrum cells take at half daily dose twice daily (treatment protocol outlined in FIG. 9). The cells were oven-dried R. rubrum cells obtained with refractive drying using a refraction dryer at 60° C.

### Example 7—Provision of R. Rubrum Cells for Assessing Effects on Cholesterol Homeostasis in Human Subjects, Tested in a Double-Blind Randomized Controlled Clinical Trial

### Physical/Chemical Properties of R. Rubrum Oven-Dried Cells

The inoculum was grown using LED lamps specifically selected for the production of Rhodospirillum rubrum. The aim was to translate underwater assimilation lighting and cultivation technology used in cultivation of freshwater algae to a technical solution for Rhodospirillum rubrum production. The results proved that Rhodospirillum rubrum can be grown with an 850 nm infrared LED lamp. In addition, the pigment concentration that coincides with cholesterol-lowering activity in animal models (rats, mice) appears to be higher in Rhodospirillum rubrum when using these infrared LED lamps. Within the production protocol a phase-step approach was used:
Phase 1, production of a small amount of Rhodospirillum rubrum at a 10 L scale;
Phase 2, optimization and scaling.

A Rhodospirillum rubrum culture was started in a 1 liter bottle, sterilizable, with an internal lamp, turbidity sensor and a batch production of 30 mg/l/day using flocculant and filtration. As a follow-up step, 4×500 ml centrifuges were placed before harvest, thereby increasing the productivity of the 1 liter reactor to 100 mg/L/day. Based on these results, the production of a 10-liter reactor with sensor, an internal lamp and an incubator (stericult) has been used to make the pre-culture more stable. Two culture systems were used for the benefit of the pre-culture. The first method consisted of 10 liter bottles with internal and external LED lighting. The second method concerned a shaker table with plastic foil bags as a culture container and used external lighting. Both systems (internally exposed bottles or external-exposed bags) are suitable for the stable production of Rhodospirillum rubrum biomass on a 10 liter scale. These systems are suitable for the production of small amounts of material (typically 1 to 100 grams/week).

Upscaling to continuous process—Based on the results with the 10 liter reactor, a reproducible productivity of 122 mg/L/day is achieved. Based hereon, production at 100 liter scale is performed.

Shaker tables to increase production are implemented in the production process. The shaker tables serve a production enhancement purpose to obtain sufficient medium for centrifugation and the Rhodospirillum rubrum can be extracted from the medium. The shaker table makes an upward and downward movement every minute, with the infrared LED lights located above the cultures. Rhodospirillum rubrum production was scaled up to a 200 liter scale. The typical final density of a batch production is between 700 mg/L and 1200 mg/L of dry matter, which depends on the duration of the cultivation. Yield per day when dry matter in mg/L culture is considered, is optimal for R. rubrum cell cultures cultured for 7 days or longer. Based on the results with the 10 L cultures and the 200 liter scale cultures, scaling up the Rhodospirillum rubrum culture to 1.000 liters and 10,000 liters is suitable.

A cell harvesting method was applied that uses flocculation. The method is robust and scalable. For the drying of Rhodospirillum rubrum biomass, a refractive drying method known in the art using a refraction dryer known in the art has been applied and used, which operates at a low temperature, uses hot water to heat up the R. rubrum cell biomass and an air stream to discard evaporated water such that the R. rubrum cell biomass is dried, wherein the drying is performed at atmospheric pressure. The hot water has a temperature typically near the boiling point or somewhat below such as 55-100° C., 60-100° C. or 80-100° C., such as 55-99° C., 58-98° C., 80-99.5° C. or 80-90° C., here typically about 55-65° C., such as 60° C., or 80-100° C., such as about 95° C. Refractive drying (oven-drying using a refraction dryer) shows the superior levels of nutrient retention, color, flavor, and aroma as compared to conventional drying methods like freeze-drying, spray-drying and drum-drying. Indeed, the refractive drying method provides stable biomass for the different experiments, i.e. for the DBRCCT. An amount of 15 kg of oven-dried Rhodospirillum rubrum cell material has been produced. The typical yield was 0.7 to 1.0 grams per liter of dry matter in a 7-day culture cycle. After oven drying in the refraction dryer the R. rubrum material (oven-dried R. rubrum cells obtained with refractive drying (for example at about 60°)), a finely granulated powder is obtained. The powder has a distinct red brown color and has the consistency and texture that resembles ground coffee. The oven-dried biomass was hermetically sealed after harvesting and stored in a refrigerator at +4° C. An in vivo small-scale mice test according to the experimental outlines as detailed in Example 6 here above was performed in order to confirm the stability and continued efficacy of the agent. Indeed, again the oven-dried R. rubrum cells lowered plasma LDL-cholesterol with at least 30-40% in the mice fed with diet comprising the R. rubrum cells (10% based on the total weight of the feed).

### Example 8—Clinical Trial with Healthy Human Subjects Treated with Dried R. Rubrum Cells

First in Human Trial Assessing the Effect of Dried R. Rubrum Cells on Plasma Cholesterol Level—Proof of Concept Study to Investigate for the First Time Whether R. Rubrum Cells Dried in a Refraction Dryer have Plasma Cholesterol Lowering Activity in Human Subjects

### Summary

Rationale: Cardiovascular diseases are still the leading cause of morbidity and mortality in the modern Western societies. Dietary interventions that aim to lower serum LDL cholesterol concentrations are important, since a high LDL cholesterol is causally related to cardiovascular risk. A reduction in serum LDL cholesterol concentrations by 10% lowers future CVD risk by 20%.

Objective: The primary objective of the proposed study is to examine for the first time the LDL cholesterol lowering effect of oven-dried Rhodospirillum rubrum (oven-dried R. rubrum cells obtained with refractive drying at 60° C. in humans. Secondary objectives are to investigate the effects on other CVD risk parameters: total cholesterol, triacylglycerol, HDL-C, glucose, blood pressure and resting heart rate. Finally, we will monitor safety parameters by weekly measurements of a panel of endpoints consisting of markers for liver (ALT, AST, γGT), and kidney function (creatinine) as well as heart function (NT-ProBNP, vWF, c-Troponin T).

Study design: The proposed study is a 4-weeks randomized, double-blind placebo-controlled trial with a parallel design using 3 doses oven-dried Rhodospirillum rubrum (oven-dried R. rubrum cells obtained with refractive drying at 60° C.).

Study population: Eighty-two (N=82) healthy men 18-75 years of age with a slightly elevated fasting serum total cholesterol concentrations (between 5.0-8.0 mmol/1) were enrolled for the study. A total of six (N=6) men did not finish the trial due to circumstances not related to the oven-dried Rhodospirillum rubrum intake.

Intervention: During the intervention period of 4 weeks, men will receive either placebo or capsules containing 0.25 gr, 0.5 gr or 1.0 gr oven-dried (oven-dried R. rubrum cells obtained with refractive drying at 60° C.) Rhodospirillum rubrum per day.

Main study parameters/endpoints: The primary endpoint is the change in serum LDL cholesterol concentrations in view of the three doses dried R. rubrum cells or placebo. Stratification based on for example age groups, or based on men having a relatively low total cholesterol concentration at the start of the intervention (e.g. 5.0-6.5 mM) versus men having a relatively high total cholesterol concentration at the start of the intervention (>6.5 mM), etc., is also part of the post-trial data analyses.

### Introduction and Rationale of the First in Man Human Clinical Trial Phase IIa

Cardiovascular diseases (CVD) are still the leading cause of morbidity and mortality in the modern Western societies. Pharmacological treatments in defined patients groups are very successful in lowering serum LDL cholesterol concentrations, a major risk factor for CVD. It is accepted that each reduction in serum LDL cholesterol by 10% lowers future CVD risk by 20%. However, a large proportion of the population does not qualify for drug treatments and these subjects are only encouraged to adapt their lifestyle as a preventive strategy to improve their metabolic profile. For CVD, (dietary) prevention programs are mainly focused on lowering LDL cholesterol.

Several successful experiments in various animal models have created the awareness that a mixture of structural compounds being part of Rhodospirillum rubrum is an interesting ingredient that might beneficially modulate cholesterol metabolism. It has been shown in different models that Rhodospirillum rubrum lowers serum LDL cholesterol concentrations which, given the well-known causal role of LDL cholesterol in atherosclerotic plaque formation, may translate into reduced cardiovascular risk when these effect would also occur when the cells are administered to human subjects in need thereof. An important issue is to show that the reducing effects on serum LDL cholesterol concentrations seen e.g. in rats and mice are also found in humans. Therefore, the inventors conducted the first proof of concept (POC) study according to the gold standard, i.e. a DBRCCT, in humans with the primary objective to show that oven-dried Rhodospirillum rubrum (oven-dried R. rubrum cells obtained with refractive drying at 60° C.) lowers serum LDL cholesterol concentrations. The second aim of this POC trial is to show that these effects on LDL cholesterol are dose dependent in apparently healthy men with slightly elevated serum cholesterol concentrations, and that oven-dried Rhodospirillum rubrum is well tolerated. Therefore, an objective of the inventors was to examine the dose-response relationship between oven-dried Rhodospirillum rubrum consumption and serum LDL cholesterol concentrations in slightly hypercholesterolemic men. A further objective was to examine the effects of oven-dried Rhodospirillum rubrum consumption on additional CVD risk markers like circulating concentrations of total cholesterol, triacylglycerol (TAG), HDL cholesterol, glucose and hsCRP as well as systolic (SBP) and diastolic blood pressure (DBP) and resting heart rate in slightly hypercholesterolemic men. In addition, an objective was to evaluate the effects of oven-dried Rhodospirillum rubrum consumption on markers for liver function (ALT, AST, γGT), and kidney (creatinine) function as well as heart function (NTProBNP, vWF, c-Troponin T) in slightly hypercholesterolemic men.

### Study Design

A 4-weeks randomized, double-blind placebo-controlled dose response trial with a parallel design was carried out in slightly hypercholesterolemic men.

### Screening

Before screening, men were informed about the procedures in the study and informed consent was obtained. After the screening visit, study participants that fulfilled all inclusion criteria were asked to participate. Men were informed about their screening results, including anthropometric measures (weight, length, body mass index), SBP and DBP, resting heart rate, serum total cholesterol and triacylglycerol concentrations. Furthermore, liver (ALT, AST, γGT) and kidney (creatinine) function were determined. When treatment with drugs or lifestyle interventions was advised according to the Standard for cardiovascular risk management of the Dutch general practitioners community (NHG), study subjects were advised to consult their general practitioner. The screening resulted in a final count of eighty-two men to participate in the trial.

### Test Days

On the days preceding blood sampling, men were asked not to perform any strenuous physical exercise or to consume alcohol. On the morning of blood sampling—after a 12 hr overnight fast (from 8.00 PM)—men were only allowed to drink a glass of water. Men were also asked not to change their habitual diet prior to the test days. Finally, men were asked to come to the Metabolic Research Unit Maastricht (MRUM, The Netherlands) by public transport or car (and not by foot or bike) to standardize measurements as much as possible. All men were randomly allocated to one of the four treatment arms. In this way variation due to drift of variables with time was eliminated. The total study took 8 weeks, including a run-in period of 2 weeks, an experimental period of 4 weeks and a post study washout period of 2 weeks. Men were always arriving in the morning at the MRUM test facilities always in fasting condition. All test days started with anthropometric measurements (weight, length, waist and hip circumferences). Then, SBP, DBP and resting heart rate was determined. Finally, a blood sample was taken to measure a series of CVD risk markers as well as markers for liver, kidney and heart function.

### Intervention

The experimental design of the study is shown in FIG. 9. The study had a randomized-controlled, double-blind, placebo-controlled parallel design with a run-in period of 2 weeks, an experimental period of 4 weeks, and a wash-out period of 2 weeks. During the run-in period, all men received placebo capsules to become familiarized with study procedures. A 4 weeks experimental period was long enough to reach a new steady state in serum lipid and lipoprotein concentrations. The wash-out period—men were again provided with placebo capsules like in the run-in period—was used to check if post-treatment serum lipid and lipoprotein concentrations have returned to pre-treatment values after discontinuation of the intervention. Four study groups of 20 men each were included. One group served as control and the other three groups received increasing doses of oven-dried Rhodospirillum rubrum (0.25, 0.5 and 1.0 g/day). Therefore this study provided information (i) whether oven-dried Rhodospirillum rubrum (oven-dried R. rubrum cells obtained with refractive drying at 60° C.) lowers serum LDL cholesterol concentrations in slightly hypercholesterolemic men and (ii) whether dose-response relationship exists. During the run in period of 2 weeks as well as the post study wash out period of two weeks all men received placebo capsules containing the inert filling material microcrystalline cellulose. During the intervention period of 4 weeks, men received daily experimental capsules either containing the same inert filling material microcrystalline cellulose (control group) or capsules containing the oven-dried Rhodospirillum rubrum. The men were randomly allocated to one of the four arms. Both researchers and volunteers were blinded, since the test products were distributed in a closed bottle only with a participant number. The content of the bottle with capsules could only be found in a table where the participant numbers were connected to the type of study product. This file with the study code was only broken at the end of the study after all the (statistical) analyses were completed.

The primary outcome parameter is a change in serum LDL cholesterol concentrations. For this, blood was sampled once at the beginning of the run in period (day 0), twice at the end of the run in period/start of the experimental period (days 11 and 14), four times during the experimental period (days 21, 28, 39 and 42), and twice at the end of the post study wash out period (days 49 and 56). Since this trial was the first proof of principle study in humans, the blood samples were analyzed weekly for a panel of “safety” parameters like markers for liver function (ALT, AST, γGT), and kidney function (creatinine) as well as heart function (NT-ProBNP, vWF, c-Troponin T). The study was carried out under GCP. Several previous studies with cholesterol-lowering compounds, such as plant sterol and stanols, have reported that serum LDL cholesterol concentrations can be reduced within 4 weeks of consumption. This cholesterol-lowering effect has been shown in several population groups, including slightly hypercholesterolemic subjects.

### Study Population

### Population

Eighty-two (N=82) healthy men with a slightly elevated fasting serum total cholesterol concentration (between 5.0-8.0 mmol/1) were recruited. Seventy-six (N=76) men completed the study.

### Screening Visit

The men were invited for a screening visit, this visit included recording of:
Body weight
Height
Blood pressure: diastolic and systolic
Serum total cholesterol and triacylglycerol concentrations
Serum ALT, AST, γGT and creatinine concentrations
Use of medication
(at that time) Current and history of diseases

All men attended the metabolic research facilities (MRUM) for a screening visit. During this visit, anthropometric measurements (weight, length, waist and hip circumference, body mass index) were performed and blood pressure (BP) (SBP and DBP) was determined in four-fold (the first measurement was discarded and the last three measurements were averaged). In addition, a venous blood sample (5.0 mL) was drawn for analysis of serum total cholesterol, triacylglycerol, ALT, AST, γGT and creatinine concentrations In case the study participants were blood donor, they were told that they cannot donate blood from 8 weeks before the start of the study, during the study and for 4 weeks after completion of the study. When the screened men fulfilled all inclusion criteria, they were allowed to enter the study.

### Inclusion Criteria

The inclusion criteria were:
Aged between 18-75 years;
Men;
Minimum 80 kg body weight;
Serum total cholesterol between 5.0-8.0 mmol/L (further testing was recommended for excessive hyperlipidemia [serum total cholesterol 8.0 mmol/L] according to the Standard for cardiovascular risk management of the Dutch general practitioners community [NHG]);
Serum triacylglycerol concentrations <4.5 mmol/L;
No signs of liver and/or kidney dysfunction;
No diabetic patients;
No familial hypercholesterolemia;
No abuse of drugs;
Not more than 4 alcoholic consumptions per day with a maximum of 21 per week;
Stable body weight (weight gain or loss <3 kg in the past three months);
No use of medication known to treat blood pressure, lipid or glucose metabolism;
No use of an investigational product within another biomedical intervention trial within the previous 1-month;
No severe medical conditions that could interfere with the study, such as epilepsy, asthma, kidney failure or renal insufficiency, chronic obstructive pulmonary disease, inflammatory bowel diseases, auto inflammatory diseases and rheumatoid arthritis;
No active cardiovascular disease like congestive heart failure or cardiovascular event, such as an acute myocardial infarction or cerebrovascular accident;
Willingness to give up being a blood donor from 8 weeks before the start of the study, during the study and for 4 weeks after completion of the study;
No difficult venipuncture as evidenced during the screening visit;
Willing to comply to study protocol during study;
Informed consent signed.

### Exclusion Criteria

The exclusion criteria were:
Serum total cholesterol <5.0 mmol/L or ≥8.0 mmol/L;
Serum triacylglycerol concentrations 4.5 mmol/L;
Signs of liver and/or kidney dysfunction;
Diabetic patients;
Familial hypercholesterolemia;
Abuse of drugs;
More than 4 alcoholic consumptions per day or 21 per week;
Unstable body weight (weight gain or loss >3 kg in the past three months);
Use of medication known to treat blood pressure, lipid or glucose metabolism;
Use of an investigational product within another biomedical intervention trial within the previous 1-month;
Severe medical conditions that could interfere with the study, such as epilepsy, asthma, kidney failure or renal insufficiency, chronic obstructive pulmonary disease, inflammatory bowel diseases, auto inflammatory diseases and rheumatoid arthritis;
Active cardiovascular disease like congestive heart failure or cardiovascular event, such as an acute myocardial infarction or cerebrovascular accident;
Not willing to give up being a blood donor from 8 weeks before the start of the study, during the study or for 4 weeks after completion of the study;
Not or difficult to venipuncture as evidenced during the screening visit;
Use of over-the-counter and prescribed medication or supplements, which may interfere with study measurements to be judged by the principal investigator;
Use of oral antibiotics in 40 days or less prior to the start of the study;
Blood donation in the past 3 months before the start of the study;
Not willing to comply to study protocol during study or sign informed consent.

### Sample Size Calculation

The primary outcome parameter of this study was serum LDL cholesterol. The sample size calculations were based on the following criteria: a significance level alpha of 0.05 (2-sided), a power of 0.80, an allocation ratio of 1/3, an average expected reduction of 0.41 mmol/L (10%) in serum LDL-C in the three groups that receive the oven-dried Rhodospirillum rubrum material, and an estimated within-subject SD of 0.45 mmol/L. Since the effect of the three groups that receive oven-dried Rhodospirillum rubrum is averaged, it is expected that the within-subject SD in the interventions groups will be 0.65 mmol/L instead of 0.45 mmol/L. Based on these criteria, the three intervention groups should include 60 subjects and the control group 20 subjects (using G-power). Therefore the study was conducted with four groups of 20 men each completing the study.

### Treatment of Subjects

### Randomization, Blinding and Treatment Allocation

The men were randomly allocated to one of the four experimental conditions, based upon a computer-generated table with random numbers. For this, a categorical list in logical order was created by an independent person. Subjects from the same household were allocated to the same treatment. The randomization code was only then broken after all analyses were completed.

### Investigational Products

The capsules used in this intervention were produced by Ambi Pack BV in Hendrik-Ido-Ambacht (The Netherlands) according to GCP principles. Ambi Pack BV is certified to produce capsules for research purposes. The material used to fill the capsules, i.e. the inert placebo material microcrystalline cellulose (MCC) or the oven-dried Rhodospirillum rubrum material in the indicated amounts is safe for human consumption. The MCC as well as the gelatin capsules were provided by Ambi Pack BV. The placebo capsules contained 250 mg MCC. The oven-dried Rhodospirillum rubrum material was produced by Algenkwekerij AW Van Bennekom, Schalkwijk (The Netherlands) (See Example 7 here above) in sterile productions lines. Rhodospirillum rubrum material may also be produced by AlgoSolis R&D (Saint-Nazaire, France) by air drying the Rhodospirillum rubrum cells at a temperature of 60° C. to obtain biomass. The oven-dried biomass is then packed in capsules by ProPhar Laboratories (Angers, France). Capsules were always packed in week portions and subjects were asked to store the capsules in the fridge (+4° C.). Capsules were first provided in two bottles with 28 capsules each covering the run-in period (14 days) and were labelled to use 2 capsules during lunch and 2 capsules during dinner (4 capsules/day). Next men got new bottles of capsules for the experimental day (28 days) with specific instructions. The first group got the instruction to use daily 4 white capsules (placebo capsules), the second group 4 blue capsules (R. rubrum capsules, 1.00 g-group), the third group 2 white and 2 blue (0.50 g-group) and the last group 3 white and 1 blue capsules (0.25 g-group). Again, the subjects had the instructions to take 2 capsules at lunch and 2 capsules at dinner. The 0.50 g-group was instructed to take 1 blue and 1 white capsule at lunch and dinner. The 0.25 g-group was instructed to take 2 white capsules at lunch and 1 blue and 1 white capsule at dinner. Finally they got again two bottles of 28 capsules covering the post study wash out period identical to those in the run-in period. Capsules and instructions were distributed in white jars to blind investigators. In every bottle there were several capsules as surplus to be able to actively monitor real intake of the capsules by counting back the amount of capsules returned at the end of the three different periods. Further characteristics of the oven-dried Rhodospirillum rubrum material (oven-dried R. rubrum cells obtained with refractive drying at 60° C.) are detailed here above in Example 7.

Analyzing the previously conducted animal studies (mice, rates), effects on LDL-cholesterol seemed to be dose dependent (up to 40-50% reduction) and appeared to be linked to reduced intestinal cholesterol absorption. Effects were present when using the lyophilized intact organism (Example 1-4), and also when a petroleum-ether fraction ‘l.1’ of freshly cultured cells (Example 5) were used by the inventors. In these animals, there were no side effects observed.

Based on the doses (10% w/w) used in the previous rodent studies applying freeze-dried cells, a cell fraction and used in the mouse study applying oven-dried R. rubrum cells (oven-dried R. rubrum cells obtained with refractive drying at 60° C.) for establishing the repetitive LDL-cholesterol lowering activity of a new batch of now oven-dried R. rubrum cells (Examples 6, 7), the following rationale to translate these concentrations to the human situation was followed. Calculations were based on the body weight of (young) rats. When converting the concentrations of Rhodospirillum rubrum (10%) in the food of young rats to a daily dose for a human subject, the conversion factor of 0.12 for subacute studies (EFSA documents) was used.

### Dosage in Rats:

10% w/w Rhodospirillum rubrum is equal to 100.000 mg/kg in feed; and 100.000 mg/kg*0.12=12.000 mg/kg body weight=12 g/kg body weight. Since only one dose was tested, the 12 g/kg is considered as the lowest observed adverse effect levels (LOAEL). A no observed adverse effect level (NOAEL) was obtained by dividing the dosage by 10: 1.2 g/kg body weight. To convert this dosage into a human equivalent dose, the EFSA Scientific Committee proposes to use an uncertainty factor of 100 when chemical-specific data on kinetics is missing: 10 for inter-species variability and 10 for intra-human variability.

### Dosage in Humans:

1.2 g/kg body weight in young rats (NOAEL)/100=0.0012 g/kg body weight; and 0.0012 g/kg body weight=12 mg/kg body weight.

For an 80 kg man, the dosage of 12 mg/kg body weight would amount to 0.96 gram per day. Therefore, the highest dose levels tested in the current study protocol for the DBRCCT was 1.0 gram per day. This is also why a minimal bodyweight of 80 kg was one of the inclusion criteria for the study in this study where the effects of 0.25, 0.5 and 1.0 gram/day were evaluated compared to placebo. The rationale for the dosages was based on EFSA documents. Subjects were provided with capsules containing the oven-dried, dead biomass of R. rubrum cell culture. In addition, next to a general panel of safety markers (liver function, kidney function, vWF and hsCRP) also a number of additional parameters were measured to monitor potential effects on the heart like N-terminal fragment B-type natriuretic peptide (NT-ProBNP) and c-Troponin T.

### Methods

### Procedures During the Screening

Men attended the metabolic research facilities (MRUM) for a screening visit. During the screening visit, anthropometric measurements (weight, length, body mass index) were performed and BP (SBP and DBP) and resting heart rate were determined in four-fold (the first measurement was discarded and the last three measurements were averaged). In addition, a venous blood sample (5.0 mL) was drawn for analysis of serum total cholesterol, HDL cholesterol, triacylglycerol, ALT, AST, γGT and creatinine concentrations.

### Blood Sampling During the Study

As depicted in the experimental design (FIG. 9), fasting blood was sampled once at the beginning of the run-in period (day 0), twice at the end of the run in period (days 11 and 14), four times during the experimental period (days 21, 28, 39 and 42), and twice at the end of the post study wash out period (days 49 and 56). Fasting blood was collected for the analysis of a variety of biochemical parameters related to lipid, lipoprotein and glucose metabolism. All venipunctures and other measurements were performed as much as possible by the same investigator, at the same location, and at the same time of the day. Serum or plasma was obtained by low-speed centrifugation and stored as appropriate. Samples from one subject were analyzed within one run at the end of the study under strict quality control. The amount of blood drawn was 230 mL in total during the entire study (one time 5.0 mL during the screening visit, 9×25 ml during the study).

### Analysis in Fasting Serum Samples and Plasma Samples

Serum TC, HDL-C and TAG concentrations were measured in all samples taken during the study and LDL-C concentrations were calculated
Plasma (NaF) glucose concentrations were measured in all samples taken during the study
Parameters reflecting liver and kidney function (ALT, AST, γGT and creatinine) as well as NT-ProBNP, vWF, c-Troponin T and hsCRP concentrations were measured in serum in all samples taken during the study as a measure of general “safety”. ALT, AST, γGT and creatinine were assessed by spectrophotometric assays (Cobas 8000, Roche diagnostic System). c-Troponin T and NT-ProBNP were assessed by immunoassays (Cobas 8000, Roche Diagnostic System). vWF was measured using ELISA (Sigma-Aldrich, Darmstadt, Germany). hsCRP was assessed by immunoturbidimetric assay (Horiba ABX). Systolic and diastolic blood pressure and heart rate were measured using an Omron M7 (Omron Healthcare Co. Ltd., Kyoto, Japan).

All analyses related to lipid, lipoprotein and glucose metabolism were done at the end of the study. However, since this was the first proof of principle study in humans, the blood samples were analyzed for a panel of “safety” parameters like liver function, kidney function, NT-ProBNP, vWF, c-Troponin T and hsCRP immediately after sampling. Safety data was monitored by a blinded, independent physician at several pre-determined time points during the trial.

### Anthropometric Measurements

At each visit, anthropometric measurements (weight, length, waist and hip circumferences) without shoes and heavy clothing were measured.

### Blood Pressure

Blood pressure (SBP and DBP) was determined in four-fold (the first measurement was discarded and the last three measurements were averaged) during every visit.

### Resting Heart Rate

Resting heart rate was measured during every visit after a 5-minute rest in a seated position. Measurements were done in four-fold and the last three measurements were always averaged for data analyses. AEs, SAEs

### Adverse Events (AEs)

Adverse events are defined as any undesirable experience occurring to a subject during the study, whether or not considered related to the investigational product. All adverse events reported spontaneously by the subject or observed by the investigator or his staff, if any, would have been recorded. Remark: no adverse events, serious adverse events, complaints, health issues, etc., etc., were reported by any of the 80 healthy human subjects who started and finalized the clinical trial.

### Serious Adverse Events (SAEs)

A serious adverse event is any untoward medical occurrence or effect that
results in death;
is life threatening (at the time of the event);
requires hospitalisation or prolongation of existing inpatients' hospitalisation;
results in persistent or significant disability or incapacity;
is a congenital anomaly or birth defect; or
any other important medical event that did not result in any of the outcomes listed above due to medical or surgical intervention but could have been based upon appropriate judgement by the investigator.

No adverse events were reported in relation to the use of the Rhodospirillum rubrum

### Statistical Analysis

### Descriptive Statistics

For normal distributed parameters, data are presented as mean values plus standard deviation. Non-normal distributed parameters are presented as median (ranges).

### Statistical Analysis

The data is visualized and checked for biological plausibility or potential technical difficulties. It is determined whether a normal distribution is valid or that transformation is required using histograms and appropriate normality checks. For normal distributed parameters, data is presented as mean values plus standard deviation. Non-normal distributed parameters are presented as median (ranges). Values at the start of the intervention and at the end of the intervention period were averaged before statistical comparisons were made. The average effect in the three intervention groups is compared with the effect in the control group (placebo) using an unpaired T-test. Furthermore, to test for a dose-response effect as a secondary objective, the changes in each intervention group (except for the placebo group) were compared with the changes from the previous dosage using univariate analysis of variance (ANOVA). Differences in baseline characteristics between control and Rhodospirillum rubrum groups is tested using univariate analysis of variance (ANOVA). In case of significant differences in baseline characteristics, these characteristics would have been included in the analysis as covariates. Non-parametric tests were used where appropriate. Differences were considered to be statistically significant when P<0.05. Statistical analyses were performed using SPSS 25.0 for Mac (SPSS Incorporated, Chicago, IL, USA). Missing data were not replaced by estimates. Men that dropped-out were excluded from the statistical analyses.

### Results of the First-In-Human Clinical Trial Phase II

In total 76 men completed the trial. Data of 6 further subjects was removed from statistical analysis due to excessive weight change (N=3), metabolic instability (N=1), smoking during the trial (N=1) and elevated TC concentrations throughout the whole trial (N=1). Thus, the data of 70 subjects was included for statistical analysis. In summary, the men in the investigational product treatment groups showed a total cholesterol value that decreased 0.15-0.17 mmol/L during the course of the study. The decrease in total cholesterol was solely attributed to a decrease in the level of LDL-cholesterol. Proof that the R. rubrum cells do not affect HDL-cholesterol while lowering LDL-cholesterol can also be found in the total cholesterol/HDL-cholesterol ratio. In Table 2, the total cholesterol/HDL-cholesterol ratio (TC/HDL ratio) can be found for all groups. As can be seen in this Table, the experimental groups had a lower TC/HDL ratio after 4 weeks, while the placebo group had a higher TC/HDL ratio. This means that the R. rubrum cells are capable of lowering the LDL-fraction of the total cholesterol, while HDL is either unaffected or increased. The placebo group did not show a decrease (neither an increase) in LDL-cholesterol levels. For the men treated with refractively dried R. rubrum cells the LDL-cholesterol concentration was 2%-6% lower at the end of the four-weeks treatment period compared to the LDL-cholesterol level at the start of the clinical trial. For all groups the HDL-cholesterol concentration remained essentially constant during the course of the clinical trial. Thus, daily intake of R. rubrum cells does not influence blood levels of HDL-cholesterol. Comparing the groups of human subjects who received 0.25 gram R. rubrum cells per day (0.25 gram once daily), 0.50 gram R. rubrum cells per day (0.25 gram twice daily) or 1.0 gram R. rubrum cells per day (0.50 gram twice daily), the highest dose resulted in the highest decrease in LDL-cholesterol concentration during the course of the treatment period. A dose response effect was apparent when the effects of the three doses on LDL-cholesterol concentration were considered. For each of the three treatment groups, a linear decrease of the LDL-cholesterol (and total cholesterol) concentration is determined during the four-weeks treatment period. Thus, a linear relationship between percentage change in total cholesterol and the dose of Rhodospirillum rubrum is indicated. The concentration of non-cholesterol sterols campesterol and sitosterol in the blood of the three groups that received a dose of R. rubrum cells, decreased during the course of the four-weeks treatment period. Furthermore, a dose response effect was apparent: the group treated with 0.25 gram R. rubrum cells showed a lower decrease in blood levels of the two non-cholesterol sterols than the groups treated with the two higher doses, and the group treated with 1.0 gram R. rubrum cells showed the largest decrease in blood levels of campesterol and sitosterol. Blood levels and changes in blood levels of these two non-cholesterol sterols is a marker for absorption of cholesterol out of the intestine into the blood circulation. Herewith, it is confirmed that the LDL-cholesterol lowering effect of treating healthy human subjects with oven-dried R. rubrum cells is established by an inhibitory activity of the R. rubrum cells on uptake of cholesterol from the intestine into the blood circulation. Said cholesterol in the intestine originates from the diet ingested by the clinical trial participants and originates from the biliary cholesterol.

The inventors established that the plasma LDL-cholesterol lowering activity of R. rubrum cells is condensed in the petroleum-ether fraction l.1 (Example 5), comprising a series of xantophylls (carotenoids rhodovibrin, 1-hydroxyspirilloxanthin, 3,4-didehydro-rhodopin, chloroxanthin, rhodopin, spirilloxanthin, and 3,4-dihydro-spirilloxanthin), as well as the (ubi)quinones and (ubi)quinoles ubiquinol-10, ubiquinone-9, ubiquinone-10, and rhodoquinone-10, as well as the bacteriopheophytins a, geranylgeranyl bacteriopheophytin a, and phytyl derivative of bacteriopheophytin a. This fraction l.1 derived from freshly cultured R. rubrum cells contributes 1.5% to the total mass of the R. rubrum cells (on a dry matter basis) from which the petroleum-ether fraction is derived. Therefore, about 1.5% R. rubrum fraction based on the total dry weight of R. rubrum cells, comprises the active fraction when plasma LDL-cholesterol lowering activity is considered. Correlating these amounts with the current clinical trial, the groups treated with oven-dried whole R. rubrum cells thus received 1.5% of 0.25 gram, 0.50 gram, 1.0 gram active compound(s) at maximum: 3.75 mg, 7.5 mg, 15 mg active compound(s), respectively. Most likely, since fraction l.1 may comprise further, inactive compounds, the true dose of active compound(s) is lower. These active compound(s) comprise one or more of the identified carotenoids, and/or one or more of the (ubi)quinones/(ubi)quinols, and/or one or both of the bacteriopheophytins a. For comparison, daily consumption by humans of 3 grams phytosterols results in a decrease of total cholesterol in plasma of 8%-10%. Therefore, the effect established with for example the highest current dose of R. rubrum cells (15 mg at maximum in the 1.0 gram) is exerted with an about 200-fold lower dose of active compound(s), on a mass basis.

Comparing the final reduced plasma LDL-cholesterol levels with the three doses of R. rubrum cells, a linear dose response is observed. Therefore, doses higher than 1.0 gram R. rubrum cells daily will highly likely result in even lower LDL-cholesterol levels compared to the concentration at the start of daily intake of the R. rubrum cells.

Anthropometric measurements were unaffected by the intake of R. rubrum cells. Changes in weight were 0.0±0.7 kg in the placebo group, −0.1±0.9 kg in the 0.25 gram group, 0.0±0.8 kg in the 0.50 gram group and −0.4±0.5 kg in the 1.00 gram group. In addition, changes in waist circumference and the waist-to-hip circumference ratio also did not differ significantly between the groups. Thus, intake of R. rubrum cells does not negatively affect the anthropometrics of the subjects.

Fortunately, not a single adverse effect, health issue, complaint, etc., etc., that could perhaps related to or attributed to the daily intake of R. rubrum cells have been reported by any of the 60 men in the three treatment groups receiving R. rubrum cells (the same for the placebo group: zero adverse events reported and observed). Tables 1, 2, 3 and 4 display that no significant changes in the measured health-related parameters AST, ALT, γGT, creatinine, NT-ProBNP, vWF, c-Troponin T, plasma glucose, hsCRP, systolic blood pressure (SBP) and diastolic blood pressure (DBP), and resting heart rate were observed. Most importantly and surprisingly, none of the parameters varied to such a degree that they were outside the range which is considered “normal” for a healthy individual. Therefore, it is included that daily intake of R. rubrum cells provides for a convenient and safe treatment regimen.

TABLE 1
concentrations of the safety parameters measured
in blood serum of human subjects at baseline and after
4 weeks of placebo and Rhodospirillum rubrum consumption

| | Placebo | 0.25 grams | 0.50 grams | 1.0 grams |
|---|---|---|---|---|
| | (N = 17) | (N = 18) | (N = 18) | (N = 17) |
| AST (U/L) | | | | |
| Baseline | 26.9 ± 6.4 | 25.8 ± 4.8 | 24.3 ± 5.4 | 27.9 ± 6.6 |
| 4 weeks | 24.8 ± 6.5 | 26.1 ± 5.3 | 26.3 ± 8.5 | 27.1 ± 7.4 |
| 4-wk change | −2.1 ± 4.9 | 0.3 ± 4.7 | 2.1 ± 8.1 | −0.8 ± 5.4 |
| ALT (U/L) | | | | |
| Baseline | 31.2 ± 11.6 | 30.2 ± 11.4 | 26.7 ± 7.4 | 30.9 ± 9.5 |
| 4 weeks | 27.4 ± 11.1 | 30.6 ± 12.2 | 27.2 ± 8.1 | 30.5 ± 8.9 |
| 4-wk change | −3.8 ± 6.0 | 0.4 ± 6.9 | 0.6 ± 5.8 | −0.4 ± 5.4 |
| □GT (U/L) | | | | |
| Baseline | 28.4 ± 7.9 | 35.5 ± 24.2 | 29.4 ± 13.7 | 33.3 ± 20.7 |
| 4 weeks | 27.5 ± 8.3 | 35.1 ± 22.1 | 30.3 ± 15.7 | 32.0 ± 19.7 |
| 4-wk change | −0.9 ± 4.5 | −0.4 ± 5.7 | 0.9 ± 6.6 | −1.3 ± 4.1 |
| Creatinine (μmol/L) | | | | |
| Baseline | 89.6 ± 10.5 | 95.0 ± 18.1 | 88.2 ± 10.7 | 92.2 ± 10.8 |
| 4 weeks | 88.8 ± 10.5 | 94.4 ± 15.2 | 90.8 ± 9.6 | 92.9 ± 12.7 |
| 4-wk change | −0.8 ± 7.6 | −0.6 ± 10.2 | 2.7 ± 7.5 | 0.6 ± 6.0 |
| NT-ProBNP (pmol/L) | | | | |
| Baseline | 5.7 ± 3.3 | 5.2 ± 2.1 | 7.4 ± 8.0 | 7.0 ± 6.2 |
| 4 weeks | 6.1 ± 3.9 | 5.6 ± 3.0 | 6.1 ± 3.8 | 7.3 ± 6.5 |
| 4-wk change | 0.4 ± 2.7 | 0.3 ± 2.3 | −1.3 ± 5.9 | 0.3 ± 4.3 |
| vWF (μg/mL) | | | | |
| Baseline | 22.1 ± 13.2 | 18.3 ± 11.2 | 18.6 ± 12.8 | 21.8 ± 11.2 |
| 4 weeks | 22.9 ± 13.5 | 23.6 ± 19.6 | 19.6 ± 13.0 | 25.0 ± 15.9 |
| 4-wk change | 0.8 ± 13.3 | 5.3 ± 13.8 | 0.9 ± 7.7 | 3.1 ± 12.4 |
| Troponin T (ng/L) | | | | |
| Baseline | 9.4 ± 3.2 | 9.4 ± 3.7 | 7.5 ± 2.8 | 10.4 ± 7.5 |
| 4 weeks | 8.7 ± 2.9 | 9.8 ± 4.5 | 7.9 ± 2.2 | 10.2 ± 7.8 |
| 4-wk change | −0.7 ± 1.5 | 0.4 ± 1.7 | 0.4 ± 1.9 | −0.2 ± 2.6 |
| hsCRP (mg/L) | | | | |
| Baseline | 3.0 ± 3.0 | 1.2 ± 0.2 | 1.5 ± 1.4 | 2.1 ± 0.4 |
| 4 weeks | 3.5 ± 5.0 | 1.7 ± 0.1 | 2.5 ± 3.0 | 1.7 ± 0.3 |

Values are means ± SD

TABLE 2
Total, LDL and HDL cholesterol, triacglycerol and
TC/HDL ratio at baseline and 4 weeks
after Rhodopspirrilum rubrum consumption

| | Placebo | 0.25 grams | 0.50 grams | 1.0 grams |
|---|---|---|---|---|
| | (N = 17) | (N = 18) | (N = 18) | (N = 17) |
| Total | | | | |
| cholesterol | | | | |
| (mmol/L) | | | | |
| Baseline | 5.67 ± 0.73 | 5.78 ± 0.56 | 6.26 ± 0.72 | 5.89 ± 0.71 |
| 4 weeks | 5.80 ± 0.58 | 5.74 ± 0.78 | 6.13 ± 0.63 | 5.75 ± 0.70 |
| 4-wk change | 0.12 ± 0.59 | −0.04 ± 0.40 | −0.13 ± 0.52 | −0.14 ± 0.39 |
| LDL | | | | |
| cholesterol | | | | |
| (mmol/L) | | | | |
| Baseline | 3.89 ± 0.70 | 3.82 ± 0.54 | 4.18 ± 0.78 | 3.95 ± 0.77 |
| 4 weeks | 3.93 ± 0.69 | 3.77 ± 0.71 | 4.07 ± 0.74 | 3.85 ± 0.82 |
| 4-wk change | 0.04 ± 0.49 | −0.05 ± 0.32 | −0.11 ± 0.46 | −0.10 ± 0.31 |
| HDL | | | | |
| cholesterol | | | | |
| (mmol/L) | | | | |
| Baseline | 1.20 ± 0.18 | 1.22 ± 0.26 | 1.36 ± 0.40 | 1.29 ± 0.33 |
| 4 weeks | 1.22 ± 0.21 | 1.24 ± 0.26 | 1.37 ± 0.43 | 1.25 ± 0.29 |
| 4-wk change | 0.03 ± 0.13 | 0.02 ± 0.06 | 0.01 ± 0.06 | −0.04 ± 0.13 |
| Triacylglycerol | | | | |
| (mmol/L) | | | | |
| Baseline | 1.39 ± 0.31 | 1.63 ± 0.82 | 1.29 ± 0.52 | 1.51 ± 0.79 |
| 4 weeks | 1.45 ± 0.43 | 1.68 ± 0.92 | 1.35 ± 0.67 | 1.48 ± 0.78 |
| 4-wk change | 0.06 ± 0.33 | 0.05 ± 0.23 | 0.06 ± 0.27 | −0.03 ± 0.28 |
| TC/HDL ratio | | | | |
| Baseline | 4.90 ± 1.06 | 4.90 ± 0.93 | 4.99 ± 1.79 | 4.94 ± 1.22 |
| 4 weeks | 4.93 ± 1.07 | 4.79 ± 0.89 | 4.84 ± 1.53 | 4.94 ± 1.18 |

Values are means ± SD

TABLE 3
Safety parameters measured at baseline
and after 1, 2 and 4 weeks of intervention

| | Placebo | 0.25 grams | 0.50 grams | 1.0 grams |
|---|---|---|---|---|
| | (N = 17) | (N = 18) | (N = 18) | (N = 17) |
| AST (U/L) | | | | |
| Baseline | 26.9 ± 6.4 | 25.8 ± 4.8 | 24.3 ± 5.4 | 27.9 ± 6.6 |
| 1 week | 26.2 ± 5.4 | 25.9 ± 5.2 | 23.6 ± 5.4 | 26.9 ± 7.3 |
| 2 weeks | 25.2 ± 5.8 | 25.3 ± 6.2 | 23.4 ± 5.0 | 25.9 ± 5.9 |
| 4 weeks | 24.8 ± 6.5 | 26.1 ± 5.3 | 26.3 ± 8.5 | 27.1 ± 7.4 |
| ALT (U/L) | | | | |
| Baseline | 31.2 ± 11.6 | 30.2 ± 11.4 | 26.7 ± 7.4 | 30.9 ± 9.5 |
| 1 week | 28.2 ± 9.1 | 27.7 ± 9.8 | 25.9 ± 6.5 | 30.7 ± 7.5 |
| 2 weeks | 28.1 ± 9.4 | 28.8 ± 12.3 | 25.9 ± 7.7 | 29.2 ± 6.8 |
| 4 weeks | 27.4 ± 11.1 | 30.6 ± 12.2 | 27.2 ± 8.1 | 30.5 ± 8.9 |
| □GT (U/L) | | | | |
| Baseline | 28.4 ± 7.9 | 35.5 ± 24.2 | 29.4 ± 13.7 | 33.3 ± 20.7 |
| 1 week | 27.2 ± 8.0 | 33.6 ± 21.4 | 29.8 ± 13.7 | 32.1 ± 20.1 |
| 2 weeks | 27.2 ± 7.0 | 33.6 ± 21.5 | 30.0 ± 13.4 | 31.4 ± 19.0 |
| 4 weeks | 27.5 ± 8.3 | 35.1 ± 22.1 | 30.3 ± 15.7 | 32.0 ± 19.7 |
| Creatinine | | | | |
| (μmol/L) | | | | |
| Baseline | 89.6 ± 10.5 | 95.0 ± 18.1 | 88.2 ± 10.7 | 92.2 ± 10.8 |
| 1 week | 89.2 ± 9.9 | 94.9 ± 14.8 | 90.2 ± 9.9 | 94.2 ± 11.4 |
| 2 weeks | 89.9 ± 13.5 | 94.8 ± 13.3 | 93.6 ± 10.5 | 94.2 ± 12.2 |
| 4 weeks | 88.8 ± 10.5 | 94.4 ± 15.2 | 90.8 ± 9.6 | 92.9 ± 12.7 |
| NT-ProBNP | | | | |
| (pmol/L) | | | | |
| Baseline | 5.7 ± 3.3 | 5.2 ± 2.1 | 7.4 ± 8.0 | 7.0 ± 6.2 |
| 1 week | 6.0 ± 3.6 | 5.6 ± 3.1 | 6.0 ± 4.2 | 8.5 ± 7.6 |
| 2 weeks | 6.5 ± 4.2 | 6.3 ± 3.4 | 6.3 ± 4.4 | 7.4 ± 7.4 |
| 4 weeks | 6.1 ± 3.9 | 5.6 ± 3.0 | 6.1 ± 3.8 | 7.3 ± 6.5 |
| vWF (μg/mL) | | | | |
| Baseline | 22.1 ± 13.2 | 18.3 ± 11.2 | 18.6 ± 12.8 | 21.8 ± 11.2 |
| 1 week | 20.7 ± 11.3 | 14.7 ± 10.7 | 16.2 ± 12.0 | 21.2 ± 10.8 |
| 2 weeks | 22.6 ± 10.6 | 17.1 ± 10.5 | 18.8 ± 10.3 | 21.2 ± 13.3 |
| 4 weeks | 22.9 ± 13.5 | 23.6 ± 19.6 | 19.6 ± 13.0 | 25.0 ± 15.9 |
| Troponin | | | | |
| T (ng/L) | | | | |
| Baseline | 9.4 ± 3.2 | 9.4 ± 3.7 | 7.5 ± 2.8 | 10.4 ± 7.5 |
| 1 week | 8.8 ± 3.0 | 9.6 ± 4.4 | 7.9 ± 2.9 | 9.7 ± 6.5 |
| 2 weeks | 8.8 ± 2.9 | 9.8 ± 4.0 | 8.4 ± 3.6 | 10.1 ± 7.3 |
| 4 weeks | 8.7 ± 2.9 | 9.8 ± 4.5 | 7.9 ± 2.2 | 10.2 ± 7.8 |

Values are means ± SD

TABLE 4
Plasma glucose concentrations and blood pressure at baseline,
after 1, 2 and weeks of intervention, and after the run-out period.
Serum hsCRP concentrations at baseline and after 1, 2 and 4 weeks
of intervention are depicted. Resting heart rate (beats per minute)
for baseline and after 4 weeks of intervention are depicted.

| | Placebo | 0.25 grams | 0.50 grams | 1.0 grams |
|---|---|---|---|---|
| | (N =17) | (N = 18) | (N =18) | (N =17) |
| Glucose | | | | |
| (mmol/L) | | | | |
| Baseline | 5.71 ± 0.46 | 5.64 ± 0.57 | 5.65 ± 0.50 | 5.88 ± 0.63 |
| run-in | | | | |
| Baseline | 5.59 ± 0.47 | 5.65 ± 0.53 | 5.62 ± 0.41 | 5.78 ± 0.62 |
| intervention | | | | |
| 1 week | 5.58 ± 0.47 | 5.61 ± 0.59 | 5.65 ± 0.48 | 5.72 ± 0.58 |
| 2 weeks | 5.65 ± 0.51 | 5.58 ± 0.49 | 5.72 ± 0.47 | 5.67 ± 0.51 |
| 4 weeks | 5.64 ± 0.40 | 5.64 ± 0.51 | 5.69 ± 0.47 | 5.77 ± 0.60 |
| Run-out | 5.72 ± 0.47 | 5.66 ± 0.49 | 5.65 ± 0.49 | 5.78 ± 0.59 |
| hsCRP | | | | |
| (mg/L) | | | | |
| Baseline | 1.5 (0.3-10.4) | 1.2 (0.2-2.7) | 1.0 (0.3-6.2) | 2.1 (0.4-6.4) |
| intervention | | | | |
| 1 week | 1.6 (0.3-22.4) | 1.2 (0.1-3.4) | 0.7 (0.3-7.9) | 1.5 (0.2-14.0) |
| 2 weeks | 1.9 (0.3-7.5) | 1.4 (0.1-9.4) | 1.3 (0.3-5.6) | 1.7 (0.2-4.5) |
| 4 weeks | 2.0 (0.4-21.6) | 1.7 (0.1-8.1) | 1.3 (0.4-11.5) | 1.7 (0.3-4.8) |
| Systolic BP | | | | |
| (mmHg) | | | | |
| Baseline | 131.6 ± 10.7 | 132.6 ± 12.8 | 131.7 ± 14.1 | 131.0 ± 10.8 |
| run-in | | | | |
| Baseline | 130.4 ± 12.4 | 134.7 ± 13.9 | 133.5 ± 17.3 | 128.4 ± 9.2 |
| intervention | | | | |
| 1 week | 130.9 ± 11.8 | 130.7 ± 12.3 | 129.6 ± 12.9 | 127.2 ± 12.1 |
| 2 weeks | 132.0 ± 14.1 | 129.5 ± 12.6 | 132.0 ± 11.3 | 127.9 ± 10.1 |
| 4 weeks | 132.1 ± 10.6 | 131.8 ± 12.5 | 130.6 ± 13.5 | 127.6 ± 10.9 |
| Run-out | 130.7 ± 13.1 | 131.3 ± 13.2 | 130.1 ± 11.5 | 128.0 ± 10.1 |
| Diastolic BP | | | | |
| (mmHg) | | | | |
| Baseline | 82.8 ± 8.3 | 83.6 ± 9.9 | 84.0 ± 7.6 | 84.3 ± 6.3 |
| run-in | | | | |
| Baseline | 82.8 ± 6.7 | 84.6 ± 9.6 | 83.8 ± 7.8 | 83.3 ± 7.4 |
| intervention | | | | |
| 1 week | 82.7 ± 5.8 | 82.5 ± 9.6 | 82.4 ± 8.0 | 82.9 ± 9.6 |
| 2 weeks | 84.4 ± 7.4 | 83.5 ± 8.2 | 83.7 ± 7.3 | 82.4 ± 8.1 |
| 4 weeks | 82.9 ± 6.0 | 82.7 ± 9.0 | 83.0 ± 8.7 | 83.0 ± 7.1 |
| Run-out | 83.6 ± 5.1 | 82.3 ± 8.7 | 82.7 ± 8.6 | 83.1 ± 8.0 |
| Resting | | | | |
| heart rate | | | | |
| (beats per | | | | |
| minute) | | | | |
| Baseline | 66 ± 12 | 60 ± 7 | 59 ± 8 | 63 ± 8 |
| 4 weeks | 65 ± 11 | 61 ± 8 | 60 ± 7 | 61 ± 8 |

Values are means ± SD

### Example 9—Clinical Trial with Human Subjects—Increased Daily Dose of R. rubrum Cells

To establish further the observed dose-response effect of daily administration of 0.25 gram dried R. rubrum cells daily, 0.5 gram daily and 1.0 gram daily, when the lowering of LDL-cholesterol concentration in the blood of the treated human subjects is considered and when the essentially unaltered values for the series of blood parameters and SBP and DBP are considered, a double blind randomized controlled clinical trial is conducted similar to the clinical trial outlined in Example 8, with the difference that now groups of healthy human subjects receive a daily dose of 1.0, 2.0, 3.0, 4.0 or 5.0 grams R. rubrum cells. The design of this study is outlined in FIG. 10.

1. A method of lowering LDL-cholesterol concentration in blood of a human subject, the method comprising orally administering to the human subject a composition comprising of or consisting of Rhodospirillum rubrum cells.

2. The method of claim 1, wherein the concentration of at least one of ALT, AST, γGT, creatinine, NT-ProBNP, vWF, c-Troponin T, plasma glucose and hsCRP remains within a standard normal range and/or remains essentially constant in the serum and/or plasma of the human subject upon administration of the Rhodospirillum rubrum cells to said human subject, preferably all the concentrations remain essentially constant and/or remain within the standard normal range, preferably compared to the concentration(s) determined for said at least one of ALT, AST, γGT, creatinine, NT-ProBNP, vWF, c-Troponin T, plasma glucose and hsCRP before the start of the first administration of the Rhodospirillum rubrum cells and/or during the subsequent further administration(s) of the Rhodospirillum rubrum cells.

3. The method of claim 1, wherein diastolic blood pressure and/or systolic blood pressure of the human subject remain(s) within a standard normal range and/or remain(s) essentially constant upon administration of the Rhodospirillum rubrum cells to said human subject, preferably compared to said blood pressure(s) determined before the start of the first administration of the Rhodospirillum rubrum cells and/or during the subsequent further administration(s) of the Rhodospirillum rubrum cells.

4. The method of claim 1, wherein the HDL-cholesterol concentration in the blood of the human subject remains unaltered or increases or is increased during the course of the administration of the Rhodospirillum rubrum cells, preferably once or twice daily administration, preferably compared to the HDL-cholesterol concentration determined before the start of the first administration of the Rhodospirillum rubrum cells and/or during the subsequent further administration(s) of the Rhodospirillum rubrum cells.

5. The method of claim 1, wherein the human subject has a total plasma cholesterol level of 1.5 mM-16.0 mM, preferably 2.0 mM-12.0 mM, more preferably 3.0 mM-10.0 mM, most preferably 5.0 mM-8.0 mM, before and/or at the start of the first administration and/or during the subsequent further administration(s) of the Rhodospirillum rubrum cells to said human subject.

6. The method of claim 1, wherein the Rhodospirillum rubrum cells are administered orally to the human subject.

7. The method of claim 1, wherein the Rhodospirillum rubrum cells are administered to a healthy human subject, preferably a healthy human subject with a total cholesterol concentration in the blood before the first administration of Rhodospirillum rubrum cells and/or during the subsequent further administration(s) of the Rhodospirillum rubrum cells to said human subject, of at least 2.0 mM, such as 3.0 mM-14.0 mM or 5.0 mM-8.0 mM.

8. The method of claim 1, wherein administering the Rhodospirillum rubrum cells to the human subject results in a decrease of the LDL-cholesterol concentration in the plasma of said human subject, preferably with at least 1%, preferably at least 3%, more preferably at least 5%, more preferably at least 8%, most preferably at least 20%, based on the plasma LDL-cholesterol concentration prior to the first administration of the Rhodospirillum rubrum cells to said human subject, and wherein optionally the plasma HDL-cholesterol concentration remains essentially unaltered or decreases to a smaller extent than the decrease in the plasma LDL-cholesterol concentration, or wherein the plasma HDL-cholesterol concentration increases, preferably the plasma HDL-cholesterol concentration remains essentially unaltered or increases, based on the plasma HDL-cholesterol concentration prior to the first administration of the Rhodospirillum rubrum cells to said human subject.

9. The method of claim 1, wherein the Rhodospirillum rubrum cells are formulated as a granulate of dried Rhodospirillum rubrum cells obtained by subjecting the cells, preferably freshly cultured cells, to drying, preferably refractive drying at 55° C.-100° C., preferably at 60° C.-100° C., more preferably at 60° C.-70° C., preferably a granulate provided in a capsule such as a gelatin capsule or provided in a sachet.

10. The method of claim 1, wherein a daily dose of Rhodospirillum rubrum cells to be administered to a human subject is at least 1.0 g, such as at least 3.0 g, preferably 1.0-100 g, more preferably 2.0-50 g, most preferably 4.0-20 g, such as 5.0 gram.

11. A pharmaceutical composition comprising a pharmaceutically effective amount of Rhodospirillum rubrum cells and optionally a pharmaceutically acceptable excipient, for the lowering of LDL-cholesterol in blood plasma of a human subject.

12. The pharmaceutical composition of claim 11, wherein the concentration of at least one of ALT, AST, γGT, creatinine, NT-ProBNP, vWF, c-Troponin T, plasma glucose and hsCRP remains within a standard normal range and/or remains essentially constant in the serum and/or plasma of the human subject upon administration of the pharmaceutical composition to said human subject, preferably all the concentrations remain essentially constant and/or remain within the standard normal range, preferably compared to the concentration(s) determined for said at least one of ALT, AST, γGT, creatinine, NT-ProBNP, vWF, c-Troponin T, plasma glucose and hsCRP before the start of the first administration of the pharmaceutical composition and/or during the subsequent one or more further administrations of the pharmaceutical composition.

13. The pharmaceutical composition of claim 11, wherein diastolic blood pressure and/or systolic blood pressure of the human subject remain(s) within a standard normal range and/or remain(s) essentially constant upon administration of the pharmaceutical composition to said human subject, preferably compared to said blood pressure(s) determined before the start of the first administration of the pharmaceutical composition and/or during the subsequent further administration(s) of the pharmaceutical composition.

14. The pharmaceutical composition of claim 11, wherein the human subject has a total plasma cholesterol level of 1.5 mM-16.0 mM, preferably 2.0 mM-12.0 mM, more preferably 3.0 mM-10.0 mM, most preferably 5.0 mM-8.0 mM, before and/or at the start of the first administration and/or during the subsequent administration(s) of the pharmaceutical composition to said human subject.

15. The pharmaceutical composition of claim 11, wherein the Rhodospirillum rubrum cells are the sole active pharmaceutical ingredient in said pharmaceutical composition.

16. The pharmaceutical composition of claim 11, wherein the Rhodospirillum rubrum cells are administered to the human subject as replacement therapy such as therapy replacing a statin and/or ezetimibe, or are administered to the human subject in combination with a lower dose of (a) LDL-cholesterol lowering pharmaceutical compound(s) administered to the human subject than the dose of such (a) LDL-cholesterol lowering pharmaceutical compound(s) administered to the human subject before administration of the pharmaceutical composition started.

17. The pharmaceutical composition of claim 11, wherein a daily dose of the pharmaceutical composition contains at least 1.0 g Rhodospirillum rubrum cells, such as at least 2.0 g or 3.0 g, preferably 1.0-100 g, more preferably 2.0-50 g, most preferably 4.0-20 g such as 5.0-10.0 gram.

18. A food supplement with cholesterol-lowering properties when orally ingested by a human subject, comprising Rhodospirillum rubrum cells, wherein the Rhodospirillum rubrum cells are dried Rhodospirillum rubrum cell granules obtained by subjecting Rhodospirillum rubrum cells, preferably freshly cultured cells, to drying, preferably refractive drying at 55° C.-100° C., preferably at 60° C.-100° C., preferably Rhodospirillum rubrum cell granules provided in a capsule such as a gelatin capsule or in a sachet.

19. A foodstuff comprising a food supplement of claim 18.
